(19) United States
(12) Patent Application Publication
(10) Pub. No.: US 2010/0099651 A1
(43) Pub. Date: Apr. 22, 2010

(54) PHARMACEUTICAL COMPOSITIONS FOR TREATMENT OF RESPIRATORY AND GASTROINTESTINAL DISORDERS

(75) Inventors: Birgit Jung, Laupheim (DE); Frank Himmelsbach, Mittelbiberach (DE); Gerald Pohl, Biberach (DE)

Correspondence Address: MICHAEL P. MORRIS;BOEHRINGER INGELHEIM USA CORPORATION, 900 RIDGEBURY ROAD, RIDGEFIELD, CT, 06877-0368, US

(73) Assignee: BOEHRINGER INGELHEIM INTERNATIONAL GMBH, Ingelheim (DE)

(21) Appl. No.: 12/446,794

(22) PCT Filed: Oct. 23, 2007
(86) PCT No.: PCT/EP07/61355

§ 371 Date: Jun. 18, 2009

Related U.S. Application Data
(60) Provisional application No. 60/862,990, filed on Oct. 26, 2006.

(51) Int. Cl. A61K 31/517; A61K 31/5377; A61K 31/56; A61P 11/00

(52) U.S. Cl. 514/171; 514/234.5; 514/266.22

(57) ABSTRACT

The present invention relates to novel pharmaceutical compositions comprising at least one EGFR kinase inhibitor and at least one additional active compound selected from beta-2 mimetics, steroids, PDE-IV inhibitors, p38 MAP kinase inhibitors, NK₁ antagonists, anticholinergics and endothelin antagonists, processes for preparing the compositions and the use thereof as medicament in the treatment of respiratory or gastrointestinal complaints, as well as inflammatory diseases of the joints, the skin or the eyes.

The present invention relates to novel pharmaceutical compositions comprising one or more, preferably one, selected EGFR kinase inhibitors 1, and at least one additional active compound 2 processes for preparing them and their use as medicament in the treatment of respiratory or gastrointestinal complaints, as well as inflammatory diseases of the joints, the skin or the eyes.

### DETAILED DESCRIPTION OF THE INVENTION

In a first aspect the present invention relates to pharmaceutical compositions comprising at least one EGFR kinase inhibitor 1 selected from the group consisting of
(1.1) 4-[(3-chloro-4-fluoro-phenyl)amino]-6-[2-((S)-6-methyl-2-oxo-morpholin-4-yl)-ethoxy]-7-methoxy-quinazoline,
(1.2) 4-[(3-chloro-4-fluoro-phenyl)amino]-6-[2-(2,2-dimethyl-6-oxo-morpholin-4-yl)-ethoxy]-7-methoxy-quinazoline,
(1.3) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(1.4) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(1.5) 4-[(3-chloro-4-fluoro-phenyl)amino]-6-[1-(tert.-butyloxycarbonyl)-piperidin-4-yloxy]-7-methoxy-quinazoline,
(1.6) 4-[(3-chloro-4-fluoro-phenyl)amino]-6-(trans-4-amino-cyclohexan-1-yloxy)-7-methoxy-quinazoline,
(1.7) 4-[(3-chloro-4-fluoro-phenyl)amino]-6-(trans-4-methansulfonylamino-cyclohexan-1-yloxy)-7-methoxy-quinazoline,
(1.8) 4-[(3-chloro-4-fluoro-phenyl)amino]-6-(tetrahydropyran-3-yloxy)-7-methoxy-quinazoline,
(1.9) 4-[(3-chloro-4-fluoro-phenyl)amino]-6-(1-methyl-piperidin-4-yloxy)-7-methoxy-quinazoline,
(1.10) 4-[(3-chloro-4-fluoro-phenyl)amino]-6-{1-[(morpholin-4-yl)-carbonyl]-piperidin-4-yloxy}-7-methoxy-quinazoline,
(1.11) 4-[(3-chloro-4-fluoro-phenyl)amino]-6-{1-[methoxymethyl)carbonyl]-piperidin-4-yloxy}-7-methoxy-quinazoline,
(1.12) 4-[(3-chloro-4-fluoro-phenyl)amino]-6-(piperidin-3-yloxy)-7-methoxy-quinazoline,
(1.13) 4-[(3-chloro-4-fluoro-phenyl)amino]-6-[1-(2-acetylamino-ethyl)-piperidin-4-yl-oxy]-7-methoxy-quinazoline,
(1.14) 4-[(3-chloro-4-fluoro-phenyl)amino]-6-(tetrahydropyran-4-yloxy)-7-ethoxy-quinazoline
(1.15) 4-[(3-chloro-4-fluoro-phenyl)amino]-6-((S)-tetrahydrofuran-3-yloxy)-7-hydroxy-quinazoline,
(1.16) 4-[(3-chloro-4-fluoro-phenyl)amino]-6-(tetrahydropyran-4-yloxy)-7-(2-methoxy-ethoxy)-quinazoline,
(1.17) 4-[(3-chloro-4-fluoro-phenyl)amino]-6-{trans-4-[(dimethylamino)sulfonylamino]-cyclohexan-1-yloxy}-7-methoxy-quinazoline,
(1.18) 4-[(3-chloro-4-fluoro-phenyl)amino]-6-{trans-4-[(morpholin-4-yl)carbonylamino]-cyclohexan-1-yloxy}-7-methoxy-quinazoline,
(1.19) 4-[(3-chloro-4-fluoro-phenyl)amino]-6-{trans-4-[(morpholin-4-yl)sulfonylamino]-cyclohexan-1-yloxy}-7-methoxy-quinazoline,
(1.20) 4-[(3-chloro-4-fluoro-phenyl)amino]-6-(tetrahydropyran-4-yloxy)-7-(2-acetylamino-ethoxy)-quinazoline,
(1.21) 4-[(3-chloro-4-fluoro-phenyl)amino]-6-(tetrahydropyran-4-yloxy)-7-(2-methansulfonylamino-ethoxy)-quinazoline,
(1.22) 4-[(3-chloro-4-fluoro-phenyl)amino]-6-{1-[(piperidin-1-yl)carbonyl]-piperidin-4-yloxy}-7-methoxy-quinazoline,
(1.23) 4-[(3-chloro-4-fluoro-phenyl)amino]-6-(1-aminocarbonylmethyl-piperidin-4-yloxy)-7-methoxy-quinazoline,
(1.24) 4-[(3-chloro-4-fluoro-phenyl)amino]-6-(cis-4-{N-[tetrahydropyran-4-yl)carbonyl]-N-methyl-amino}-cyclohexan-1-yloxy)-7-methoxy-quinazoline,
(1.25) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.26) 4-[(3-chloro-4-fluoro-phenyl)amino]-6-(cis-4-{N-[(morpholin-4-yl)sulfonyl]-N-methyl-amino}-cyclohexan-1-yloxy)-7-methoxy-quinazoline,
(1.27) 4-[(3-chloro-4-fluoro-phenyl)amino]-6-(trans-4-ethansulfonylamino-cyclohexan-1-yloxy)-7-methoxy-quinazoline,
(1.28) 4-[(3-chloro-4-fluoro-phenyl)amino]-6-(1-methansulfonyl-piperidin-4-yloxy)-7-ethoxy-quinazoline,
(1.29) 4-[(3-chloro-4-fluoro-phenyl)amino]-6-(1-methansulfonyl-piperidin-4-yloxy)-7-(2-methoxy-ethoxy)-quinazoline,
(1.30) 4-[(3-chloro-4-fluoro-phenyl)amino]-6-[1-(2-methoxy-acetyl)-piperidin-4-yloxy]-7-(2-methoxy-ethoxy)-quinazoline,
(1.31) 4-[(3-chloro-4-fluoro-phenyl)amino]-6-(cis-4-acetylamino-cyclohexan-1-yloxy)-7-methoxy-quinazoline,
(1.32) 4-[(3-ethinyl-phenyl)amino]-6-[1-(tert.-butyloxycarbonyl)-piperidin-4-yloxy]-7-methoxy-quinazoline,
(1.33) 4-[(3-ethinyl-phenyl)amino]-6-(tetrahydropyran-4-yloxy]-7-methoxy-quinazoline,
(1.34) 4-[(3-chloro-4-fluoro-phenyl)amino]-6-(cis-4-{N-[piperidin-1-yl)carbonyl]-N-methyl-amino}-cyclohexan-1-yloxy)-7-methoxy-quinazoline,
(1.35) 4-[(3-chloro-4-fluoro-phenyl)amino]-6-(cis-4-{N-[(4-methyl-piperazin-1-yl)-carbonyl]-N-methyl-amino}-cyclohexan-1-yloxy)-7-methoxy-quinazoline,
(1.36) 4-[(3-chloro-4-fluoro-phenyl)amino]-6-{cis-4-[(morpholin-4-yl)carbonylamino]-cyclohexan-1-yloxy}-7-methoxy-quinazoline,
(1.37) 4-[(3-chloro-4-fluoro-phenyl)amino]-6-{1-[2-(2-oxopyrrolidin-1-yl)ethyl]-piperidin-4-yloxy}-7-methoxy-quinazoline,
(1.38) 4-[(3-chloro-4-fluoro-phenyl)amino]-6-{1-[(morpholin-4-yl)carbonyl]-piperidin-4-yloxy}-7-(2-methoxy-ethoxy)-quinazoline,
(1.39) 4-[(3-ethinyl-phenyl)amino]-6-(1-acetyl-piperidin-4-yloxy)-7-methoxy-quinazoline,
(1.40) 4-[(3-ethinyl-phenyl)amino]-6-(1-methyl-piperidin-4-yloxy)-7-methoxy-quinazoline,
(1.41) 4-[(3-ethinyl-phenyl)amino]-6-(1-methansulfonyl-piperidin-4-yloxy)-7-methoxy-quinazoline,
(1.42) 4-[(3-chloro-4-fluoro-phenyl)amino]-6-(1-methyl-piperidin-4-yloxy)-7(2-methoxy-ethoxy)-quinazoline,
(1.43) 4-[(3-chloro-4-fluoro-phenyl)amino]-6-(1-isopropyloxycarbonyl-piperidin-4-yloxy)-7-methoxy-quinazoline,
(1.44) 4-[(3-chloro-4-fluoro-phenyl)amino]-6-(cis-4-methylamino-cyclohexan-1-yloxy)-7-methoxy-quinazoline,
(1.45) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.46) 4-[(3-ethinyl-phenyl)amino]-6-(piperidin-4-yloxy)-7-methoxy-quinazoline,
(1.47) 4-[(3-ethinyl-phenyl)amino]-6-[1-(2-methoxy-acetyl)-piperidin-4-yloxy]-7-methoxy-quinazoline,
(1.48) 4-[(3-ethinyl-phenyl)amino]-6-{1-[(morpholin-4-yl)carbonyl]-piperidin-4-yloxy}-7-methoxy-quinazoline,
(1.49) 4-[(3-chloro-4-fluoro-phenyl)amino]-6-{1-[(cis-2,6-dimethyl-morpholin-4-yl)carbonyl]-piperidin-4-yloxy}-7-methoxy-quinazoline,
(1.50) 4-[(3-chloro-4-fluoro-phenyl)amino]-6-{1-[(2-methyl-morpholin-4-yl)carbonyl]-piperidin-4-yloxy}-7-methoxy-quinazoline,
(1.51) 4-[(3-chloro-4-fluoro-phenyl)amino]-6-{1-[(S,S)-(2-oxa-5-aza-bicyclo[2.2.1]-hept5-yl)carbonyl)-piperidin-4-yloxy}-7-methoxy-quinazoline,
(1.52) 41(3-chloro-4-fluoro-phenyl)amino]-6-{1-[(N-methyl-N-2-methoxyethyl-amino)carbonyl]-piperidin-4-yloxy}-7-methoxy-quinazoline,
(1.53) 4-[(3-chloro-4-fluoro-phenyl)amino]-6-(1-ethyl-piperidin-4-yloxy)-7-methoxy-quinazoline,
(1.54) 4-[(3-chloro-4-fluoro-phenyl)amino]-6-{1-[(2-methoxyethyl)carbonyl]-piperidin-4-yloxy}-7-methoxy-quinazoline,
(1.55) 4-[(3-chloro-4-fluoro-phenyl)amino]-6-{1-[(3-methoxypropyl-amino)-carbonyl]-piperidin-4-yloxy}-7-methoxy-quinazoline,
(1.56) 4-[(3-chloro-4-fluoro-phenyl)amino]-6-[cis-4-(N-methansulfonyl-N-methyl-amino)-cyclohexan-1-yloxy]-7-methoxy-quinazoline,
(1.57) 4-[(3-chloro-4-fluoro-phenyl)amino]-6-[cis-4-(N-acetyl-N-methyl-amino)-cyclohexan-1-yloxy]-7-methoxy-quinazoline,
(1.58) 4-[(3-chloro-4-fluoro-phenyl)amino]-6-(trans-4-methylamino-cyclohexan-1-yl-oxy)-7-methoxy-quinazoline,
(1.59) 4-[(3-chloro-4-fluoro-phenyl)amino]-6-[trans-4-(N-methansulfonyl-N-methyl-amino)-cyclohexan-1-yloxy]-7-methoxy-quinazoline,
(1.60) 4-[(3-chloro-4-fluoro-phenyl)amino]-6-(trans-4-dimethylamino-cyclohexan-1-yloxy)-7-methoxy-quinazoline,
(1.61) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(1.62) 4-[(3-chloro-4-fluoro-phenyl)amino]-6-{1-[2-(2-oxo-3-methyl-imidazolidin-1-yl)-ethyl]-piperidin-4-yloxy}-7-methoxy-quinazoline
(1.63) 4-[(3-chloro-4-fluoro-phenyl)amino]-6-{1-[2-(2-oxo-hexahydropyrimidin-1-yl)-ethyl]-piperidin-4-yloxy}-7-methoxy-quinazoline,
(1.64) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.65) 4-[(3-chloro-4-fluoro-phenyl)amino]-6-(1-methansulfonyl-piperidin-4-yloxy)-7-methoxy-quinazoline,
(1.66) 4-[(3-chloro-4-fluoro-phenyl)amino]-6-(1-cyano-piperidin-4-yloxy)-7-methoxy-quinazoline,
0.67) 4-[(3-chloro-4-fluoro-phenyl)amino]-8-(tetrahydropyran-4-yloxy)-7-methoxy-quinazoline,
(1.68) 4-[(3-chloro-4-fluoro-phenyl)amino]-6-(1-methylcarbonyl-piperidin-4-yloxy)-7-methoxy-quinazoline,
(1.69) 4-[(3-chloro-4-fluoro-phenyl)amino]-6-(1-dimethylaminoacetyl-piperidin-4-yloxy)-7-methoxy-quinazoline,
(1.70) 4-[(3-chloro-4-fluoro-phenyl)amino]-6-{1-[(dimethylamino)carbonylmethyl]-piperidin-4-yloxy}-7-methoxy-quinazoline,
(1.71) 4-[(3-chloro-4-fluoro-phenyl)amino]-6-(1-methansulfonyl-piperidin-4-yloxy)-quinazoline,
   optionally in the form of tautomers, racemates, enantiomers, diastereomers, pharmacologically acceptable acid addition salts, solvates or hydrates thereof,
   and further comprising one or more additional active compounds 2 selected from the classes consisting of beta-2 mimetics 2a, steroids 2b, PDE-IV inhibitors 2c, p38 MAP kinase inhibitors 2d, NK₁ antagonists 2e, anticholinergics 2f and endothelin antagonists 2g, optionally together with one or more pharmaceutically acceptable excipients or carriers.

In the pharmaceutical compositions according to the present invention the EGFR kinase inhibitors 1 may be contained in a form selected from tautomers, optical isomers, enantiomers, racemates, diastereomers, pharmacologically acceptable acid addition salts, solvates or hydrates, as far as such forms exist, depending on the individual compound. Pharmaceutical compositions comprising one or more, preferably one, compound 1 in form of a substantially pure enantiomer are preferred.

Pharmacological acceptable acid addition salts of EGFR kinase inhibitors 1 comprise salts selected from the group consisting of the hydrochloride, hydrobromide, hydroiodide, hydrosulphite, hydrophosphate, hydromethanesulphonate, hydronitrate, hydromaleate, hydroacetate, hydrobenzoate, hydrocitrate, hydrofumarate, hydrotartrate, hydrolactate, hydrooxalate, hydrosuccinate, hydrobenzoate and hydro-p-toluolsulphonate, preferably hydrochloride, hydrobromide, hydrosulphite, hydrophosphate, hydromaleate, hydrofumarate and hydromethansulphonate. Some of the compounds may add more than one equivalent acid, e.g. two equivalents. The salts of hydrochloric acid, methanesulphonic acid, maleic acid, benzoic acid and acetic acid are especially preferred.

In a first preferred embodiment of the invention the pharmaceutical composition is a binary combination, containing an EGFR kinase inhibitor 1 and an active compound 2 selected from one of the classes 2a, 2b, 2c, 2d, 2e, 2f and 2g. optionally together with one or more pharmaceutically acceptable excipients or carriers.

In another preferred embodiment of the invention the pharmaceutical composition is a binary combination, wherein the active compound 2 is a beta-2 mimetic 2a.

In another preferred embodiment of the invention the pharmaceutical composition is a binary combination, wherein the active compound 2 is a steroid 2b.

In another preferred embodiment of the invention the pharmaceutical composition is a binary combination, wherein the active compound 2 is a PDE-IV inhibitor 2c.

In another preferred embodiment of the invention the pharmaceutical composition is a binary combination, wherein the active compound 2 is a p38 MAP kinase inhibitor 2d.

In another preferred embodiment of the invention the pharmaceutical composition is a binary combination, wherein the active compound 2 is a NK₁ antagonists 2e.

In another preferred embodiment of the invention the pharmaceutical composition is a binary combination, wherein the active compound 2 is an anticholinergic 2f. In another preferred embodiment of the invention the pharmaceutical composition is a binary combination, wherein the active compound 2 is an endothelin antagonist 2g.

The pharmaceutical compositions according to the invention comprising at least one EGFR kinase inhibitor 1 and at least one additional active compound 2 are not restricted to binary combinations of actives. The combinations disclosed exemplary below comprising an EGFR kinase inhibitor 1 together with an additional active compound 2 may comprise a third or a third and a fourth, preferably a third active compound, also selected from the group consisting of beta-2 mimetics 2a, steroids 2b, PDE-IV inhibitors 2c, p38 MAP kinase inhibitors 2d, NK₁ antagonists 2e and anticholinergic 2f and endothelin antagonist 2g. All components 2a to 2g. mentioned specifically hereinafter are described in the prior art.

In another preferred embodiment of the invention the pharmaceutical composition is a ternary combination, containing an EGFR kinase inhibitor 1 and two active compound selected from the class of beta-2 mimetics 2a and an active compound selected from the class of steroids 2b, optionally together with one or more pharmaceutically acceptable excipients or carriers.

In another preferred embodiment of the invention the pharmaceutical composition is a ternary combination, containing two EGFR kinase inhibitors 1 and an active compound selected from one of the classes 2a, 2b, 2c, 2d, 2e, 2f and 2g, optionally together with one or more pharmaceutically acceptable excipients or carriers.

In another preferred embodiment of the invention the pharmaceutical composition is a quarternary combination, containing two EGFR kinase inhibitors 1 and two active compounds selected from either one or from two different classes of 2a, 2b, 2c, 2d, 2e, 2f and 2g optionally together with one or more pharmaceutically acceptable excipients or carriers.

In another preferred embodiment of the invention the pharmaceutical composition is a quarternary combination, containing two EGFR kinase inhibitors 1 and two active compounds selected from either one or from two different classes of 2b, 2d and 2e, optionally together with one or more pharmaceutically acceptable excipients or carriers.

Any reference to an EGFR kinase inhibitor 1 within the scope of the present invention should be understood as a reference to any specific EGFR kinase inhibitor selected from compounds 1.1 to 1.71. mentioned hereinbefore. Analogously, any reference to to an active compound selected from the classes 2a, 2b, 2c, 2d, 2e, 2f and 2g within the scope of the present invention should be understood as a reference to any active compound of these classes mentioned specifically hereinbelow.

One embodiment of the invention is a pharmaceutical composition comprising an EGFR kinase inhibitor 1 and a beta-2 mimetic 2a. Binary compositions containing only one active 1 and one active 2a, optionally together with one or more pharmaceutically acceptable excipients or carriers, are preferred.

In the pharmaceutical combinations according to the invention beta-2-mimetic 2a is selected from the group consisting of the compounds of formula 2a.I

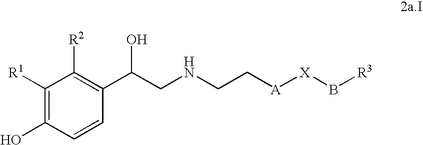

wherein
A denotes phenylen or —C₁-C₅-alkylen;
B denotes a group selected from a single bond, phenylen, —C₁-C₅-alkylen and
   —C₁-C₃-alkylen-O—C₁-C₃-alkylen which is optionally substituted by OH or —O—C₁-C₄-alkyl;
X denotes —NH— or —O—;
R¹ denotes —CH₂—OH, or —NH—CHO;
R² denotes hydrogen, or
R¹ and R² together —NH—CO—CH═CH—
R³ denotes phenyl which is optionally substituted by one or two groups selected from among —C₁-C₄-alkyl, halogen, —O—C₁-C₄-alkyl, alkylene-NH₂, —SO₂NH₂, —NH—CO—NH₂, —SO₂—C₁-C₅-alkyl and —SO₂—C₃-C₆-cycloalkyl,
   optionally in the form of the racemates, the enantiomers, the diastereomers and optionally the pharmacologically acceptable acid addition salts and the hydrates thereof.

In the pharmaceutical combinations according to the invention preferred beta-2 agonists 2a.I are selected from the group consisting of
2-Hydroxy-5-(1-hydroxy-2-{2-[4-(2-hydroxy-2-phenyl-ethylamino)-phenyl]-ethylamino}-ethyl)-benzaldehyde 2a.I.1, N-[2-Hydroxy-5-(1-hydroxy-2-{2-[4-(2-hydroxy-2-phenyl-ethylamino)-phenyl]-ethylamino}-ethyl)-phenyl]-formamide 2a.I.2, 8-Hydroxy-5-(1-hydroxy-2-{2-[4-(6-methoxy-biphenyl-3-ylamino)-phenyl]-ethylamino}-ethyl)-1H-quinolin-2-one 2a.I.3,
8-Hydroxy-5-[1-hydroxy-2-(6-phenethylamino-hexylamino)-ethyl]-1H-quinolin-2-one 2a.I.4,
5-[2-(2-{4-[4-(2-Amino-2-methyl-propoxy)-phenylamino]-phenyl}-ethylamino)-1-hydroxy-ethyl]-8-hydroxy-1H-quinolin-2-one 2a.I.5,
[3-(4-{6-[2-Hydroxy-2-(4-hydroxy-3-hydroxymethyl-phenyl)-ethylamino]-hexyloxy}-butyl)-5-methyl-phenyl]-urea 2a.I.6,
4-(2-{6-[2-(2,6-Dichloro-benzyloxy)-ethoxy]-hexylamino}-1-hydroxy-ethyl)-2-hydroxymethyl-phenol 2a.I.7,
3-(3-{7-[2-Hydroxy-2-(4-hydroxy-3-hydroxymethyl-phenyl)-ethylamino]-heptyloxy}-propyl)-benzenesulfonamide 2a.I.8,
4-(2-{6-[4-(3-Cyclopentanesulfonyl-phenyl)-butoxy]-hexylamino}-1-hydroxy-ethyl)-2-hydroxymethyl-phenol 2a.I.9,
   optionally in the form of the racemates, the enantiomers, the diastereomers and optionally the pharmacologically acceptable acid addition salts and the hydrates thereof,
   or
   the beta-2 mimetic 2a is selected from the group consisting of
   N-Adamantan-2-yl-2-(3-{2-[2-hydroxy-2-(4-hydroxy-3-hydroxymethyl-phenyl)-ethylamino]-propyl}-phenyl)-acetamide 2a.1, 6-Hydroxy-8-{1-hydroxy-2-[2-(4-methoxy-phenyl)-1,1-dimethyl-ethylamino]-ethyl}-4H-benzo[1,4]oxazin-3-on 2a.2,6-Hydroxy-8-{1-hydroxy-2-[2-(4-phenoxy-essigsäureethylester)-1,1-dimethyl-ethylamino]-ethyl}-4H-benzo[1,4]oxazin-3-on 2a.3,6-Hydroxy-8-{1-hydroxy-2-[2-(4-phenoxy-essigsäure)-1,1-dimethyl-ethylamino]-ethyl}-4H-benzo[1,4]oxazin-3-on 2a.4, 8-{2-[1,1-Dimethyl-2-(2,4,6-trimethylphenyl)-ethylamino]-1-hydroxy-ethyl}-6-hydroxy-4H-benzo[1,4]oxazin-3-on 2a.5,6-Hydroxy-8-{1-hydroxy-2-[2-(4-hydroxy-phenyl)-1,1-dimethyl-ethylamino]-ethyl}-4H-benzo[1,4]oxazin-3-on 2a.6,6-Hydroxy-8-{1-hydroxy-2-[2-(4-isopropyl-phenyl)-1,1dimethyl-ethylamino]-ethyl}-4H-benzo[1,4]oxazin-3-on 2a.7, 8-{2-[2-(4-Ethyl-phenyl)-1,1-dimethyl-ethylamino]-1-hydroxy-ethyl}-6-hydroxy-4H-benzo[1,4]oxazin-3-on 2a.8, 8-{2-[2-(4-Ethoxy-phenyl)-1,1-dimethyl-ethylamino]-1-hydroxy-ethyl}-6-hydroxy-4H-benzo[1,4]oxazin-3-on 2a.9,4-(4-{2-[2-Hydroxy-2-(6-hydroxy-3-oxo-3,4-dihydro-2H-benzo[1,4]oxazin-8-yl)-ethylamino]-2-methyl-propyl}-phenoxy)-buttersäure 2a.10, 8-{2-[2-(3,4-Difluor-phenyl)-1,1-dimethyl-ethylamino]-1-hydroxy-ethyl}-6-hydroxy-4H-benzo[1,4]oxazin-3-on 2a.11, 2-Hydroxymethyl-4-{1-hydroxy-2-[6-(4-m-tolyl-butoxy)-hexylamino]-ethyl}-phenol 2a.12, 2-Hydroxymethyl-4-{1-hydroxy-2-[7-(3-m-tolyl-propoxy)-heptylamino]-ethyl}-phenol 2a.13 optionally in the form of the racemates, the enantiomers, the diastereomers and optionally the pharmacologically acceptable acid addition salts thereof, and the hydrates thereof.

Of the beta-2 mimetics mentioned above the compounds 2a.I.1, 2a.I.2, 2a.I.3, 2a.I.4, 2a.I.5, 2a.I.6, 2a.I.7, 2a.I.8, 2a.I.9, 2a.1, 2a.2, 2a.3, 2a.4, 2a.5, 2a.6, 2a.7, 2a.8, 2a.9, 2a.10, 2a.11, 2a.12 and 2a.13 are preferred, optionally in the form of the racemates, the enantiomers, the diastereomers and optionally the pharmacologically acceptable acid addition salts thereof, and the hydrates thereof.

Examples of pharmacologically acceptable acid addition salts of the betamimetics 2a according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2,4-difluorophenyl)salicylic acid or maleic acid. If desired, mixtures of the abovementioned acids may also be used to prepare the salts of 2a.

Any reference to the term betamimetics 2a also includes a reference to the relevant enantiomers or mixtures thereof.

In the pharmaceutical compositions according to the invention, the compounds 2a may be present in the form of their racemates, enantiomers or mixtures thereof. The separation of the enantiomers from the racemates may be carried out using methods known in the art (e.g. by chromatography on chiral phases, etc.).

The compounds 2a may also be present according to the invention in the form of the hydrates or solvates thereof.

Within the scope of the present invention the betamimetics 2a may possibly also be referred to as sympathomimetics or beta-2-agonists (β₂-agonists). All these terms are to be regarded as interchangeable for the purposes of the present invention.

Besides therapeutically effective quantities of 1 and 2a the pharmaceutical compositions may contain in addition a pharmaceutically acceptable carrier. The present invention encompasses both pharmaceutical compositions with or without pharmaceutically acceptable carriers.

Especially preferred pharmaceutical compositions according to the invention comprise the following specific combinations of EGFR kinase inhibitors 1 and beta-2 mimetics 2a, either as free bases or pharmacologically acceptable acid addition salts: 1.1 and 2a.I.1, 1.1 and 2a.I.2, 1.1 and 2a.I.3, 1.1 and 2a.I.4, 1.1 and 2a.I.5, 1.1 and 2a.I.6, 1.1 and 2a.I.7, 1.1 and 2a.I.8, 1.1 and 2a.I.9, 1.1 and 2a.1, 1.1 and 2a.2, 1.1 and 2a.3, 1.1 and 2a.4, 1.1 and 2a.5, 1.1 and 2a.6, 1.1 and 2a.7, 1.1 and 2a.8, 1.1 and 2a.9, 1.1 and 2a.10, 1.1 and 2a.11, 1.1 and 2a.12, 1.1 and 2a.13,

1.2 and 2a.I.1, 1.2 and 2a.I.2, 1.2 and 2a.I.3, 1.2 and 2a.I.4, 1.2 and 2a.I.5, 1.2 and 2a.I.6, 1.2 and 2a.I.7, 1.2 and 2a.I.8, 1.2 and 2a.I.9, 1.2 and 2a.1, 1.2 and 2a.2, 1.2 and 2a.3, 1.2 and 2a.4, 1.2 and 2a.5, 1.2 and 2a.6, 1.2 and 2a.7, 1.2 and 2a.8, 1.2 and 2a.9, 1.2 and 2a.10, 1.2 and 2a.11, 1.2 and 2a.12, 1.2 and 2a.13,
1.8 and 2a.I.1, 1.8 and 2a.I.2, 1.8 and 2a.I.3, 1.8 and 2a.I.4, 1.8 and 2a.I.5, 1.8 and 2a.I.6, 1.8 and 2a.I.7, 1.8 and 2a.I.8, 1.8 and 2a.I.9, 1.8 and 2a.1, 1.8 and 2a.2, 1.8 and 2a.3, 1.8 and 2a.4, 1.8 and 2a.5, 1.8 and 2a.6, 1.8 and 2a.7, 1.8 and 2a.8, 1.8 and 2a.9, 1.8 and 2a.10, 1.8 and 2a.11, 1.8 and 2a.12, 1.8 and 2a.13,
1.12 and 2a.I.1, 1.12 and 2a.I.2, 1.12 and 2a.I.3, 1.12 and 2a.I.4, 1.12 and 2a.I.5, 1.12 and 2a.I.6, 1.12 and 2a.I.7, 1.12 and 2a.I.8, 1.12 and 2a.I.9, 1.12 and 2a.1, 1.12 and 2a.2, 1.12 and 2a.3, 1.12 and 2a.4, 1.12 and 2a.5, 1.12 and 2a.6, 1.12 and 2a.7, 1.12 and 2a.8, 1.12 and 2a.9, 1.12 and 2a.10, 1.12 and 2a.11, 1.12 and 2a.12, 1.12 and 2a.13,
1.13 and 2a.I.1, 1.13 and 2a.I.2, 1.13 and 2a.I.3, 1.13 and 2a.I.4, 1.13 and 2a.I.5, 1.13 and 2a.I.6, 1.13 and 2a.I.7, 1.13 and 2a.I.8, 1.13 and 2a.I.9, 1.13 and 2a.1, 1.13 and 2a.2, 1.13 and 2a.3, 1.13 and 2a.4, 1.13 and 2a.5, 1.13 and 2a.6, 1.13 and 2a.7, 1.13 and 2a.8, 1.13 and 2a.9, 1.13 and 2a.10, 1.13 and 2a.11, 1.13 and 2a.12, 1.13 and 2a.13,
1.20 and 2a.I.1, 1.20 and 2a.I.2, 1.20 and 2a.I.3, 1.20 and 2a.I.4, 1.20 and 2a.I.5, 1.20 and 2a.I.6, 1.20 and 2a.I.7, 1.20 and 2a.I.8, 1.20 and 2a.I.9, 1.20 and 2a.1, 1.20 and 2a.2, 1.20 and 2a.3, 1.20 and 2a.4, 1.20 and 2a.5, 1.20 and 2a.6, 1.20 and 2a.7, 1.20 and 2a.8, 1.20 and 2a.9, 1.20 and 2a.10, 1.20 and 2a.11, 1.20 and 2a.12, 1.20 and 2a.13,
1.26 and 2a.I.1, 1.26 and 2a.I.2, 1.26 and 2a.I.3, 1.26 and 2a.I.4, 1.26 and 2a.I.5, 1.26 and 2a.I.6, 1.26 and 2a.I.7, 1.26 and 2a.I.8, 1.26 and 2a.I.9, 1.26 and 2a.1, 1.26 and 2a.2, 1.26 and 2a.3, 1.26 and 2a.4, 1.26 and 2a.5, 1.26 and 2a.6, 1.26 and 2a.7, 1.26 and 2a.8, 1.26 and 2a.9, 1.26 and 2a.10, 1.26 and 2a.11, 1.26 and 2a.12, 1.26 and 2a.13,
1.27 and 2a.I.1, 1.27 and 2a.I.2, 1.27 and 2a.I.3, 1.27 and 2a.I.4, 1.27 and 2a.I.5, 1.27 and 2a.I.6, 1.27 and 2a.I.7, 1.27 and 2a.I.8, 1.27 and 2a.I.9, 1.27 and 2a.1, 1.27 and 2a.2, 1.27 and 2a.3, 1.27 and 2a.4, 1.27 and 2a.5, 1.27 and 2a.6, and 2a.7, 1.27 and 2a.8, 1.27 and 2a.9, 1.27 and 2a.10, 1.27 and 2a.11, 1.27 and 2a.12, 1.27 and 2a.13, 1.27 1.28 and 2a.I.1, 1.28 and 2a.I.2, 1.28 and 2a.I.3, 1.28 and 2a.I.4, 1.28 and 2a.I.5, 1.28 and 2a.I.6, 1.28 and 2a.I.7, 1.28 and 2a.I.8, 1.28 and 2a.I.9, 1.28 and 2a.1, 1.28 and 2a.2, 1.28 and 2a.3, 1.28 and 2a.4, 1.28 and 2a.5, 1.28 and 2a.6, 1.28 and 2a.7, 1.28 and 2a.8, 1.28 and 2a.9, 1.28 and 2a.10, 1.28 and 2a.11, 1.28 and 2a.12, 1.28 and 2a.13,
1.33 and 2a.I.1, 1.33 and 2a.I.2, 1.33 and 2a.I.3, 1.33 and 2a.I.4, 1.33 and 2a.I.5, 1.33 and 2a.I.6, 1.33 and 2a.I.7, 1.33 and 2a.I.8, 1.33 and 2a.I.9, 1.33 and 2a.1, 1.33 and 2a.2, 1.33 and 2a.3, 1.33 and 2a.4, 1.33 and 2a.5, 1.33 and 2a.6, 1.33 and 2a.7, 1.33 and 2a.8, 1.33 and 2a.9, 1.33 and 2a.10, 1.33 and 2a.11, 1.33 and 2a.12, 1.33 and 2a.13,
1.45 and 2a.I.1, 1.45 and 2a.I.2, 1.45 and 2a.I.3, 1.45 and 2a.I.4, 1.45 and 2a.I.5, 1.45 and 2a.I.6, 1.45 and 2a.I.7, 1.45 and 2a.1.8, 1.45 and 2a.I.9, 1.45 and 2a.1, 1.45 and 2a.2, 1.45 and 2a.3, 1.45 and 2a.4, 1.45 and 2a.5, 1.45 and 2a.6, 1.45 and 2a.7, 1.45 and 2a.8, 1.45 and 2a.9, 1.45 and 2a.10, 1.45 and 2a.11, 1.45 and 2a.12, 1.45 and 2a.13,
1.46 and 2a.I.1, 1.46 and 2a.I.2, 1.46 and 2a.I.3, 1.46 and 2a.I.4, 1.46 and 2a.I.5, 1.46 and 2a.I.6, 1.46 and 2a.I.7, 1.46 and 2a.I.8, 1.46 and 2a.I.9, 1.46 and 2a.1, 1.46 and 2a.2, 1.46 and 2a.3, 1.46 and 2a.4, 1.46 and 2a.5, 1.46 and 2a.6, 1.46 and 2a.7, 1.46 and 2a.8, 1.46 and 2a.9, 1.46 and 2a.10, 1.46 and 2a.11, 1.46 and 2a.12, 1.46 and 2a.13,
1.47 and 2a.I.1, 1.47 and 2a.I.2, 1.47 and 2a.I.3, 1.47 and 2a.I.4, 1.47 and 2a.I.5, 1.47 and 2a.I.6, 1.47 and 2a.I.7, 1.47 and 2a.I.8, 1.47 and 2a.I.9, 1.47 and 2a.1, 1.47 and 2a.2, 1.47 and 2a.3, 1.47 and 2a.4, 1.47 and 2a.5, 1.47 and 2a.6, 1.47 and 2a.7, 1.47 and 2a.8, 1.47 and 2a.9, 1.47 and 2a.10, 1.47 and 2a.11, 1.47 and 2a.12, 1.47 and 2a.13,
1.48 and 2a.I.1, 1.48 and 2a.1.2, 1.48 and 2a.I.3, 1.48 and 2a.I.4, 1.48 and 2a.I.5, 1.48 and 2a.I.6, 1.48 and 2a.I.7, 1.48 and 2a.I.8, 1.48 and 2a.I.9, 1.48 and 2a.1, 1.48 and 2a.2, 1.48 and 2a.3, 1.48 and 2a.4, 1.48 and 2a.5, 1.48 and 2a.6, 1.48 and 2a.7, 1.48 and 2a.8, 1.48 and 2a.9, 1.48 and 2a.10, 1.48 and 2a.11, 1.48 and 2a.12, 1.48 and 2a.13,
1.62 and 2a.I.1, 1.62 and 2a.1.2, 1.62 and 2a.I.3, 1.62 and 2a.I.4, 1.62 and 2a.I.5, 1.62 and 2a.I.6, 1.62 and 2a.I.7, 1.62 and 2a.1.8, 1.62 and 2a.I.9, 1.62 and 2a.1, 1.62 and 2a.2, 1.62 and 2a.3, 1.62 and 2a.4, 1.62 and 2a.5, 1.62 and 2a.6, 1.62 and 2a.7, 1.62 and 2a.8, 1.62 and 2a.9, 1.62 and 2a.10, 1.62 and 2a.11, 1.62 and 2a.12, 1.62 and 2a.13,
1.64 and 2a.I.1, 1.64 and 2a.I.2, 1.64 and 2a.I.3, 1.64 and 2a.I.4, 1.64 and 2a.I.5, 1.64 and 2a.I.6, 1.64 and 2a.I.7, 1.64 and 2a.I.8, 1.64 and 2a.I.9, 1.64 and 2a.1, 1.64 and 2a.2, 1.64 and 2a.3, 1.64 and 2a.4, 1.64 and 2a.5, 1.64 and 2a.6, 1.64 and 2a.7, 1.64 and 2a.8, 1.64 and 2a.9, 1.64 and 2a.10, 1.64 and 2a.11, 1.64 and 2a.12, 1.64 and 2a.13,
1.67 and 2a.I.1, 1.67 and 2a.I.2, 1.67 and 2a.1.3, 1.67 and 2a.I.4, 1.67 and 2a.I.5, 1.67 and 2a.I.6, 1.67 and 2a.I.7, 1.67 and 2a.I.8, 1.67 and 2a.I.9, 1.67 and 2a.1, 1.67 and 2a.2, 1.67 and 2a.3, 1.67 and 2a.4, 1.67 and 2a.5, 1.67 and 2a.6, 1.67 and 2a.7, 1.67 and 2a.8, 1.67 and 2a.9, 1.67 and 2a.10, 1.67 and 2a.11, 1.67 and 2a.12, 1.67 and 2a.13,
1.68 and 2a.I.1, 1.68 and 2a.I.2, 1.68 and 2a.I.3, 1.68 and 2a.I.4, 1.68 and 2a.I.5, 1.68 and 2a.I.6, 1.68 and 2a.I.7, 1.68 and 2a.I.8, 1.68 and 2a.I.9, 1.68 and 2a.1, 1.68 and 2a.2, 1.68 and 2a.3, 1.68 and 2a.4, 1.68 and 2a.5, 1.68 and 2a.6, 1.68 and 2a.7, 1.68 and 2a.8, 1.68 and 2a.9, 1.68 and 2a.10, 1.68 and 2a.11, 1.68 and 2a.12, 1.68 and 2a.13,
1.69 and 2a.I.1, 1.69 and 2a.I.2, 1.69 and 2a.I.3, 1.69 and 2a.I.4, 1.69 and 2a.I.5, 1.69 and 2a.I.6, 1.69 and 2a.I.7, 1.69 and 2a.I.8, 1.69 and 2a.I.9, 1.69 and 2a.1, 1.69 and 2a.2, 1.69 and 2a.3, 1.69 and 2a.4, 1.69 and 2a.5, 1.69 and 2a.6, 1.69 and 2a.7, 1.69 and 2a.8, 1.69 and 2a.9, 1.69 and 2a.10, 1.69 and 2a.11, 1.69 and 2a.12, 1.69 and 2a.13,
1.70 and 2a.I.1, 1.70 and 2a.I.2, 1.70 and 2a.I.3, 1.70 and 2a.I.4, 1.70 and 2a.I.5, 1.70 and 2a.I.6, 1.70 and 2a.I.7, 1.70 and 2a.I.8, 1.70 and 2a.I.9, 1.70 and 2a.1, 1.70 and 2a.2, 1.70 and 2a.3, 1.70 and 2a.4, 1.70 and 2a.5, 1.70 and 2a.6, 1.70 and 2a.7, 1.70 and 2a.8, 1.70 and 2a.9, 1.70 and 2a.10, 1.70 and 2a.11, 1.70 and 2a.12, 1.70 and 2a.13
1.71 and 2a.I.1, 1.71 and 2a.I.2, 1.71 and 2a.I.3, 1.71 and 2a.I.4, 1.71 and 2a.I.5, 1.71 and 2a.I.6, 1.71 and 2a.I.7, 1.71 and 2a.I.8, 1.71 and 2a.I.9, 1.71 and 2a.1, 1.71 and 2a.2, 1.71 and 2a.3, 1.71 and 2a.4, 1.71 and 2a.5, 1.71 and 2a.6, 1.71 and 2a.7, 1.71 and 2a.8, 1.71 and 2a.9, 1.71 and 2a.10, 1.71 and 2a.11, 1.71 and 2a.12, 1.71 and 2a.13,

The proportions in which the active substances 1 and 2a may be used in the active substance combinations according to the invention are variable. Active substances 1 and 2a may possibly be present in the form of salts, solvates or hydrates. Depending on the choice of the compounds 1 and 2a, the weight ratios which may be used within the scope of the present invention vary on the basis of the different molecular weights of the various salt forms. The pharmaceutical combinations according to the invention may contain 1 and 2a generally in ratios by weight ranging from 15 000:1 to 1:10, preferably from 6 000:1 to 10:1, e.g. 3 000:1 to 100:1.

The weight ratios specified hereinbefore and below are based on the free bases of the actives.

For example, without restricting the scope of the invention thereto, combinations of 1 and 2 according to the invention may contain the EGFR-inhibitor 1 and a beta-2 mimetic 2a in the following weight ratios: 15000:1, 14500:1, 14000:1, 13500:1, 13000:1, 12500:1, 12000:1, 11500:1, 11000:1, 10500:1, 10000:1, 9500:1, 9000:1, 8500:1, 8000:1, 7500:1, 7000:1, 6500:1, 6000:1, 5500:1, 5000:1, 4500:1, 4000:1, 3500:1, 3000:1, 2500:1, 2000:1, 1500:1, 1000:1, 900:1, 800:1, 700:1, 600:1, 500:1, 400:1, 300:1, 200:1.

The pharmaceutical compositions according to the invention containing the combinations of 1 and 2a are normally administered so that 1 and 2a are present together in doses of 5 to 15000 μg, preferably from 10 to 10000 μg, more preferably from 15 to 5000 μg, better still from 20 to 2000 μg per single dose.

For example, combinations of any of EGFR-inhibitors 1.1 to 1.71, especially those characterized as preferred hereinbefore and below, and 2a according to the invention contain a quantity of the actives such that the total dosage per single dose is about 100 μg, 105 μg, 110 μg, 115 μg, etc. (add stepwise 5 μg) up to 15000 μg.

It is clear to anyone skilled in the art that the suggested dosages per single dose specified above are not to be regarded as being limited to the numerical values actually stated. Fluctuations of about ±2.5 μg, particularly in the decimal range, are also included, as will be apparent to the skilled man. In these dosage ranges, the active substances 1 and 2a may be present in the weight ratios given above. For example, without restricting the scope of the invention thereto, the combinations in which any of the preferred EGFR inhibitors 1.1, 1.2, 1.8, 1.12, 1.13, 1.25, 1.26, 1.27, 1.28, 1.33, 1.45, 1.46, 1.47, 1.48, 1.62, 1.64, 1.67, 1.68, 1.69, 1.70 and 1.71 is used and in which 2a denotes any of the beta-2 mimetics 2a.I.1, 2a.I.2, 2a.I.3, 2a.I.4, 2a.I.5, 2a.I.6, 2a.I.7, 2a.1.8, 2a.I.9, 2a.1, 2a.2, 2a.3, 2a.4, 2a.5, 2a.6, 2a.7, 2a.8, 2a.9, 2a.10, 2a.11, 2a.12 and 2a.13, the pharmaceutical compositions according to the invention may contain for instance the following quantities for each single dose: 10 μg of 1 and 2.9 μg of 2a, 10 μg of 1 and 5.7 μg of 2a, 10 μg of 1 and 11.5 μg of 2a, 10 μg of 1 and 17.2 μg of 2a, 10 μg of 1 and 22.9 μg of 2a, 10 μg of 1 and 28.5 μg of 2a,

100 μg of 1 and 2.9 μg of 2a, 100 μg of 1 and 5.7 μg of 2a, 100 μg of 1 and 11.5 μg of 2a, 100 μg of 1 and 17.2 μg of 2a, 100 μg of 1 and 22.9 μg of 2a, 100 μg of 1 and 28.5 μg of 2a,
500 μg of 1 and 2.9 mg of 2a, 500 μg of 1 and 5.7 μg of 2a, 500 μg of 1 and 11.5 μg of 2a, 500 μg of 1 and 17.2 μg of 2a, 500 μg of 1 and 22.9 μg of 2a, 500 μg of 1 and 28.5 μg of 2a,
1000 μg of 1 and 2.9 μg of 2a, 1000 μg of 1 and 5.7 μg of 2a, 1000 μg of 1 and 11.5 μg of 2a, 1000 μg of 1 and 17.2 μg of 2a, 1000 μg of 1 and 22.9 μg of 2a, 1000 μg of 1 and 28.5 μg of 2a, 1000 μg of 1 and 2.9 μg of 2a,
2000 μg of 1 and 5.7 μg of 2a, 2000 μg of 1 and 11.5 μg of 2a, 2000 μg of 1 and 17.2 μg of 2a, 2000 μg of 1 and 22.9 μg of 2a, 2000 μg of 1 and 28.5 μg of 2a,
3000 μg of 1 and 5.7 μg of 2a, 3000 μg of 1 and 11.5 μg of 2a, 3000 μg of 1 and 17.2 μg of 2a, 3000 μg of 1 and 22.9 μg of 2a, 3000 μg of 1 and 28.5 μg of 2a,
4000 μg of 1 and 5.7 μg of 2a, 4000 μg of 1 and 11.5 μg of 2a, 4000 μg of 1 and 17.2 μg of 2a, 4000 μg of 1 and 22.9 μg of 2a, 4000 μg of 1 and 28.5 μg of 2a,
5000 μg of 1 and 5.7 μg of 2a, 5000 μg of 1 and 11.5 μg of 2a, 5000 μg of 1 and 17.2 μg of 2a, 5000 μg of 1 and 22.9 μg of 2a, 5000 μg of 1 and 28.5 μg of 2a,
6000 μg of 1 and 5.7 μg of 2a, 6000 μg of 1 and 11.5 μg of 2a, 6000 μg of 1 and 17.2 μg of 2a, 6000 μg of 1 and 22.9 μg of 2a, 6000 μg of 1 and 28.5 μg of 2a,
7000 μg of 1 and 5.7 μg of 2a, 7000 μg of 1 and 11.5 μg of 2a, 7000 μg of 1 and 17.2 μg of 2a, 7000 μg of 1 and 22.9 μg of 2a, 7000 μg of 1 and 28.5 μg of 2a,
8000 μg of 1 and 5.7 μg of 2a, 8000 μg of 1 and 11.5 μg of 2a, 8000 μg of 1 and 17.2 μg of 2a, 8000 μg of 1 and 22.9 μg of 2a, 8000 μg of 1 and 28.5 μg of 2a,
9000 μg of 1 and 5.7 μg of 2a, 9000 μg of 1 and 11.5 μg of 2a, 9000 μg of 1 and 17.2 μg of 2a, 9000 μg of 1 and 22.9 μg of 2a, 9000 μg of 1 and 28.5 μg of 2a,
10000 μg of 1 and 5.7 μg of 2a, 10000 μg of 1 and 11.5 μg of 2a, 10000 μg of 1 and 17.2 μg of 2a, 10000 μg of 1 and 22.9 μg of 2a, 10000 μg of 1 and 28.5 μg of 2a,
12500 μg of 1 and 5.7 μg of 2a, 12500 μg of 1 and 11.5 μg of 2a, 12500 μg of 1 and 17.2 μg of 2a, 12500 μg of 1 and 22.9 μg of 2a, 12500 μg of 1 and 28.5 μg of 2a,
15000 μg of 1 and 5.7 μg of 2a, 15000 μg of 1 and 11.5 μg of 2a, 15000 μg of 1 and 17.2 μg of 2a, 15000 μg of 1 and 22.9 μg of 2a, 15000 μg of 1 and 28.5 μg of 2a.

The pharmaceutical compositions according to the invention containing the combinations of 1 and 2a are usually administered so that 1 and 2a are present together in dosages of 100 μg to 100000 μg, preferably from 500 μg to 50000 μg, more preferably from 1000 μg to 10000 μg per single dose.

For example, combinations of any of EGFR-inhibitors 1.1 to 1.71, especially those characterized as preferred hereinbefore, and 2a according to the invention contain a quantity of the actives such that the total dosage per single dose is about 100 μg, 150 μg, 200 μg, 250 μg, etc. (add stepwise 50 μg) up to 50000 μg.

It is clear to anyone skilled in the art that the suggested dosages per single dose specified above are not to be regarded as being limited to the numerical values actually stated. Fluctuations of about ±2.5 μg, particularly in the decimal range, are also included, as will be apparent to the skilled man. In these dosage ranges, the active substances 1 and 2a may be present in the weight ratios given above.

One embodiment of the invention is a pharmaceutical composition comprising an EGFR kinase inhibitor 1 and a steroid 2b. Binary compositions containing only one active 1 and one active 2b, optionally together with one or more pharmaceutically acceptable excipients or carriers, are preferred. In the pharmaceutical combinations according to the invention preferred steroids 2b, which are optionally also referred to as corticosteroids, are selected from the group consisting of prednisolone (2b.1), etiprednole-dichloroacetate (2b.2), Etiprednole (2b.3), CP-4112 (2b.4), Loteprednol etabonate (213.5), Loteprednole (2b.6), NS-126 (2b.7), ST-26 (2b.8), NCX-1020 (2b.9) Betamethasone (21110), Deflazacorte (2b.11), 6α,9α-difluoro-11β-hydroxy-16α-methyl-3-oxo-17α-(2,2,3,3-tetramethylcyclopropylcarbonyl)oxy-androsta-1,4-diene-17β-carboxylic acid cyanomethyl ester (2b.12),6α,9α-Difluoro-11-hydroxy-16α-methyl-3-oxo-17α-propionyloxy-androsta-1,4-dien-17β-carbothion acid (S)-(2-oxo-tetrahydro-furan-3S-yl)ester (2b.13), Fluticasone proprionate (2b.14) Fluticasone furoate (2b.15), desciclesonide (2b.16), azmacort (2b.17), butoxocort propionat (2b.18), flumetasone (2b.19), mometasone furoate (2b.20) and beclomethasone dipropionate (2b.21). and optionally in the form of the salts and derivatives thereof, the solvates and/or hydrates thereof.

Preferably, the compound 213 is selected from among prednisolone (2b.1), etiprednole-dichloroacetate (2b.2), Etiprednole (2b.3), CP-4112 (2b.4), Loteprednol etabonate (2b.5), Loteprednole (2b.6), NS-126 (213.7), ST-26 (213.8), NCX-1020 (2b.9) Betamethasone (2b.10), Deflazacorte (2b.11),6α,9α-difluoro-11β-hydroxy-16α-methyl-3-oxo-17α-(2,2,3,3-tetramethylcyclopropylcarbonyl)oxy-androsta-1,4-diene-17β-carboxylic acid cyanomethyl ester (2b.12) and 6α,9α-Difluoro-11-hydroxy-16α-methyl-3-oxo-17α-propionyloxy-androsta-1,4-dien-17β-carbothion acid (S)-(2-oxo-tetrahydro-furan-3S-yl)ester (2b.13).

Especially preferred pharmaceutical compositions according to the invention comprise the following specific combinations of EGFR kinase inhibitors 1 and steroids 2b, either as free bases or pharmacologically acceptable acid addition salts:

1.1 and 2b.1, 1.1 and 2b.2, 1.1 and 2b.3, 1.1 and 2b.4, 1.1 and 2b.5, 1.1 and 2b.6, 1.1 and 2b.7, 1.1 and 213.8, 1.1 and 2b.9, 1.1 and 2b.10, 1.1 and 2b.11, 1.1 and 2b.12, 1.1 and 2b.13, 1.1 and 2b.14, 1.1 and 2b.15, 1.1 and 2b.16, 1.1 and 2b.17, 1.1 and 2b.18, 1.1 and 2b.19, 1.1 and 2b.20, 1.1 and 2b.21,
1.2 and 2b.1, 1.2 and 2b.2, 1.2 and 2b.3, 1.2 and 2b.4, 1.2 and 2b.5, 1.2 and 2b.6, 1.2 and 2b.7, 1.2 and 2b.8, 1.2 and 2b.9, 1.2 and 2b.10, 1.2 and 2b.11, 1.2 and 2b.12, 1.2 and 2b.13, 1.2 and 2b.14, 1.2 and 2b.15, 1.2 and 2b.16, 1.2 and 2b.17, 1.2 and 2b.18, 1.2 and 2b.19, 1.2 and 2b.20, 1.2 and 2b.21,
1.8 and 2b.1, 1.8 and 2b.2, 1.8 and 2b.3, 1.8 and 2b.4, 1.8 and 2b.5, 1.8 and 2b.6, 1.8 and 2b.7, 1.8 and 2b.8, 1.8 and 2b.9, 1.8 and 2b.10, 1.8 and 2b.11, 1.8 and 2b.12, 1.8 and 2b.13, 1.8 and 2b.14, 1.8 and 2b.15, 1.8 and 2b.16, 1.8 and 2b.17, 1.8 and 2b.18, 1.8 and 2b.19, 1.8 and 2b.20, 1.8 and 2b.21,
1.12 and 2b.1, 1.12 and 2b.2, 1.12 and 2b.3, 1.12 and 2b.4, 1.12 and 2b.5, 1.12 and 2b.6, 1.12 and 2b.7, 1.12 and 2b.8, 1.12 and 2b.9, 1.12 and 2b.10, 1.12 and 2b.11, 1.12 and 2b.12, 1.12 and 2b.13, 1.12 and 2b.14, 1.12 and 2b.15, 1.12 and to 2b.16, 1.12 and 2b17, 1.12 and 2b.18, 1.12 and 2b.19, 1.12 and 2b.20, 1.12 and 2b.21,
1.13 and 2b.1, 1.13 and 2b.2, 1.13 and 2b.3, 1.13 and 2b.4, 1.13 and 2b.5, 1.13 and 2b.6, 1.13 and 2b.7, 1.13 and 2b.8, 1.13 and 2b.9, 1.13 and 2b.10, 1.13 and 2b.11, 1.13 and 2b.12, 1.13 and 2b.13, 113 and 2b.14, 1.13 and 2b.15, 1.13 and 2b.16, 1.13 and 2b.17, 1.13 and 2b.18, 1.13 and 2b.19, 1.13 and 2b.20, 1.13 and 2b.21,
1.20 and 2b.1, 1.20 and 2b.2, 1.20 and 2b.3, 1.20 and 2b.4, 1.20 and 2b.5, 1.20 and 2b.6, 1.20 and 2b.7, 1.20 and 2b.8, 1.20 and 2b.9, 1.20 and 2b.10, 1.20 and 2b.11, 1.20 and 2b.12, 1.20 and 2b.13, 1.20, and 2b.14, 1.20 and 2b.15, 1.20 and 2b.16, 1.20 and 2b.17, 1.20 and 2b.18, 1.20 and 2b.19, 1.20 and 2b.20, 1.20 and 2b.21,
1.26 and 2b.1, 1.26 and 2b.2, 1.26 and 2b.3, 1.26 and 2b.4, 1.26 and 2b.5, 1.26 and 2b.6, 1.26 and 2b.7, 1.26 and 2b.8, 1.26 and 2b.9, 1.26 and 2b.10, 1.26 and 2b.11, 1.26 and 2b.12, 1.26 and 2b.13, 1.26 and 2b.14, 1.26 and 2b.15, 1.26 and 2b.16, 1.26 and 2b.17, 1.26 and 2b.18, 1.26 and 2b.19, 1.26 and 2b.20, 1.26 and 2b.21,
1.27 and 2b.1, 1.27 and 2b.2, 1.27 and 2b.3, 1.27 and 2b.4, 1.27 and 2b.5, 1.27 and 2b.6, 1.27 and 2b.7, 1.27 and 2b.8, 1.27 and 2b.9, 1.27 and 2b.10, 1.27 and 2b.11, 1.27 and 2b.12, 1.27 and 2b.13, 1.27 and 2b.14, 1.27 and 2b.15, 1.27 and 2b.16, 1.27 and 2b.17, 1.27 and 2b.18, 1.27 and 2b.19, 1.27 and 2b.20, 1.27 and 2b.21,
1.28 and 2b.1, 1.28 and 2b.2, 1.28 and 2b.3, 1.28 and 2b.4, 1.28 and 2b.5, 1.28 and 2b.6, 1.28 and 2b.7, 1.28 and 2b.8, 1.28 and 2b.9, 1.28 and 2b.10, 1.28 and 2b.11, 1.28 and 2b.12, 1.28 and 2b.13, 1.28 and 2b.14, 1.28 and 2b.15, 1.28 and 2b.16, 1.28 and 2b.17, 1.28 and 2b.18, 1.28 and 2b.19, 1.28 and 2b.20, 1.28 and 2b.21,
1.33 and 2b.1, 1.33 and 2b.2, 1.33 and 2b.3, 1.33 and 2b.4, 1.33 and 2b.5, 1.33 and 2b.6, 1.33 and 2b.7, 1.33 and 2b.8, 1.33 and 2b.9, 1.33 and 2b.10, 1.33 and 2b.11, 1.33 and 2b.12, 1.33 and 2b.13, 1.33 and 2b.14, 1.33 and 2b.15, 1.33 and 2b.16, 1.33 and 2b.17, 1.33 and 2b.18, 1.33 and 2b.19, 1.33 and 2b.20, 1.33 and 2b.21,
1.45 and 2b.1, 1.45 and 2b.2, 1.45 and 2b.3, 1.45 and 2b.4, 1.45 and 2b.5, 1.45 to and 2b.6, 1.45 and 2b.7, 1.45 and 2b.8, 1.45 and 2b.9, 1.45 and 2b.10, 1.45 and 2b.11, 1.45 and 2b.12, 1.45 and 2b.13, 1.45 and 2b.14, 1.45 and 2b.15, 1.45 and 2b.16, 1.45 and 2b.17, 1.45 and 2b.18, 1.45 and 2b.19, 1.45 and 2b.20, 1.45 and 2b.21
1.46 and 2b.1, 1.46 and 2b.2, 1.46 and 2b.3, 1.46 and 2b.4, 1.46 and 2b.5, 1.46 and 2b.6, 1.46 and 2b.7, 1.46 and 2b.8, 1.46 and 2b.9, 1.46 and 2b.10, 1.46 and 2b.11, 1.46 and 2b.12, 1.46 and 2b.13, 1.46 and 2b.14, 1.46 and 2b.15, 1.46 and 2b.16, 1.46 and 2b.17, 1.46 and 2b.18, 1.46 and 2b.19, 1.46 and 2b.20, 1.46 and 2b.21
1.47 and 2b.1, 1.47 and 2b.2, 1.47 and 2b.3, 1.47 and 2b.4, 1.47 and 2b.5, 1.47 and 2b.6, 1.47 and 2b.7, 1.47 and 2b.8, 1.47 and 2b.9, 1.47 and 2b.10, 1.47 and 2b.11, 1.47 and 2b.12, 1.47 and 2b.13, 1.47 and 2b.14, 1.47 and 2b.15, 1.47 and 2b.16, 1.47 and 2b.17, 1.47 and 2b.18, 1.47 and 2b.19, 1.47 and 2b.20, 1.47 and 2b.21,
1.48 and 2b.1, 1.48 and 2b.2, 1.48 and 2b.3, 1.48 and 2b.4, 1.48 and 2b.5, 1.48 and 2b.6, 1.48 and 2b.7, 1.48 and 2b.8, 1.48 and 2b.9, 1.48 and 2b.10, 1.48 and 2b.11, 1.48 and 2b.12, 1.48 and 2b.13, 1.48 and 2b.14, 1.48 and 2b.15, 1.48 and 2b.16, 1.48 and 2b.17, 1.48 and 2b.18, 1.48 and 2b.19, 1.48 and 2b.20, 1.48 and 2b.21,
1.62 and 2b.1, 1.62 and 2b.2, 1.62 and 2b.3, 1.62 and 2b.4, 1.62 and 2b.5, 1.62 and 2b.6, 1.62 and 2b.7, 1.62 and 2b.8, 1.62 and 2b.9, 1.62 and 2b.10, 1.62 and 2b.11, 1.62 and 2b.12, 1.62 and 2b.13, 1.62 and 2b.14, 1.62 and 2b.15, 1.62 and 2b.16, 1.62 and 2b.17, 1.62 and 2b.18, 1.62 and 2b.19, 1.62 and 2b.20, 1.62 and 2b.21,
1.64 and 2b.1, 1.64 and 2b.2, 1.64 and 2b.3, 1.64 and 2b.4, 1.64 and 2b.5, 1.64 and 2b.6, 1.64 and 2b.7, 1.64 and 2b.8, 1.64 and 2b.9, 1.64 and 2b.10, 1.64 and 2b.11, 1.64 and 2b.12, 1.64 and 2b.13, 1.64 and 2b.14, 1.64 and 2b.15, 1.64 and 2b.16, 1.64 and 2b.17, 1.64 and 2b.18, 1.64 and 2b.19, 1.64 and 2b.20, 1.64 and 2b.21,
1.67 and 2b.1, 1.67 and 2b.2, 1.67 and 2b.3, 1.67 and 2b.4, 1.67 and 2b.5, 1.67 and 2b.6, 1.67 and 2b.7, 1.67 and 2b.8, 1.67 and 2b.9, 1.67 and 2b.10, 1.67 and 2b.11, 1.67 and 2b.12, 1.67 and 2b.13, 1.67 and 2b.14, 1.67 and 2b.15, 1.67 and 2b.16, 1.67 and 2b.17, 1.67 and 2b.18, 1.67 and 2b.19, 1.67 and 2b.20, 1.67 and to 2b.21,
1.68 and 2b.1, 1.68 and 2b.2, 1.68 and 2b.3, 1.68 and 2b.4, 1.68 and 2b.5, 1.68 and 2b.6, 1.68 and 213.7, 1.68 and 2b.8, 1.68 and 2b.9, 1.68 and 2b.10, 1.68 and 2b.11, 1.68 and 2b.12, 1.68 and 2b.13, 1.68 and 2b.14, 1.68 and 2b.15, 1.68 and 2b.16, 1.68 and 2b.17, 1.68 and 2b.18, 1.68 and 2b.19, 1.68 and 2b.20, 1.68 and 2b.21,
1.69 and 2b.1, 1.69 and 2b.2, 1.69 and 2b.3, 1.69 and 2b.4, 1.69 and 2b.5, 1.69 and 2b.6, 1.69 and 2b.7, 1.69 and 2b.8, 1.69 and 2b.9, 1.69 and 2b.10, 1.69 and 2b.11, 1.69 and 2b.12, 1.69 and 2b.13, 1.69 and 2b.14, 1.69 and 2b.15, 1.69 and 2b.16, 1.69 and 2b.17, 1.69 and 2b.18, 1.69 and 2b.19, 1.69 and 2b.20, 1.69 and 2b.21
1.70 and 2b.1, 1.70 and 2b.2, 1.70 and 2b.3, 1.70 and 2b.4, 1.70 and 2b.5, 1.70 and 2b.6, 1.70 and 2b.7, 1.70 and 2b.8, 1.70 and 2b.9, 1.70 and 2b.10, 1.70 and 2b.11, 1.70 and 2b.12, 1.70 and 2b.13, 1.70 and 2b.14, 1.70 and 2b.15, 1.70 and 2b.16, 1.70 and 2b.17, 1.70 and 2b.18, 1.70 and 2b.19, 1.70 and 2b.20, 1.70 and 2b.21,
1.71 and 2b.1, 1.71 and 2b.2, 1.71 and 2b.3, 1.71 and 2b.4, 1.71 and 2b.5, 1.71 and 2b.6, 1.71 and 2b.7, 1.71 and 213.8, 1.71 and 2b.9, 1.71 and 213.10, 1.71 and 2b.11, 1.71 and 2b.12, 1.71 and 2b.13, 1.71 and 2b.14, 1.71 and 2b.15, 1.71 and 2b.16, 1.71 and 2b.17, 1.71 and 2b.18, 1.71 and 2b.19, 1.71 and 2b.20, 1.71 and 2b.21.

Especially preferred pharmaceutical compositions according to the invention comprise the following specific combinations of EGFR kinase inhibitors 1 and steroids 2b, either as free bases or pharmacologically acceptable acid addition salts:

Any reference to steroids 2b within the scope of the present invention includes a reference to the salts or derivatives which may be formed from the steroids. Examples of possible salts or derivatives include: sodium salts, sulphobenzoates, phosphates, isonicotinates, acetates, propionates, dihydrogen phosphates, palmitates, pivalates or furoates. In some cases the compounds of formula 2b may also occur in the form of their hydrates. Any reference to steroids 2b within the scope of the present invention also includes a reference to the compounds 2b in the form of their diastereomers, mixtures of diastereomers or in the form of the racemates.

The proportions in which the active substances 1 and 2b may be used in the active is substance combinations according to the invention are variable. Active substances 1 and 2b may possibly be present in the form of their solvates or hydrates. Depending on the choice of the compounds 1 and 2b, the weight ratios which may be used within the scope of the present invention vary on the basis of the different molecular weights of the various compounds and their different potencies.

As a rule, the pharmaceutical combinations according to the invention may contain the EGFR-inhibitor 1 and the steroid 2b in ratios by weight ranging from 5000:1 to 1:250, preferably from 2500:1 to 1:150, more preferably 1000:1 to 1:100, most preferred from 250:1 to 1:25.

For example, without restricting the scope of the invention thereto, preferred combinations according to the invention may contain an EGF kinase inhibitor 1 and any one of the steroids 2b, for example in the following ratios by weight (all based on free base): 500:1, 450:1, 400:1, 350:1, 300:1, 250:1, 200:1, 150:1, 100:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20:1:25, 1:30, 1:35, 1:40, 1:45, 1:50.

The pharmaceutical compositions according to the invention containing the combinations of 1 together with any one of the steroids 2b selected from preferably are administered so that 1 and the steroid 2b (values based on free base) are present together in dosages of 100 μg to 50000 μg, preferably from 500 μg to 25000 μg, more preferably from 2000 μg to 12000 μg per single dose.

For example, combinations of 1 and 2b according to the invention contain an amount of 1 and 2b (values based on free base) such that the total dosage per single dose is about 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 300 μg, 400 μg, 500 μg, 600 μg, 700 μg, 800 μg, 900 μg, 1000 μg, 1100 μg, 1200 μg, etc. (add stepwise 1000 μg) up to 50000 μg, or similar. It is clear to anyone skilled in the art that the suggested dosages per single dose specified above are not to be regarded as being limited to the numerical values actually stated. Fluctuations of is about ±2.5 μg, particularly in the decimal range, are also included, as will be apparent to the skilled man. In these dosage ranges, the active substances 1 and 2b may be present in the weight ratios given above.

For example, without restricting the scope of the invention thereto, the combinations of 1 and one of the steroids 2b may in particular contain a quantity of 1 and steroid 2b (values based on free base) such that, for each single dose,

100 μg of 1 and 25 μg of 2b, 100 μg of 1 and 50 μg of 2b, 100 μg of 1 and 75 μg of 2b, 100 μg of 1 and 100 μg of 2b, 100 μg of 1 and 125 μg of 2b, 100 μg of 1 and 150 μg of 2b, 100 μg of 1 and 200 μg of 2b, 100 μg of 1 and 250 μg of 2b,
200 μg of 1 and 25 μg of 2b, 200 μg of 1 and 50 μg of 2b, 200 μg of 1 and 75 μg of 2b, 200 μg of 1 and 100 μg of 2b, 200 μg of 1 and 125 μg of 2b, 200 μg of 1 and 150 μg of 2b, 200 μg of 1 and 200 μg of 2b, 200 μg of 1 and 250 μg of 2b,
500 μg of 1 and 25 μg of 2b, 500 μg of 1 and 50 μg of 2b, 500 μg of 1 and 75 μg of 2b, 500 μg of 1 and 100 μg of 2b, 500 μg of 1 and 125 μg of 2b, 500 μg of 1 and 150 μg of 2b, 500 μg of 1 and 200 μg of 2b, 500 μg of 1 and 250 μg of 2b,
1000 μg of 1 and 25 μg of 2b, 1000 μg of 1 and 50 μg of 2b, 1000 μg of 1 and 75 μg of 2b, 1000 μg of 1 and 100 μg of 2b, 1000 μg of 1 and 125 μg of 2b, 1000 μg of 1 and 150 μg of 2b, 1000 μg of 1 and 200 μg of 2b, 1000 μg of 1 and 250 μg of 2b,
5000 μg of 1 and 25 μg of 2b, 5000 μg of 1 and 50 μg of 2b, 5000 μg of 1 and 75 μg of 2b, 5000 μg of 1 and 100 μg of 2b, 5000 μg of 1 and 125 μg of 2b, 5000 μg of 1 and 150 μg of 2b, 5000 μg of 1 and 200 μg of 2b, 5000 μg of 1 and 250 μg of 2b,
10000 μg of 1 and 25 μg of 2b, 10000 μg of 1 and 50 μg of 2, 10000 μg of 1 and 75 μg of 2b, 10000 μg of 1 and 10000 μg of 2b, 10000 μg of 1 and 125 μg of 2b, 10000 μg of 1 and 150 μg of 2b, 10000 μg of 1 and 200 μg of 2b, 10000 μg of 1 and 250 μg of 2b,
25000 μg of 1 and 25 μg of 2b, 25000 μg of 1 and 50 μg of 2b, 25000 μg of 1 and 75 μg of 2b, 25000 μg of 1 and 100 μg of 2b, 25000 μg of 1 and 125 μg of 2b, 25000 μg of 1 and 150 μg of 2b, 25000 μg of 1 and 200 μg of 2b, 25000 μg of 1 and 250 μg of 2b,
50000 μg of 1 and 25 μg of 2b, 50000 μg of 1 and 50 μg of 2b, 50000 μg of 1 and 75 μg of 2b, 50000 μg of 1 and 100 μg of 2b, 50000 μg of 1 and 125 μg of 2b, 50000 μg of and 150 μg of 2b, 50000 μg of 1 and 200 μg of 2b, 50000 μg of 1 and 250 μg of 2b.

From the aforementioned examples for suitable doses of the 1 and 2b containing combinations according to the invention, the corresponding amounts of the preferably used acid addition salts of 1 and 2b are readily calculable.

One embodiment of the invention is a pharmaceutical composition comprising an EGFR kinase inhibitor 1 and a PDE IV inhibitor 2c. Binary compositions containing only one active 1 and one active 2c, optionally together with one or more pharmaceutically acceptable excipients or carriers, are preferred. In the pharmaceutical combinations according to the invention preferred PDE IV inhibitors 2c are selected from the group consisting of oglemilast 2c.1, tofimilast 2c.2, pumafentrine 2c.3 and lirimilaste 2c.4, optionally in the form of the racemates, the enantiomers, the diastereomers and optionally the pharmacologically acceptable acid addition salts thereof, and the hydrates thereof.

optionally in the form of the racemates, the enantiomers, the diastereomers and optionally the pharmacologically acceptable acid addition salts thereof, and the hydrates thereof.

In the pharmaceutical compositions according to the invention, the compounds 2c may be present in the form of their racemates, enantiomers or mixtures thereof. The separation of the enantiomers from the racemates may be carried out using methods known in the art (e.g. by chromatography on chiral phases, etc.).

Especially preferred pharmaceutical compositions according to the invention comprise the following specific combinations of EGFR kinase inhibitors 1 and PDE IV inhibitors 2c, either as free bases or pharmacologically acceptable acid addition salts:

1.1 and 2c.1, 1.1 and 2c.2, 1.1 and 2c.3, 1.1 and 2c.4, 1.2 and 2c.1, 1.2 and 2c.2, 1.2 and 2c.3, 1.2 and 2c.4, 1.8 and 2c.1, 1.8 and 2c.2, 1.8 and 2c.3, 1.8 and 2c.4, 1.12 and 2c.1, 1.12 and 2c.2, 1.12 and 2c.3, 1.12 and 2c.4, 1.13 and 2c.1, 1.13 and 2c.2, 1.13 and 2c.3, 1.13 and 2c.4, 1.20 and 2c.1, 1.20 and 2c.2, 1.20 and 2c.3, 1.20 and 2c.4, 1.26 and 2c.1, 1.26 and 2c.2, 1.26 and 2c.3, 1.26 and 2c.4, 1.27 and 2c.1, 1.27 and 2c.2, 1.27 and 2c.3, 1.27 and 2c.4, 1.28 and 2c.1, 1.28 and 2c.2, 1.28 and 2c.3, 1.28 and 2c.4, 1.33 and 2c.1, 1.33 and 2c.2, 1.33 and 2c.3, 1.33 and 2c.4, 1.45 and 2c.1, 1.45 and 2c.2, 1.45 and 2c.3, 1.45 and 2c.4, 1.46 and 2c.1, 1.46 and 2c.2, 1.46 and 2c.3, 1.46 and 2c.4, 1.47 and 2c.1, 1.47 and 2c.2, 1.47 and 2c.3, 1.47 and 2c.4, 1.48 and 2c.1, 1.48 and 2c.2, 1.48 and 2c.3, 1.48 and 2c.4, 1.62 and 2c.1, 1.62 and 2c.2, 1.62 and 2c.3, 1.62 and 2c.4, 1.64 and 2c.1, 1.64 and 2c.2, 1.64 and 2c.3, 1.64 and 2c.4, 1.67 and 2c.1, 1.67 and 2c.2, 1.67 and 2c.3, 1.67 and 2c.4, 1.68 and 2c.1, 1.68 and 2c.2, 1.68 and 2c.3, 1.68 and 2c.4, 1.69 and 2c.1, 1.69 and 2c.2, 1.69 and 2c.3, 1.69 and 2c.4, 1.70 and 2c.1, 1.70 and 2c.2, 1.70 and 2c.3, 1.70 and 2c.4, 1.71 and 2c.1, 1.71 and 2c.2, 1.71 and 2c.3, 1.71 and 2c.4.

The proportions in which the active substances 1 and 2c may be used in the active substance combinations according to the invention are variable. Active substances and 2c may possibly be present in the form of their solvates or hydrates. Depending on the choice of the compounds 1 and 2c, the weight ratios which may be used within the scope of the present invention vary on the basis of the different molecular weights of the various salt forms.

As a rule, the pharmaceutical combinations according to the invention may contain compounds 1 and 2c in ratios by weight ranging from 10000:1 to 1:500, preferably from 4000:1 to 1:100, more preferred from 2000:1 to 1:50, most preferred 1000:1 to 1:20. For example, without restricting the scope of the invention thereto, preferred combinations may contain 1 and PDE-IV inhibitor 2c in the following weight ratios: 4000:1, 3900:1, 3800:1, 3700:1, 3600:1, 3500:1, 3400:1, 3300:1, 3200:1, 3100:1, 3000:1, 2900:1, 2800:1 2700:1, 2600:1, 2500:1, 2400:1, 2300:1, 2200:1 2100:1, 2000:1, 1900:1, 1800:1, 1700:1, 1600:1, 1500:1, 1400:1, 1300:1, 1200:1, 1100:1, 1000:1, 900:1, 800:1, 700:1, 600:1, 500:1, 400:1, 300:1, 200:1, 100:1, 90:1, 80:1, 70:1, 60:1, 50:1, 40:1, 35:1, 30:1, 25:1, 20:1.

The pharmaceutical compositions according to the invention containing the combinations of 1 and 2c are normally administered so that 1 and 2c are present together in doses of 1 to 100000 μg, preferably from 10 to 50000 μg, more preferably from 100 to 25000 μg, better still from 500 to 10000 μg per single dose. For example, combinations of 1 and 2c according to the invention contain a quantity of 1 and PDE-IV inhibitor 2c such that the total dosage per single dose is about 5 μg, 10 μg, 15 μg, 20 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 300 μg, 400 μg, 500 μg, 600 μg, etc. (add stepwise 100 μg) up to 50000 μg, or similar.

The suggested dosages per single dose specified above are not to be regarded as being limited to the numerical values actually stated, but are intended as dosages which are disclosed by way of example. Of course, dosages which may fluctuate about the abovementioned numerical values within a range of about +/−2.5 μg are also included in the values given above by way of example. In these dosage ranges, the active substances 1 and 2c may be present in the weight ratios given above.

For example, without restricting the scope of the invention thereto, the combinations of 1 and 2c according to the invention may contain a quantity of 1 and PDE-IV inhibitor 2 in such an amount that the following quantities of the active substances are administered per single dose:

100 μg of 1 and 25 μg of 2c, 100 μg of 1 and 50 μg of 2c, 100 μg of 1 and 100 μg of 2c, 100 μg of 1 and 200 μg of 2c, 100 μg of 1 and 300 μg of 2c, 100 μg of 1 and 400 μg of 2c, 100 μg of 1 and 500 μg of 2c, 100 μg of 1 and 600 μg of 2c, 100 μg of 1 and 700 μg of 2c, 100 μg of 1 and 800 μg of 2c, 100 μg of 1 and 900 μg of 2c, 100 μg of 1 and 1000 μg of 2c, 100 μg of 1 and 1250 μg of 2c, 100 μg of 1 and 1500 μg of 2c, 100 μg of 1 and 1750 μg of 2c, 100 μg of 1 and 2000 μg of 2c,
200 μg of 1 and 25 μg of 2c, 200 μg of 1 and 50 μg of 2c, 200 μg of 1 and 100 μg of 2c, 200 μg of 1 and 200 μg of 2c, 200 μg of 1 and 300 μg of 2c, 200 μg of 1 and 400 μg of 2c, 200 μg of 1 and 500 μg of 2c, 200 μg of 1 and 600 μg of 2c, 200 μg of 1 and 700 μg of 2c, 200 μg of 1 and 800 μg of 2c, 200 μg of 1 and 900 μg of 2c, 200 μg of 1 and 1000 μg of 2c, 200 μg of 1 and 1250 μg of 2c, 200 μg of 1 and 1500 μg of 2c, 200 μg of 1 and 1750 μg of 2c, 200 μg of 1 and 2000 μg of 2c,
500 μg of 1 and 25 μg of 2c, 500 μg of 1 and 50 μg of 2c, 500 μg of 1 and 100 μg of 2c, 500 μg of 1 and 200 μg of 2c, 500 μg of 1 and 300 μg of 2c, 500 μg of 1 and 400 μg of 2c, 500 μg of 1 and 500 μg of 2c, 500 μg of 1 and 600 μg of 2c, 500 μg of 1 and 700 μg of 2c, 500 μg of 1 and 800 μg of 2c, 500 μg of 1 and 900 μg of 2c, 500 μg of 1 and 1000 μg of 2c, 500 μg of 1 and 1250 μg of 2c, 500 μg of 1 and 1500 μg of 2c, 500 μg of 1 and 1750 μg of 2c, 500 μg of 1 and 2000 μg of 2c,
1000 μg of 1 and 25 μg of 2c, 1000 μg of 1 and 50 μg of 2c, 1000 μg of 1 and 100 μg of 2c, 1000 μg of 1 and 200 μg of 2c, 1000 μg of 1 and 300 μg of 2c, 1000 μg of 1 and 400 μg of 2c, 1000 μg of 1 and 500 μg of 2c, 1000 μg of 1 and 600 μg of 2c, 1000 μg of 1 and 700 μg of 2c, 1000 μg of 1 and 800 μg of 2c, 1000 μg of 1 and 900 μg of 2c, 1000 μg of 1 and 1000 μg of 2c, 1000 μg of 1 and 1250 μg of 2c, 1000 μg of 1 and 1500 μg of 2c, 1000 μg of 1 and 1750 μg of 2c, 1000 μg of 1 and 2000 μg of 2c,
5000 μg of 1 and 25 μg of 2c, 5000 μg of 1 and 50 μg of 2c, 5000 μg of 1 and 100 μg of 2c, 5000 μg of 1 and 200 μg of 2c, 5000 μg of 1 and 300 μg of 2c, 5000 μg of 1 and 400 μg of 2c, 5000 μg of 1 and 500 μg of 2c, 5000 μg of 1 and 600 μg of 2c, 5000 μg of 1 and 700 μg of 2c, 5000 μg of 1 and 800 μg of 2c, 5000 μg of 1 and 900 μg of 2c, 5000 μg of 1 and 1000 μg of 2c, 5000 μg of 1 and 1250 μg of 2c, 5000 μg of 1 and 1500 μg of 2c, 5000 μg of 1 and 1750 μg of 2c, 5000 μg of 1 and 2000 μg of 2c,
10000 μg of 1 and 25 μg of 2c, 10000 μg of 1 and 50 μg of 2c, 10000 μg of 1 and 100 μg of 2c, 10000 μg of 1 and 200 μg of 2c, 10000 μg of 1 and 300 μg of 2c, 10000 μg of 1 and 400 μg of 2c, 10000 μg of 1 and 500 μg of 2c, 10000 μg of 1 and 600 μg of 2c, 10000 μg of 1 and 700 μg of 2c, 10000 μg of 1 and 800 μg of 2c, 10000 μg of 1 and 900 μg of 2c, 10000 μg of 1 and 1000 μg of 2c, 10000 μg of 1 and 1250 μg of 2c, 10000 μg of 1 and 1500 μg of 2c, 10000 μg of 1 and 1750 μg of 2c, 10000 μg of 1 and 2000 μg of 2c,
25000 μg of 1 and 25 μg of 2c, 25000 μg of 1 and 50 μg of 2c, 25000 μg of 1 and 100 μg of 2c, 25000 μg of 1 and 200 μg of 2c, 25000 μg of 1 and 300 μg of 2c, 25000 μg of 1 and 400 μg of 2c, 25000 μg of 1 and 500 μg of 2c, 25000 μg of 1 and 600 μg of 2c, 25000 μg of 1 and 700 μg of 2c, 25000 μg of 1 and 800 μg of 2c, 25000 μg of 1 and 900 μg of 2c, 25000 μg of 1 and 1000 μg of 2c, 25000 μg of 1 and 1250 μg of 2c, 25000 μg of 1 and 1500 μg of 2c, 25000 μg of 1 and 1750 μg of 2c, 25000 μg of 1 and 2000 μg of 2c,
50000 μg of 1 and 25 μg of 2c, 50000 μg of 1 and 50 μg of 2c, 50000 μg of 1 and 100 μg of 2c, 50000 μg of 1 and 200 μg of 2c, 50000 μg of 1 and 300 μg of 2c, 50000 μg of 1 and 400 μg of 2c, 50000 μg of 1 and 500 μg of 2c, 50000 μg of 1 and 600 μg of 2c, 50000 μg of 1 and 700 μg of 2c, 50000 μg of 1 and 800 μg of 2c, 50000 μg of 1 and 900 μg of 2c, 50000 μg of 1 and 1000 μg of 2c, 50000 μg of 1 and 1250 μg of 2c, 50000 μg of 1 and 1500 μg of 2c, 50000 μg of 1 and 1750 μg of 2c, 50000 μg of 1 and 2000 μg of 2c.

One embodiment of the invention is a pharmaceutical composition comprising an EGFR kinase inhibitor 1 and a p38 MAP kinase inhibitor 2d. Binary compositions containing only one active 1 and one active 2d, optionally together with one or more pharmaceutically acceptable excipients or carriers, are preferred.

p38 kinase inhibitors applicable within the scope of the invention are known in the art. Within the scope of the present invention the term p38 kinase inhibitors 2d denotes compounds selected from the group consisting of TAK-715 (2d.1), VX-745 (2d.2), HEP-689 (2d.3), PS-540446 (2d.4), RWJ-67657 (2d.5), SB-220025 (2d.6), AMG-548 (2d.7), Ro-320-1195 (2d.8), SCID-323 (2d.9), 2-(2-Isopropylamino-1,1-dimethyl-ethylamino)-3-methyl-5-naphthalen-2-yl-6-pyridin-4-yl-3H-pyrimidin-4-one (2d.10), 6-[2-tert-Butyl-5-(2,4-difluoro-phenyl)-1H-imidazol-4-yl]-1-(2-methyl-propane-2-sulfonyl)-1H-imidazo[4,5-b]pyridin-2-ylamine (2d.11), 3-(2-Chloro-phenyl)-7-(tetrahydro-pyran-4-ylamino)-1H-[1,6]naphthyridin-2-one (2d.12), 2-Phenyl-3-[2-(1-phenykethylamino)-pyrimidin-4-yl]-imidazo[1,2-a]pyrimidin-7-ylamine (2d.13), 1-{4-[5-(4-Chloro-2-fluoro-phenyl)-4-pyrimidin-4-yl-2H-pyrazol-3-yl]-piperidin-1-yl}-2-hydroxy-ethanone (2d.14), 2-(2-Isopropylamino-1,1-dimethyl-ethylamino),-3-methyl-5-naphthalen-2-yl-6-pyridin-4-yl-3H-pyrimidin-4-one (2d.15), [5-(4-Methoxy-phenyl)-4-(3-trifluoromethyl-phenyl)-4H-[1,2,4]triazol-3-ylsulfanyl]-acetic acid benzyl ester (2d.16), 3-Fluoro-N-[4-methyl-3-(2-methylsulfanyl-pyrimidin-4-ylamino)-phenyl]-5-morpholin-4-yl-benzamide (2d.17), 5-tert-Butyl-3-[3-(2,3-dichloro-phenyl)-ureido]-1H-pyrrole-2-carboxylic acid methyl ester (2d.18), 6-[2-tert-Butyl-5-(2,4-difluoro-phenyl)-1H-imidazol-4-yl]-1-(2-methyl-propane-2-sulfonyl),-1H-imidazo[4,5-b]pyridin-2-ylamine (2d.19), 4-[4-(4-Fluoro-phenyl)-5-(2-methoxy-pyrimidin-4-yl)-imidazol-1-yl]-cyclohexapol (2d.20), 2-(2,4-Dimethyl-phenoxy)-4-[5-(4-fluoro-phenyl)-3-piperidin-4-yl-3H-imidazol-4-yl]-pyrimidine (2d.21),[2-Chloro-4-(4-fluoro-2-methyl-phenylamino)-phenyl]-o-tolyl-methanone (2d.22), N-(2-Methoxy-benzyl)-4-phenoxy-benzamide (2d.23), 7-(1-tert-Butyl-piperidin-4-yl)-5-(2-chloro-4-fluoro-phenyl)-1-(2,6-dichloro-phenyl)-3,4-dihydro-1H-quinazolin-2-one (2d.24), {4-[5-(4-Fluoro-phenyl)-2-methylsulfanyl-3H-imidazol-4-yl]-pyridin-2-yl}-(1-phenyl-ethyl)-amine (2d.25), 4-(3,4-Dichloro-phenyl)-5-pyridin-4-yl-thiazol-2-ylamine (2d.26), 4-[4-(4-Fluoro-phenyl)-5-pyridin-4-yl-oxazol-2-yl]-1-methyl-piperidin-4-ol (2d.27), {2-[5-[2-(Cyclopropylmethyl-amino)-pyrimidin-4-yl]-4-(4-fluoro-phenyl)-1H-imidazol-2-yl]-5-methyl-[1,3]dioxan-5-yl}-(4-methyl-piperazin-1-0)-methanone (2d.28)

optionally in the form of the racemates, the enantiomers, the diastereomers and optionally the pharmacologically acceptable acid addition salts thereof, and the hydrates thereof;
Any reference to the abovementioned p38 kinase inhibitors 2d within the scope of the present invention includes a reference to any pharmaceutically acceptable acid addition salts thereof which may exist. By the physiologically or pharmaceutically acceptable acid addition salts which may be formed from 2d are meant, according to the invention, pharmaceutically acceptable salts selected from among the salts of hydrochloric, hydrobromic, sulfuric, nitric, perchioric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids.

Any reference to the abovementioned p38 kinase inhibitors 2d within the scope of the present invention includes a reference to any alkali metal and alkaline earth metal salts thereof which may exist. If the compounds 2d are present in the form of their basic salts, the sodium or potassium salts are particularly preferred.

The pharmaceutical combinations of 1 and 2d according to the invention are preferably administered by parenteral or oral route or by inhalation, the latter being particularly preferred. For oral or parenteral administration the pharmaceutical compositions according to the invention may be administered e.g. in the form of solutions and tablets. For inhalation, as preferred according to the invention, suitable inhalable powders may be used which are packed into suitable capsules (inhalettes) and administered using suitable powder inhalers. Alternatively, the drug may be inhaled by the application of suitable inhalation aerosols. These include inhalation aerosols which contain HFA134a, HFA227 or a mixture thereof as propellant gas. The drug may also be inhaled using suitable solutions of the pharmaceutical combination consisting of 1 and 2d.

Especially preferred pharmaceutical compositions according to the invention comprise the following specific combinations of EGFR kinase inhibitors 1 and p38 kinase inhibitors 2d, either as free bases or pharmacologically acceptable acid addition salts

1.1 and 2d.1, 1.1 and 2d.2, 1.1 and 2d.3, 1.1 and 2d.4, 1.1 and 2d.5, 1.1 and 2d.6, 1.1 and 2d.7, 1.1 and 2d.8, 1.1 and 2d.9, 1.1 and 2d.10, 1.1 and 2d.11, 1.1 and 2d.12, 1.1 and 2d.13, 1.1 and 2d.14, 1.1 and 2d.15, 1.1 and 2d.16, 1.1 and 2d.17, 1.1 and 2d.18, 1.1 and 2d.19, 1.1 and 2d.20, 1.1 and 2d.21, 1.1 and 2d.22, 1.1 and 2d.23, 1.1 and 2d.24, 1.1 and 2d.25, 1.1 and 2d.26, 1.1 and 2d.27, 1.1 and 2d.28,
1.2 and 2d.1, 1.2 and 2d.2, 1.2 and 2d.3, 1.2 and 2d.4, 1.2 and 2d.5, 1.2 and 2d.6, 1.2 and 2d.7, 1.2 and 2d.8, 1.2 and 2d.9, 1.2 and 2d.10, 1.2 and 2d.11, 1.2 and 2d.12, 1.2 and 2d.13, 1.2 and 2d.14, 1.2 and 2d.15, 1.2 and 2d.16, 1.2 and 2d.17, 1.2 and 2d.18, 1.2 and 2d.19, 1.2 and 2d.20, 1.2 and 2d.21, 1.2 and 2d.22, 1.2 and 2d.23, 1.2 and 2d.24, 1.2 and 2d.25, 1.2 and 2d.26, 1.2 and 2d.27, 1.2 and 2d.28,
1.8 and 2d.1, 1.8 and 2d.2, 1.8 and 2d.3, 1.8 and 2d.4, 1.8 and 2d.5, 1.8 and 2d.6, 1.8 and 2d.7, 1.8 and 2d.8, 1.8 and 2d.9, 1.8 and 2d.10, 1.8 and 2d.11, 1.8 and 2d.12, 1.8 and 2d.13, 1.8 and 2d.14, 1.8 and 2d.15, 1.8 and 2d.16, 1.8 and 2d.17, 1.8 and 2d.18, 1.8 and 2d.19, 1.8 and 2d.20, 1.8 and 2d.21, 1.8 and 2d.22, 1.8 and 2d.23, 1.8 and 2d.24, 1.8 and 2d.25, 1.8 and 2d.26, 1.8 and 2d.27, 1.8 and 2d.28,
1.12 and 2d.1, 1.12 and 2d.2, 1.12 and 2d.3, 1.12 and 2d.4, 1.12 and 2d.5, 1.12 to and 2d.6, 1.12 and 2d.7, 1.12 and 2d.8, 1.12 and 2d.9, 1.12 and 2d.10, 1.12 and 2d.11, 1.12 and 2d.12, 1.12 and 2d.13, 1.12 and 2d.14, 1.12 and 2d.15, 1.12 and 2d.16, 1.12 and 2d.17, 1.12 and 2d.18, 1.12 and 2d.19, 1.12 and 2d.20, 1.12 and 2d.21, 1.12 and 2d.22, 1.12 and 2d.23, 1.12 and 2d.24, 1.12 and 2d.25, 1.12 and 2d.26, 1.12 and 2d.27, 1.12 and 2d.28,
1.13 and 2d.1, 1.13 and 2d.2, 1.13 and 2d.3, 1.13 and 2d.4, 1.13 and 2d.5, 1.13 and 2d.6, 1.13 and 2d.7, 1.13 and 2d.8, 1.13 and 2d.9, 1.13 and 2d.10, 1.13 and 2d.11, 1.13 and 2d.12, 1.13 and 2d.13, 1.13 and 2d.14, 1.13 and 2d.15, 1.13 and 2d.16, 1.13 and 2d.17, 1.13 and 2d.18, 1.13 and 2d.19, 1.13 and 2d.20, 1.13 and 2d.21, 1.13 and 2d.22, 1.13 and 2d.23, 1.13 and 2d.24, 1.13 and 2d.25, 1.13 and 2d.26, 1.13 and 2d.27, 1.13 and 2d.28,
1.20 and 2d.1, 1.20 and 2d.2, 1.20 and 2d.3, 1.20 and 2d.4, 1.20 and 2d.5, 1.20 and 2d.6, 1.20 and 2d.7, 1.20 and 2d.8, 1.20 and 2d.9, 1.20 and 2d.10, 1.20 and 2d.11, 1.20 and 2d.12, 1.20 and 2d.13, 1.20 and 2d.14, 1.20 and 2d.15, 1.20 and 2d.16, 1.20 and 2d.17, 1.20 and 2d.18, 1.20 and 2d.19, 1.20 and 2d.20, 1.20 and 2d.21, 1.20 and 2d.22, 1.20 and 2d.23, 1.20 and 2d.24, 1.20 and 2d.25, 1.20 and 2d.26, 1.20 and 2d.27, 1.20 and 2d.28,
1.26 and 2d.1, 1.26 and 2d.2, 1.26 and 2d.3, 1.26 and 2d.4, 1.26 and 2d.5, 1.26 and 2d.6, 1.26 and 2d.7, 1.26 and 2d.8, 1.26 and 2d.9, 1.26 and 2d.10, 1.26 and 2d.11, 1.26 and 2d.12, 1.26 and 2d.13, 1.26 and 2d.14, 1.26 and 2d.15, 1.26 and 2d.16, 1.26 and 2d.17, 1.26 and 2d.18, 1.26 and 2d.19, 1.26 and 2d.20, 1.26 and 2d.21, 1.26 and 2d.22, 1.26 and 2d.23, 1.26 and 2d.24, 1.26 and 2d.25, 1.26 and 2d.26, 1.26 and 2d.27, 1.26 and 2d.28,
1.27 and 2d.1, 1.27 and 2d.2, 1.27 and 2d.3, 1.27 and 2d.4, 1.27 and 2d.5, 1.27 and 2d.6, 1.27 and 2d.7, 1.27 and 2d.8, 1.27 and 2d.9, 1.27 and 2d.10, 127 and 2d.11, 1.27 and 2d.12, 1.27 and 2d.13, 1.27 and 2d.14, 1.27 and 2d.15, 1.27 and 2d.16, 1.27 and 2d.17, 1.27 and 2d.18, 1.27 and 2d.19, 1.27 and 2d.20, 1.27 and 2d.21, 1.27 and 2d.22, 1.27 and 2d.23, 1.27 and 2d.24, 1.27 and 2d.25, 1.27 and 2d.26, 1.27 and 2d.27, 1.27 and 2d.28,
1.28 and 2d.1, 1.28 and 2d.2, 1.28 and 2d.3, 1.28 and 2d.4, 1.28 and 2d.5, 1.28 and 2d.6, 1.28 and 2d.7, 1.28 and 2d.8, 1.28 and 2d.9, 1.28 and 2d.10, 1.28 and 2d.11, 1.28 and 2d.12, 1.28 and 2d.13, 1.28 and 2d.14, 1.28 and 2d.15, 1.28 and 2d.16, 1.28 and 2d.17, 1.28 and 2d.18, 1.28 and 2d.19, 1.28 and 2d.20, 1.28 and 2d.21, 1.28 and 2d.22, 1.28 and 2d.23, 1.28 and 2d.24, 1.28 and 2d.25, 1.28 and 2d.26, 1.28 and 2d.27, 1.28 and 2d.28,
1.33 and 2d.1, 1.33 and 2d.2, 1.33 and 2d.3, 1.33 and 2d.4, 1.33 and 2d.5, 1.33 and 2d.6, 1.33 and 2d.7, 1.33 and 2d.8, 1.33 and 2d.9, 1.33 and 2d.10, 1.33 and is 2d.11, 1.33 and 2d.12, 1.33 and 2d.13, 1.33 and 2d.14, 1.33 and 2d.15, 1.33 and 2d.16, 1.33 and 2d.17, 1.33 and 2d.18, 1.33 and 2d.19, 1.33 and 2d.20, 1.33 and 2d.21, 1.33 and 2d.22, 1.33 and 2d.23, 1.33 and 2d.24, 1.33 and 2d.25, 1.33 and 2d.26, 1.33 and 2d.27, 1.33 and 2d.28,
1.45 and 2d.1, 1.45 and 2d.2, 1.45 and 2d.3, 1.45 and 2d.4, 1.45 and 2d.5, 1.45 and 2d.6, 1.45 and 2d.7, 1.45 and 2d.8, 1.45 and 2d.9, 1.45 and 2d.10, 1.45 and 2d.11, 1.45 and 2d.12, 1.45 and 2d.13, 1.45 and 2d.14, 1.45 and 2d.15, 1.45 and 2d.16, 1.45 and 2d.17, 1.45 and 2d.18, 1.45 and 2d.19, 1.45 and 2d.20, 1.45 and 2d.21, 1.45 and 2d.22, 1.45 and 2d.23, 1.45 and 2d.24, 1.45 and 2d.25, 1.45 and 2d.26, 1.45 and 2d.27, 1.45 and 2d.28,
1.46 and 2d.1, 1.46 and 2d.2, 1.46 and 2d.3, 1.46 and 2d.4, 1.46 and 2d.5, 1.46 and 2d.6, 1.46 and 2d.7, 1.46 and 2d.8, 1.46 and 2d.9, 1.46 and 2d.10, 1.46 and 2d.11, 1.46 and 2d.12, 1.46 and 2d.13, 1.46 and 2d.14, 1.46 and 2d.15, 1.46 and 2d.16, 1.46 and 2d.17, 1.46 and 2d.18, 1.46 and 2d.19, 1.46 and 2d.20, 1.46 and 2d.21, 1.46 and 2d.22, 1.46 and 2d.23, 1.46 and 2d.24, 1.46 and 2d.25, 1.46 and 2d.26, 1.46 and 2d.27, 1.46 and 2d.28,
1.47 and 2d.1, 1.47 and 2d.2, 1.47 and 2d.3, 1.47 and 2d.4, 1.47 and 2d.5, 1.47 and 2d.6, 1.47 and 2d.7, 1.47 and 2d.8, 1.47 and 2d.9, 1.47 and 2d.10, 1.47 and 2d.11, 1.47 and 2d.12, 1.47 and 2d.13, 1.47 and 2d.14, 1.47 and 2d.15, 1.47 and 2d.16, 1.47 and 2d.17, 1.47 and 2d.18, 1.47 and 2d.19, 1.47 and 2d.20, 1.47 and 2d.21, 1.47 and 2d.22, 1.47 and 2d.23, 1.47 and 2d.24, 1.47 and 2d.25, 1.47 and 2d.26, 1.47 and 2d.27, 1.47 and 2d.28, 1.48 and 2d.1, 1.48 and 2d.2, 1.48 and 2d.3, 1.48 and 2d.4, 1.48 and 2d.5, 1.48 and 2d.6, 1.48 and 2d.7, 1.48 and 2d.8, 1.48 and 2d.9, 1.48 and 2d.10, 1.48 and 2d.11, 1.48 and 2d.12, 1.48 and 2d.13, 1.48 and 2d.14, 1.48 and 2d.15, 1.48 and 2d.16, 1.48 and 2d.17, 1.48 and 2d.18, 1.48 and 2d.19, 1.48 and 2d.20, 1.48 and 2d.21, 1.48 and 2d.22, 1.48 and 2d.23, 1.48 and 2d.24, 1.48 and 2d.25, 1.48 and 2d.26, 1.48 and 2d.27, 1.48 and 2d.28,
1.62 and 2d.1, 1.62 and 2d.2, 1.62 and 2d.3, 1.62 and 2d.4, 1.62 and 2d.5, 1.62 and 2d.6, 1.62 and 2d.7, 1.62 and 2d.8, 1.62 and 2d.9, 1.62 and 2d.10, 1.62 and 2d.11, 1.62 and 2d.12, 1.62 and 2d.13, 1.62 and 2d.14, 1.62 and 2d.15, 1.62 and 2d.16, 1.62 and 2d.17, 1.62 and 2d.18, 1.62 and 2d.19, 1.62 and 2d.20, 1.62 and 2d.21, 1.62 and 2d.22, 1.62 and 2d.23, 1.62 and 2d.24, 1.62 and 2d.25, 1.62 and 2d.26, 1.62 and 2d.27, 1.62 and 2d.28,
1.64 and 2d.1, 1.64 and 2d.2, 1.64 and 2d.3, 1.64 and 2d.4, 1.64 and 2d.5, 1.64 and 2d.6, 1.64 and 2d.7, 1.64 and 2d.8, 1.64 and 2d.9, 1.64 and 2d.10, 1.64 and 2d.11, 1.64 and 2d.12, 1.64 and 2d.13, 1.64 and 2d.14, 1.64 and 2d.15, 1.64 and 2d.16, 1.64 and 2d.17, 1.64 and 2d.18, 1.64 and 2d.19, 1.64 and 2d.20, 1.64 and 2d.21, 1.64 and 2d.22, 1.64 and 2d.23, 1.64 and 2d.24, 1.64 and 2d.25, 1.64 and 2d.26, 1.64 and 2d.27, 1.64 and 2d.28,
1.67 and 2d.1, 1.67 and 2d.2, 1.67 and 2d.3, 1.67 and 2d.4, 1.67 and 2d.5, 1.67 and 2d.6, 1.67 and 2d.7, 1.67 and 2d.8, 1.67 and 2d.9, 1.67 and 2d.10, 1.67 and 2d.11, 1.67 and 2d.12, 1.67 and 2d.13, 1.67 and 2d.14, 1.67 and 2d.15, 1.67 and 2d.16, 1.67 and 2d.17, 1.67 and 2d.18, 1.67 and 2d.19, 1.67 and 2d.20, 1.67 and 2d.21, 1.67 and 2d.22, 1.67 and 2d.23, 1.67 and 2d.24, 1.67 and 2d.25, 1.67 and 2d.26, 1.67 and 2d.27, 1.67 and 2d.28,
1.68 and all, 1.68 and 2d.2, 1.68 and 2d.3, 1.68 and 2d.4, 1.68 and 2d.5, 1.68 and 2d.6, 1.68 and 2d.7, 1.68 and 2d.8, 1.68 and 2d.9, 1.68 and 2d.10, 1.68 and 2d.11, 1.68 and 2d.12, 1.68 and 2d.13, 1.68 and 2d.14, 1.68 and 2d.15, 1.68 and 2d.16, 1.68 and 2d.17, 1.68 and 2d.18, 1.68 and 2d.19, 1.68 and 2d.20, 1.68 and 2d.21, 1.68 and 2d.22, 1.68 and 2d.23, 1.68 and 2d.24, 1.68 and 2d.25, 1.68 and 2d.26, 1.68 and 2d.27, 1.68 and 2d.28,
1.69 and 2d.1, 1.69 and 2d.2, 1.69 and 2d.3, 1.69 and 2d.4, 1.69 and 2d.5, 1.69 and 2d.6, 1.69 and 2d.7, 1.69 and 2d.8, 1.69 and 2d.9, 1.69 and 2d.10, 1.69 and 2d.11, 1.69 and 2d.12, 1.69 and 2d.13, 1.69 and 2d.14, 1.69 and 2d.15, 1.69 and 2d.16, 1.69 and 2d.17, 1.69 and 2d.18, 1.69 and 2d.19, 1.69 and 2d.20, 1.69 and 2d.21, 1.69 and 2d.22, 1.69 and 2d.23, 1.69 and 2d.24, 1.69 and 2d.25, 1.69 and 2d.26, 1.69 and 2d.27, 1.69 and 2d.28,
1.70 and 2d.1, 1.70 and 2d.2, 1.70 and 2d.3, 1.70 and 2d.4, 1.70 and 2d.5, 1.70 and 2d.6, 1.70 and 2d.7, 1.70 and 2d.8, 1.70 and 2d.9, 1.70 and 2d.10, 1.70 and 2d.11, 1.70 and 2d.12, 1.70 and 2d.13, 1.70 and 2d.14, 1.70 and 2d.15, 1.70 and to 2d.16, 1.70 and 2d.17, 1.70 and 2d.18, 1.70 and 2d.19, 1.70 and 2d.20, 1.70 and 2d.21, 1.70 and 2d.22, 1.70 and 2d.23, 1.70 and 2d.24, 1.70 and 2d.25, 1.70 and 2d.26, 1.70 and 2d.27, 1.70 and 2d.28,
1.71 and 2d.1, 1.71 and 2d.2, 1.71 and 2d.3, 1.71 and 2d.4, 1.71 and 2d.5, 1.71 and 2d.6, 1.71 and 2d.7, 1.71 and 2d.8, 1.71 and 2d.9, 1.71 and 2d.10, 1.71 and 2d.11, 1.71 and 2d.12, 1.71 and 2d.13, 1.71 and 2d.14, 1.71 and 2d.15, 1.71 and 2d.16, 1.71 and 2d.17, 1.71 and 2d.16, 1.71 and 2d.19, 1.71 and 2d.20, 1.71 and 2d.21, 1.71 and 2d.22, 1.71 and 2d.23, 1.71 and 2d.24, 1.71 and 2d.25, 1.71 and 2d.26, 1.71 and 2d.27, 1.71 and 2d.28,

The proportions in which the active substances 1 and 2d may be used in the active substance combinations according to the invention are variable. Active substances 1 and 2d may possibly be present in the form of their solvates or hydrates. Depending on the choice of the compounds 1 and 2d, the weight ratios which may be used within the scope of the present invention vary on the basis of the different molecular weights of the various compounds and their different potencies.

As a rule, the pharmaceutical combinations according to the invention may contain compounds 1 and 2d in ratios by weight ranging from 100:1 to 1:100, preferably from 50:1 to 1:50, more preferred from 25:1 to 1:25, most preferred 20:1 to 1:20.

For example, without restricting the scope of the invention thereto, preferred combinations may contain 1 and 2d in the following weight ratios:

100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100.

The pharmaceutical compositions according to the invention containing the combinations of 1 and 2d are normally administered so that 1 and 2d are present together in doses of about 100 to 50000 μg, preferably 1000 to 25000 μg, more preferably 1500 to 10000 μg, better still from about 2000 to about 7000 μg, more preferably 2500 to 6000 μg per single dose. For example about 3000 to about 5500 μg of the combination of 1 and 2d according to the invention may be administered once or twice daily to the patient in need thereof. For example, combinations of 1 and 2d according to the invention contain a quantity of 1 and 2d such that the total dosage per single dose is about 100 μg, 150 μg, 200 μg, 250 μg, 300 μg etc. (add stepwise 50 μg) up to 50000 μg, or similar.

The suggested dosages per single dose specified above are not to be regarded as being limited to the numerical values actually stated, but are intended as dosages which are disclosed by way of example. Of course, dosages which may fluctuate about the abovementioned numerical values within a range of about +/−2.5 μg are also included in the values given above by way of example. In these dosage ranges, the active substances 1 and 2d may be present in the weight ratios given above.

For example, without restricting the scope of the invention thereto, the combinations of 1 and 2d according to the invention may contain a quantity of 1 and p38 kinase inhibitor 2d such that, in each individual dose,

100 μg of 1 and 1000 μg of 2d, 100 μg of 1 and 1500 μg of 2d, 100 μg of 1 and 2000 μg of 2d, 100 μg of 1 and 2500 μg of 2d, 100 μg of 1 and 3000 μg of 2d, 100 μg of 1 and 3500 μg of 2d, 100 μg of 1 and 4000 μg of 2d, 100 μg of 1 and 4500 μg of 2d, 100 μg of 1 and 5000 μg of 2d, 100 μg of 1 and 6000 μg of 2d, 100 μg of 1 and 7000 μg of 2d, 100 μg of 1 and 8000 μg of 2d, 100 μg of 1 and 9000 μg of 2d, 100 μg of 1 and 10000 μg of 2d,
200 μg of 1 and 1000 μg of 2d, 200 μg of 1 and 1500 μg of 2d, 200 μg of 1 and 2000 μg of 2d, 200 μg of 1 and 2500 μg of 2d, 200 μg of 1 and 3000 μg of 2d, 200 μg of 1 and 3500 μg of 2d, 200 μg of 1 and 4000 μg of 2d, 200 μg of 1 and 4500 μg of 2d, 200 μg of 1 and 5000 μg of 2d, 200 μg of 1 and 6000 μg of 2d, 200 μg of 1 and 7000 μg of 2d, 200 μg of 1 and 8000 μg of 2d, 200 μg of 1 and 9000 μg of 2d, 200 μg of 1 and 10000 μg of 2d,
500 μg of 1 and 1000 μg of 2d, 500 μg of 1 and 1500 μg of 2d, 500 μg of 1 and 2000 μg of 2d, 500 μg of 1 and 2500 μg of 2d, 500 μg of 1 and 3000 μg of 2d, 500 μg of 1 and 3500 μg of 2d, 500 μg of 1 and 4000 μg of 2d, 500 μg of 1 and 4500 μg of 2d, 500 μg of 1 and 5000 μg of 2d, 500 μg of 1 and 6000 μg of 2d, 500 μg of 1 and 7000 μg of 2d, 500 μg of 1 and 8000 μg of 2d, 500 μg of 1 and 9000 μg of 2d, 500 μg of 1 and 10000 μg of 2d,
1000 μg of 1 and 1000 μg of 2d, 1000 μg of 1 and 1500 μg of 2d, 1000 μg of 1 and 2000 μg of 2d, 1000 μg of 1 and 2500 μg of 2d, 1000 μg of 1 and 3000 μg of 2d, 1000 μg of 1 and 3500 μg of 2d, 1000 μg of 1 and 4000 μg of 2d, 1000 μg of 1 and 4500 μg of 2d, 1000 μg of 1 and 5000 μg of 2d, 1000 μg of 1 and 6000 μg of 2d, 1000 μg of 1 and 7000 μg of 2d, 1000 μg of 1 and 8000 μg of 2d, 1000 μg of 1 and 9000 μg of 2d, 1000 μg of 1 and 10000 μg of 2d, 5000 μg of 1 and 1000 μg of 2d, 5000 μg of 1 and 1500 μg of 2d, 5000 μg of 1 and 2000 μg of 2d, 5000 μg of 1 and 2500 μg of 2d, 5000 μg of 1 and 3000 μg of 2d,
5000 μg of 1 and 3500 μg of 2d, 5000 μg of 1 and 4000 μg of 2d, 5000 μg of 1 and 4500 μg of 2d, 5000 μg of 1 and 5000 μg of 2d, 5000 μg of 1 and 6000 μg of 2d, 5000 μg of 1 and 7000 μg of 2d, 5000 μg of 1 and 8000 μg of 2d, 5000 μg of 1 and 9000 μg of 2d, 5000 μg of 1 and 10000 μg of 2d,
10000 μg of 1 and 1000 μg of 2d, 10000 μg of 1 and 1500 μg of 2d, 10000 μg of 1 and 2000 μg of 2d, 10000 μg of 1 and 2500 μg of 2d, 10000 μg of 1 and 3000 μg of 2d, 10000 μg of 1 and 3500 μg of 2d, 10000 μg of 1 and 4000 μg of 2d, 10000 μg of 1 and 4500 μg of 2d, 10000 μg of 1 and 5000 μg of 2d, 10000 μg of 1 and 6000 μg of 2d, 10000 μg of 1 and 7000 μg of 2d, 10000 μg of 1 and 8000 μg of 2d, 10000 μg of 1 and 9000 μg of 2d, 10000 μg of 1 and 10000 μg of 2d,
25000 μg of 1 and 1000 μg of 2d, 25000 μg of 1 and 1500 μg of 2d, 25000 μg of 1 and 2000 μg of 2d, 25000 μg of 1 and 2500 μg of 2d, 25000 μg of 1 and 3000 μg of 2d, 25000 μg of 1 and 3500 μg of 2d, 25000 μg of 1 and 4000 μg of 2d, 25000 μg of 1 and 4500 μg of 2d, 25000 μg of 1 and 5000 μg of 2d, 25000 μg of 1 and 6000 μg of 2d, 25000 μg of 1 and 7000 μg of 2d, 25000 μg of 1 and 8000 μg of 2d, 25000 μg of 1 and 9000 μg of 2d, 25000 μg of 1 and 10000 μg of 2d,
50000 μg of 1 and 1000 μg of 2d, 50000 μg of 1 and 1500 μg of 2d, 50000 μg of 1 and 2000 μg of 2d, 50000 μg of 1 and 2500 μg of 2d, 50000 μg of 1 and 3000 μg of 2d, 50000 μg of 1 and 3500 μg of 2d, 50000 μg of 1 and 4000 μg of 2d, 50000 μg of 1 and 4500 μg of 2d, 50000 μg of 1 and 5000 μg of 2d, 50000 μg of 1 and 6000 μg of 2d, 50000 μg of 1 and 7000 μg of 2d, 50000 μg of 1 and 8000 μg of 2d, 50000 μg of 1 and 9000 μg of 2d, 50000 μg of 1 and 10000 μg of 2d, are administered.

One embodiment of the invention is a pharmaceutical composition comprising an EGFR kinase inhibitor 1 and an NK₁ antagonist 2e. Binary compositions containing only one active 1 and one active 2e, optionally together with one or more pharmaceutically acceptable excipients or carriers, are preferred. In the pharmaceutical combinations according to the invention preferred NK₁ antagonists 2e is are selected from the group consisting of

fosaprepitant (2e.1), CJ-17493 (2e.2), MK-310 (2e.3), casopitant (2e.4), netupitant (2e.5), SSR-240600 (2e.6), LY-686017 (2e.7), nolpitantium besilate (2e.8), CP-122721 (2e.9), dilopetine (2e.10), GW-597599 (2e.11), cizolirtine (2e.12), vestipitant+paroxetine (2e.13), TA-5538 (2e.14), SLV-317 (2e.15), 823296 (2e.16), SLV-336 (2e.17), Sch-388714 (2e.18), Sch-202451 (2e.19), CP-96345 (2e.20), CP-728663 (2e.21), TKA-457 (2e.22), NKP-608 (2e.23), NIP-530 (2e.24), NiK-004 (2e.25), MPC-4505 (2e.26), substance P-saporin conjugate (2e.27), ATS, SP-PE toxin (2e.28), NIH, PSI-697 (2e.29), UCB-46331 (2e.30), R-116301 (2e.31), KRP-103 (2e.32), SR-48968 derivatives (2e.33), GR-71251 (2e.34), ZD-6021 (2e.35), MEN-11149 (2e.36), L-742694 (2e.37), L-732138 (2e.38) and capsazepine (2e.39),
optionally in the form of enantiomers, mixtures of enantiomers or the racemates.

Even more preferred representatives of component 2e are selected from the group consisting of fosaprepitant (2e.1), CJ-17493 (2e.2), MK-310 (2e.3), casopitant (2e.4), netupitant (2e.5), SSR-240600 (2e.6), LY-686017 (2e.71, nolpitantium besilate (2e.8), CP-122721 (2e.9), dilopetine (2e.10), GW-597599 (2e.11), cizolirtine (2e.12), vestipitant+paroxetine (2e.13), TA-5538 (2e.14), SLV-317 (2e.15) and 823296 (2e.16), optionally in the form of enantiomers, mixtures of enantiomers or the racemates.

Any reference to NK₁ receptor antagonists 2e within the scope of the present invention includes a reference to the salts, preferably pharmacologically acceptable acid addition salts, or derivatives which may be formed from the NK₁ antagonists. Examples of pharmacologically acceptable acid addition salts of the NK₁ antagonists 2e according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Preferred salts are selected from the group consisting of acetate, hydrochloride, hydrobromide, sulphate, phosphate, maleate and methanesulphonate.

The pharmaceutical combinations of 1 and 2e according to the invention are is preferably administered by inhalation. For inhalation suitable inhalable powders may be used which are packed into suitable capsules (inhalettes) and administered using suitable powder inhalers. Alternatively, the drug may be inhaled by the application of suitable inhalation aerosols. These include inhalation aerosols which contain HFA134a, HFA227 or a mixture thereof as propellant gas. The drug may also be inhaled using suitable solutions of the pharmaceutical combination consisting of 1 and 2e.

Especially preferred pharmaceutical compositions according to the invention comprise the following specific combinations of EGFR kinase inhibitors 1 and NK₁ antagonist 2e, either as free bases or pharmacologically acceptable acid addition salts:

1.1 and 2e.1, 1.1 and 2e.2, 1.1 and 2e.3, 1.1 and 2e.4, 1.1 and 2e.5, 1.1 and 2e.6, 1.1 and 2e.7, 1.1 and 2e.8, 1.1 and 2e.9, 1.1 and 2e.10, 1.1 and 2e.11, 1.1 and 2e.12, 1.1 and 2e.13, 1.1 and 2e.14, 1.1 and 2e.15, 1.1 and 2e.16
1.2 and 2e.1, 1.2 and 2e.2, 1.2 and 2e.3, 1.2 and 2e.4, 1.2 and 2e.5, 1.2 and 2e.6, 1.2 and 2e.7, 1.2 and 2e.8, 1.2 and 2e.9, 1.2 and 2e.10, 1.2 and 2e.11, 1.2 and 2e.12, 1.2 and 2e.13, 1.2 and 2e.14, 1.2 and 2e.15, 1.2 and 2e.16
1.8 and 2e.1, 1.8 and 2e.2, 1.8 and 2e.3, 1.8 and 2e.4, 1.8 and 2e.5, 1.8 and 2e.6, 1.8 and 2e.7, 1.8 and 2e.8, 1.8 and 2e.9, 1.8 and 2e.10, 1.8 and 2e.11, 1.8 and 2e.12, 1.8 and 2e.13, 1.8 and 2e.14, 1.8 and 2e.15, 1.8 and 2e.16
1.12 and 2e.1, 1.12 and 2e.2, 1.12 and 2e.3, 1.12 and 2e.4, 1.12 and 2e.5, 1.12 and 2e.6, 1.12 and 2e.7, 1.12 and 2e.8, 1.12 and 2e.9, 1.12 and 2e.10, 1.12 and 2e.11, 1.12 and 2e.12, 1.12 and 2e.13, 1.12 and 2e.14, 1.12 and 2e.15, 1.12 and 2e.16
1.13 and 2e.1, 1.13 and 2e.2, 1.13 and 2e.3, 1.13 and 2e.4, 1.13 and 2e.5, 1.13 and 2e.6, 1.13 and 2e.7, 1.13 and 2e.8, 1.13 and 2e.9, 1.13 and 2e.10, 1.13 and to 2e.11, 1.13 and 2e.12, 1.13 and 2e.13, 1.13 and 2e.14, 1.13 and 2e.15, 1.13 and 2e.16
1.20 and 2e.1, 1.20 and 2e.2, 1.20 and 2e.3, 1.20 and 2e.4, 1.20 and 2e.5, 1.20 and 2e.6, 1.20 and 2e.7, 1.20 and 2e.8, 1.20 and 2e.9, 1.20 and 2e.10, 1.20 and 2e.11, 1.20 and 2e.12, 1.20 and 2e.13, 1.20 and 2e.14, 1.20 and 2e.15, 1.20 and 2e.16,
1.26 and 2e.1, 1.26 and 2e.2, 1.26 and 2e.3, 1.26 and 2e.4, 1.26 and 2e.5, 1.26 and 2e.6, 1.26 and 2e.7, 1.26 and 2e.8, 1.26 and 2e.9, 1.26 and 2e.10, 1.26 and 2e.11, 1.26 and 2e.12, 1.26 and 2e.13, 1.26 and 2e.14, 1.26 and 2e.15, 1.26 and 2e.16,
1.27 and 2e.1, 1.27 and 2e.2, 1.27 and 2e.3, 1.27 and 2e.4, 1.27 and 2e.5, 1.27 and 2e.6, 1.27 and 2e.7, 1.27 and 2e.8, 1.27 and 2e.9, 1.27 and 2e.10, 1.27 and 2e.11, 1.27 and 2e.12, 1.27 and 2e.13, 1.27 and 2e.14, 1.27 and 2e.15, 1.27 and 2e.16
1.28 and 2e.1, 1.28 and 2e.2, 1.28 and 2e.3, 1.28 and 2e.4, 1.28 and 2e.5, 1.28 and 2e.6, 1.28 and 2e.7, 1.28 and 2e.8, 1.28 and 2e.9, 1.28 and 2e.10, 1.28 and 2e.11, 1.28 and 2e.12, 1.28 and 2e.13, 1.28 and 2e.14, 1.28 and 2e.15, 1.28 and 2e.16,
1.33 and 2e.1, 1.33 and 2e.2, 1.33 and 2e.3, 1.33 and 2e.4, 1.33 and 2e.5, 1.33 and 2e.6, 1.33 and 2e.7, 1.33 and 2e.8, 1.33 and 2e.9, 1.33 and 2e.10, 1.33 and 2e.11, 1.33 and 2e.12, 1.33 and 2e.13, 1.33 and 2e.14, 1.33 and 2e.15, 1.33 and 2e.16,
1.45 and 2e.1, 1.45 and 2e.2, 1.45 and 2e.3, 1.45 and 2e.4, 1.45 and 2e.5, 1.45 and 2e.6, 1.45 and 2e.7, 1.45 and 2e.8, 1.45 and 2e.9, 1.45 and 2e.10, 1.45 and 2e.11, 1.45 and 2e.12, 1.45 and 2e.13, 1.45 and 2e.14, 1.45 and 2e.15, 1.45 and 2e.16
1.46 and 2e.1, 1.46 and 2e.2, 1.46 and 2e.3, 1.46 and 2e.4, 1.46 and 2e.5, 1.46 and 2e.6, 1.46 and 2e.7, 1.46 and 2e.8, 1.46 and 2e.9, 1.46 and 2e.10, 1.46 and 2e.11, 1.46 and 2e.12, 1.46 and 2e.13, 1.46 and 2e.14, 1.46 and 2e.15, 1.46 and 2e.16,
1.47 and 2e.1, 1.47 and 2e.2, 1.47 and 2e.3, 1.47 and 2e.4, 1.47 and 2e.5, 1.47 and 2e.6, 1.47 and 2e.7, 1.47 and 2e.8, 1.47 and 2e.9, 1.47 and 2e.10, 1.47 and 2e.11, 1.47 and 2e.12, 1.47 and 2e.13, 1.47 and 2e.14, 1.47 and 2e.15, 1.47 and 2e.16,
1.48 and 2e.1, 1.48 and 2e.2, 1.48 and 2e.3, 1.48 and 2e.4, 1.48 and 2e.5, 1.48 and 2e.6, 1.48 and 2e.7, 1.48 and 2e.8, 1.48 and 2e.9, 1.48 and 2e.10, 1.48 and 2e.11, 1.48 and 2e.12, 1.48 and 2e.13, 1.48 and 2e.14, 1.48 and 2e.15, 1.48 and 2e.16,
1.62 and 2e.1, 1.62 and 2e.2, 1.62 and 2e.3, 1.62 and 2e.4, 1.62 and 2e.5, 1.62 and 2e.6, 1.62 and 2e.7, 1.62 and 2e.8, 1.62 and 2e.9, 1.62 and 2e.10, 1.62 and 2e.11, 1.62 and 2e.12, 1.62 and 2e.13, 1.62 and 2e.14, 1.62 and 2e.15, 1.62 and 2e.16,
1.64 and 2e.1, 1.64 and 2e.2, 1.64 and 2e.3, 1.64 and 2e.4, 1.64 and 2e.5, 1.64 and 2e.6, 1.64 and 2e.7, 1.64 and 2e.8, 1.64 and 2e.9, 1.64 and 2e.10, 1.64 and 2e.11, 1.64 and 2e.12, 1.64 and 2e.13, 1.64 and 2d.14, 1.64 and 2d.15, 1.64 and 2e.16,
1.67 and 2e.1, 1.67 and 2e.2, 1.67 and 2e.3, 1.67 and 2e.4, 1.67 and 2e.5, 1.67 and 2e.6, 1.67 and 2e.7, 1.67 and 2e.8, 1.67 and 2e.9, 1.67 and 2e.10, 1.67 and 2e.11, 1.67 and 2e.12, 1.67 and 2e.13, 1.67 and 2e.14, 1.67 and 2e.15, 1.67 and 2e.16,
1.68 and 2e.1, 1.68 and 2e.2, 1.68 and 2e.3, 1.68 and 2e.4, 1.68 and 2e.5, 1.68 and 2e.6, 1.68 and 2e.7, 1.68 and 2e.8, 1.68 and 2e.9, 1.68 and 2e.10, 1.68 and 2e.11, 1.68 and 2e.12, 1.68 and 2e.13, 1.68 and 2e.14, 1.68 and 2e.15, 1.68 and 2e.16,
1.69 and 2e.1, 1.69 and 2e.2, 1.69 and 2e.3, 1.69 and 2e.4, 1.69 and 2e.5, 1.69 and 2e.6, 1.69 and 2e.7, 1.69 and 2e.8, 1.69 and 2e.9, 1.69 and 2e.10, 1.69 and 2e.11, 1.69 and 2e.12, 1.69 and 2e.13, 1.69 and 2e.14, 1.69 and 2e.15, 1.69 and 2e.16
1.70 and 2e.1, 1.70 and 2e.2, 1.70 and 2e.3, 1.70 and 2e.4, 1.70 and 2e.5, 1.70 and 2e.6, 1.70 and 2e.7, 1.70 and 2e.8, 1.70 and 2e.9, 1.70 and 2e.10, 1.70 and 2e.11, 1.70 and 2e.12, 1.70 and 2e.13, 1.70 and 2e.14, 1.70 and 2e.15, 1.70 and 2e.16,
1.71 and 2e.1, 1.71 and 2e.2, 1.71 and 2e.3, 1.71 and 2e.4, 1.71 and 2e.5, 1.71 and 2e.6, 1.71 and 2e.7, 1.71 and 2e.8, 1.71 and 2e.9, 1.71 and 2e.10, 1.71 and 2e.11, 1.71 and 2e.12, 1.71 and 2e.13, 1.71 and 2e.14, 1.71 and 2e.15, 1.71 and 2e.16.

The proportions in which the active substances 1 and 2e may be used in the active substance combinations according to the invention are variable. Active substances 1 and 2e may possibly be present in the form of their solvates or hydrates. Depending on the choice of the compounds 1 and 2e, the weight ratios which may be used within the scope of the present invention vary on the basis of the different molecular weights of the various compounds and their different potencies.

As a rule, the pharmaceutical combinations according to the invention may contain compounds 1 and 2e in ratios by weight ranging from 100:1 to 1:100, preferably from 50:1 to 1:50, more preferred from 25:1 to 1:25, most preferred 20:1 to 1:20.

For example, without restricting the scope of the invention thereto, preferred combinations may contain 1 and 2e in the following weight ratios:

100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100.

The pharmaceutical compositions according to the invention containing the combinations of 1 and 2e are normally administered so that 1 and 2e are present together in doses of about 100 to 50000 μg, preferably 1000 to 25000 μg, more preferably 1500 to 10000 μg, better still from about 2000 to about 7000 μg, more preferably 2500 to 6000 μg per single dose. For example about 3000 to about 5500 μg of the combination of 1 and 2e according to the invention may be administered once or twice daily to the patient in need thereof. For example, combinations of 1 and 2e according to the invention contain a quantity of 1 and 2e such that the total dosage per single dose is about 100 μg, 150 μg, 200 μg, 250 μg, 300 μg etc. (add stepwise 50 μg) up to 50000 μg, or similar.

The suggested dosages per single dose specified above are not to be regarded as being limited to the numerical values actually stated, but are intended as dosages which are disclosed by way of example. Of course, dosages which may fluctuate about the abovementioned numerical values within a range of about +/−2.5 μg are also included in the values given above by way of example. In these dosage ranges, the active substances 1 and 2e may be present in the weight ratios given above.

For example, without restricting the scope of the invention thereto, the combinations of 1 and 2e according to the invention may contain a quantity of 1 and NK₁ antagonist 2e such that, in each individual dose,

100 μg of 1 and 1000 μg of 2e, 100 μg of 1 and 1500 μg of 2e, 100 μg of 1 and 2000 μg of 2e, 100 μg of 1 and 2500 μg of 2e, 100 μg of 1 and 3000 μg of 2e, 100 μg of 1 and 3500 μg of 2e, 100 μg of 1 and 4000 μg of 2e, 100 μg of 1 and 4500 μg of 2e, 100 μg of 1 and 5000 μg of 2e, 100 μg of 1 and 6000 μg of 2e, 100 μg of 1 and 7000 μg of 2e, 100 μg of 1 and 8000 μg of 2e, 100 μg of 1 and 9000 μg of 2e, 100 μg of 1 and 10000 μg of 2e,
200 μg of 1 and 1000 μg of 2e, 200 μg of 1 and 1500 μg of 2e, 200 μg of 1 and 2000 μg of 2e, 200 μg of 1 and 2500 μg of 2e, 200 μg of 1 and 3000 μg of 2e, 200 μg of 1 and 3500 μg of 2e, 200 μg of 1 and 4000 μg of 2e, 200 μg of 1 and 4500 μg of 2e, 200 μg of 1 and 5000 μg of 2e, 200 μg of 1 and 6000 μg of 2e, 200 μg of 1 and 7000 μg of 2e, 200 μg of 1 and 8000 μg of 2e, 200 μg of 1 and 9000 μg of 2e, 200 μg of 1 and 10000 μg of 2e,
500 μg of 1 and 1000 μg of 2e, 500 μg of 1 and 1500 μg of 2e, 500 μg of 1 and 2000 μg of 2e, 500 μg of 1 and 2500 μg of 2e, 500 μg of 1 and 3000 μg of 2e, 500 μg of 1 and 3500 μg of 2e, 500 μg of 1 and 4000 μg of 2e, 500 μg of 1 and 4500 μg of 2e, 500 μg of 1 and 5000 μg of 2e, 500 μg of 1 and 6000 μg of 2e, 500 μg of 1 and 7000 μg of 2e, 500 μg of 1 and 8000 μg of 2e, 500 μg of 1 and 9000 μg of 2e, 500 μg of 1 and 10000 μg of 2e,
1000 μg of 1 and 1000 μg of 2e, 1000 μg of 1 and 1500 μg of 2e, 1000 μg of 1 and 2000 μg of 2e, 1000 μg of 1 and 2500 μg of 2e, 1000 μg of 1 and 3000 μg of 2e, 1000 μg of 1 and 3500 μg of 2e, 1000 μg of 1 and 4000 μg of 2e, 1000 μg of 1 and 4500 μg of 2e, 1000 μg of 1 and 5000 μg of 2e, 1000 μg of 1 and 6000 μg of 2e, 1000 μg of 1 and 7000 μg of 2e, 1000 μg of 1 and 8000 μg of 2e, 1000 μg of 1 and 9000 μg of 2e, 1000 μg of 1 and 10000 μg of 2e,
5000 μg of 1 and 1000 μg of 2e, 5000 μg of 1 and 1500 μg of 2e, 5000 μg of 1 and 2000 μg of 2e, 5000 μg of 1 and 2500 μg of 2e, 5000 μg of 1 and 3000 μg of 2e, 5000 μg of 1 and 3500 μg of 2e, 5000 μg of 1 and 4000 μg of 2e, 5000 μg of 1 and 4500 μg of 2e, 5000 μg of 1 and 5000 μg of 2e, 5000 μg of 1 and 6000 μg of 2e, 5000 μg of 1 and 7000 μg of 2e, 5000 μg of 1 and 8000 μg of 2e, 5000 μg of 1 and 9000 μg of 2e, 5000 μg of 1 and 10000 μg of 2e,
10000 μg of 1 and 1000 μg of 2e, 10000 μg of 1 and 1500 μg of 2e, 10000 μg of 1 and 2000 μg of 2e, 10000 μg of 1 and 2500 μg of 2e, 10000 μg of 1 and 3000 μg of 2e, 10000 μg of 1 and 3500 μg of 2e, 10000 μg of 1 and 4000 μg of 2e, 10000 μg of 1 and 4500 μg of 2e, 10000 μg of 1 and 5000 μg of 2e, 10000 μg of 1 and 6000 μg of 2e, 10000 μg of 1 and 7000 μg of 2e, 10000 μg of 1 and 8000 μg of 2e, 10000 μg of 1 and 9000 μg of 2e, 10000 μg of 1 and 10000 μg of 2e,
25000 μg of 1 and 1000 μg of 2e, 25000 μg of 1 and 1500 μg of 2e, 25000 μg of 1 and 2000 μg of 2e, 25000 μg of 1 and 2500 μg of 2e, 25000 μg of 1 and 3000 μg of 2e, 25000 μg of 1 and 3500 μg of 2e, 25000 μg of 1 and 4000 μg of 2e, 25000 μg of 1 and 4500 μg of 2e, 25000 μg of 1 and 5000 μg of 2e, 25000 μg of 1 and 6000 μg of 2e, 25000 μg of 1 and 7000 μg of 2e, 25000 μg of 1 and 8000 μg of 2e, 25000 μg of 1 and 9000 μg of 2e, 25000 μg of 1 and 10000 μg of 2e,
50000 μg of 1 and 1000 μg of 2e, 50000 μg of 1 and 1500 μg of 2e, 50000 μg of 1 and 2000 μg of 2e, 50000 μg of 1 and 2500 μg of 2e, 50000 μg of 1 and 3000 μg of 2e, 50000 μg of 1 and 3500 μg of 2e, 50000 μg of 1 and 4000 μg of 2e, 50000 μg of 1 and 4500 μg of 2e, 50000 μg of 1 and 5000 μg of 2e, 50000 μg of 1 and 6000 μg of 2e, 50000 μg of 1 and 7000 μg of 2e, 50000 μg of 1 and 8000 μg of 2e, 50000 μg of 1 and 9000 μg of 2e, 50000 μg of 1 and 10000 μg of 2e, are administered.

One embodiment of the invention is a pharmaceutical composition comprising an EGFR kinase inhibitor 1 and an anticholinergic 2f. Binary compositions containing only one active 1 and one active 2e, optionally together with one or more pharmaceutically acceptable excipients or carriers, are preferred. In the pharmaceutical combinations according to the invention preferred anticholinergic 2e are selected from the group consisting of

Tiotropium salts 2f.1, preferred the bromide salt, Oxitropium salts 2f.2, preferred the bromide salt, Flutropium salts 2f.3, preferred the bromide salt, Ipratropium salts 2f.4, preferred the bromide salt, Glycopyrronium salts 2f.5, preferred the bromide salt, Trospium salts 2f.6 preferred the chloride salt, and Tolterodin 2f.7 or the anticholinergic 2f is selected from the group consisting of
2,2-Diphenylpropion acid tropenolester-methobromide 2f.8,
2,2-Diphenylpropion acid scopinester-methobromide 2f.9,
2-Fluor-2,2-Diphenylacetic acid scopinester-methobromide 2f.10
2-Fluor-2,2-Diphenylacetic acid tropenolester-methobromide 2f.11,
3,3′,4,4′-Tetrafluorbenzil acid tropenolester-Methobromide 2f.12,
3,3′,4,4′-Tetrafluorbenzil acid scopinester-Methobromide 2f.13,
4,4′-Difluorbenzil acid tropenolester-Methobromide 2f.14,
4,4′-Difluorbenzil acid scopinester-Methobromide 2f.15,
3,3′-Difluorbenzil acid tropenolester-Methobromide 2f.16,
3,3′-Difluorbenzil acid scopinester-Methobromide 2f.17,
9-Hydroxy-fluoren-9-carbon acid tropenolester-Methobromide2f.18
9-Fluor-fluoren-9-carbon acid tropenolester-Methobromide 2f.19,
9-Hydroxy-fluoren-9-carbon acid scopinester-Methobromide2f.20
9-Fluor-fluoren-9-carbon acid scopinester Methobromide 2f.21,
9-Methyl-fluoren-9-carbon acid tropenolesterMethobromide 2f.22,
9-Methyl-fluoren-9-carbon acid scopinesterMethobromide 2f.23,
Benzil acid cyclopropyltropinester-Methobromide 2f.24,
2,2-Diphenylpropion acid cyclopropyltropinester-Methobromide 2f.25,
9-Hydroxy-xanthen-9-carbon acid cyclopropyltropinesterMethobromide 2f.26,
9-Methyl-fluoren-9-carbon acid cyclopropyltropinester-Methobromide 2f.27,
9-Methyl-xanthen-9-carbon acid cyclopropyltropinester-Methobromide 2f.28,
9-Hydroxy-fluoren-9-carbon acid cyclopropyltropinester-Methobromide 21.29,
4,4′-Difluorbenzil acid methylestercyclopropyltropinester-Methobromide 2f.30
9-Hydroxy-xanthen-9-carbon acid tropenolester-Methobromide 2f.31,
9-Hydroxy-xanthen-9-carbon acid scopinester Methobromide 21.32,
9-Methyl-xanthen-9-carbon acid tropenolester-Methobromide 2f.33,
9-Methyl-xanthen-9-carbon acid scopinesterMethobromide 21.34,
9-Ethyl-xanthen-9-carbon acid tropenolester Methobromide 2f.35,
9-Difluormethyl-xanthen-9-carbon acid tropenolester-Methobromide 21.36 and
9-Hydroxymethyl-xanthen-9-carbon acid scopinester-Methobromide 2f.37.
   and the pharmacologically acceptable salts thereof or
   the anticholinergic 2f is selected from the group consisting of the compounds of formula 2f.I

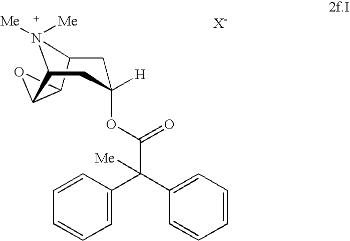

wherein
X⁻ denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate, preferably chloride, bromide, p-toluenesulphonate and methanesulphonate, particularly preferred bromide.

The salts of formula 2f.I are known from International Patent Application WO 02/32899.

The pharmaceutical compositions according to the invention containing the combinations of 1 and 2f are normally used so that 1 and 2f may be present together in doses from 1000 to 100,000 μg, preferably from 1500 to 50,000 μg, more preferably from 2000 to 10,000 μg, even more preferably from 2500 to 7500 μg per single dose. For example, combinations of 1 and 2f according to the invention contain an amount of 1 and 2f such that the total dosage per single dose is 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, 7000 μg, 7050 μg, 7100 μg, 7150 μg, 7200 μg, 7250 μg, 7300 μg, 7350 μg, 7400 μg, 7450 μg, 7500 μg or the like. These proposed dosages per single dose are not to be regarded as being restricted to the numerical values explicitly mentioned but are merely disclosed by way of example. Obviously, dosages which fluctuate around these values within a range of about +/−25 μg are also covered by the values mentioned by way of example. In these dosage ranges the active substances 1 and 2f may be present in the weight ratios described below.

For example and without restricting the scope of the invention thereto, the combinations of 1 and 2 according to the invention may contain an amount of 1 and 2f such that 16.5 μg of 2f and 2500 μg of 1, 16.5 μg of 2f and 3000 μg of 1, 16.5 μg of 2f and 3500 μg of 1, 16.5 μg of 2f and 4000 μg of 1, 16.5 μg of 2f and 4500 μg of 1, 16.5 μg of 2f and 5000 μg of 1, 16.5 μg of 2f and 5500 μg of 1, 16.5 μg of 2f and 6000 μg of 1, 16.5 μg of 2f and 6500 μg of 1, 16.5 μg of 2f and 7000 μg of 1, 33.1 μg of 2f and 2500 μg of 1, 33.1 μg of 2f and 3000 μg of 1, 33.1 μg of 2f and 3500 μg of 133.1 μg of 2f and 4000 μg of 1, 33.1 μg of 2f and 4500 μg of 1, 33.1 μg of 2f and 5000 μg of 1, 33.1 μg of 2f and 5500 μg of 1, 33.1 μg of 2f and 6000 μg of 1, 33.1 μg of 2f and 6500 μg of 1, 33.1 μg of 2f and 7000 μg of 1, 49.5 μg of 2f and 2500 μg of 1, 49.5 μg of 2f and 3000 μg of 1, 49.5 μg of 2f and 3500 μg of 1, 49.5 μg of 2f and 4000 μg of 1, 49.5 μg of 2f and 4500 μg of 1, 49.5 μg of 21 and 5000 μg of 1, 49.5 μg of 2f and 5500 μg of 1, 49.5 μg of 2f and 6000 μg of 1, 49.5 μg of 2f and 6500 μg of 1, 49.5 μg of 2f and 7000 μg of 1, 82.6 μg of 2f and 2500 μg of 1, 82.6 μg of 2f and 3000 μg of 1, 82.6 μg of 21 and 3500 μg of 1, 82.6 μg of 2f and 4000 μg of 1, 82.6 μg of 2f and 4500 μg of 1, 82.6 μg of 2f and 5000 μg of 1, 82.6 μg of 2f and 5500 μg of 1, 82.6 μg of 21 and 6000 μg of 1, 82.6 μg of 2f and 6500 μg of 1, 82.6 μg of 2f and 7000 μg of 1, 165.1 μg of 2f and 2500 μg of 1, 165.1 μg of 2f and 3000 μg of 1, 165.1 μg of 21 and 3500 μg of 1, 165.1 μg of 2f and 4000 μg of 1, 165.1 μg of 21 and 4500 μg of 1, 165.1 μg of 2f and 5000 μg of 1, 165.1 μg of 2f and 5500 μg of 1, 165.1 μg of 2f and 6000 μg of 165.1 μg of 2f and 6500 μg of 1, 165.1 μg of 2f and 7000 μg of 1, 206.4 μg of 2f and 2500 μg of 1, 206.4 μg of 2f and 3000 μg of 1, 206.4 μg of 2f and 3500 μg of 1, 206.4 μg of 2f and 4000 μg of 1, 206.4 μg of 2f and 4500 μg of 1, 206.4 μg of 2f and 5000 μg of 1, 206.4 μg of 2f and 5500 μg of 1, 206.4 μg of 2f and 6000 μg of 1, 206.4 μg of 2f and 6500 μg of 1, 206.4 μg of 2f and 7000 μg of 1, 412.8 μg of 2f and 2500 μg of 1, 412.8 μg of 2f and 3000 μg of 1, 412.8 μg of 2f and 3500 μg of 1, 412.8 μg of 2f and 4000 μg of 1, 412.8 μg of 21 and 4500 μg of 1, 412.8 μg of 2f and 5000 μg of 1, 412.8 μg of 2f and 5500 μg of 1, 412.8 μg of 2f and 6000 μg of 1, 412.8 μg of 2f and 6500 μg of 1 or 412.8 μg of 2f and 7000 μg of 1 are administered per single dose.

If the active substance combination wherein 2f denotes the bromide is used as the preferred combination of 1 and 2f according to the invention, the quantities of active substances 1 and 2f administered per single dose as specified by way of example correspond to the following quantities of 1 and 2f administered

per single dose: 20 μg of 2f and 2500 μg of 1, 20 μg of 2f and 3000 μg of 1, 20 μg of 2f and 3500 μg of 1, 20 μg of 2f and 4000 μg of 1, 20 μg of 2f and 4500 μg of 1, 20 μg of 2f and 5000 μg of 1, 20 μg of 2f and 5500 μg of 1, 20 μg of 2f and 6000 μg of 1, 20 μg of 2f and 6500 μg of 1, 20 μg of 2f and 7000 μg of 1, 40 μg of 21 and 2500 μg of 1, 40 μg of 2f and 3000 μg of 1, 40 μg of 2f and 3500 μg of 1, 40 μg of 2f and 4000 μg of 1, 40 μg of 2f and 4500 μg of 1, 40 μg of 2f and 5000 μg of 1, 40 μg of 2f and 5500 μg of 1, 40 μg of 2f and 6000 μg of 1, 40 μg of 2f and 6500 μg of 1.40 μg of 2f and 7000 μg of 1, 60 μg of 2f and 2500 μg of 1, 60 μg of 2f and 3000 μg of 1.60 μg of 2f and 3500 μg of 1, 60 μg of 2f and 4000 μg of 1, 60 μg of 2f and 4500 μg of 1, 60 μg of 2f and 5000 μg of 2f, 60 μg of 2f and 5500 μg of 21, 60 μg of 2f and 6000 μg of 2f, 60 μg of 2f and 6500 μg of 21, 60 μg of 2f and 7000 μg of 2f, 100 μg of 2f and 2500 μg of 2f, 100 μg of 2f and 3000 μg of 2f, 100 μg of 2f and 3500 μg of 2f, 100 μg of 2f and 4000 μg of 2f, 100 μg of 2f and 4500 μg of 2f, 100 μg of 2f and 5000 μg of 2f, 100 μg of 2f and 5500 μg of 2f, 100 μg of 2f and 6000 μg of 2f, 100 μg of 2f and 6500 μg of 2f, 100 μg of 2f and 7000 μg of 2f, 200 μg of 2f and 2500 μg of 2f, 200 μg of 2f and 3000 μg of 2f, 200 μg of 2f and 3500 μg of 2f, 200 μg of 2f and 4000 μg of 2f, 200 μg of 2f and 4500 μg of 2f, 200 μg of 2f and 5000 μg of 2f, 200 μg of 2f and 5500 μg of 2f, 200 μg of 2f and 6000 μg of 2f, 200 μg of 2f and 6500 μg of 2f, 200 μg of 21 and 7000 μg of 2f, 250 μg of 2f and 2500 μg of 2f, 250 μg of 2f and 3000 μg of 2f, 250 μg of 2f and 3500 μg of 2f, 250 μg of 2f and 4000 μg of 2f, 250 μg of 2f and 4500 μg of 2f, 250 μg of 2f and 5000 μg of 2f, 250 μg of 2f and 5500 μg of 2f, 250 μg of 2f and 6000 μg of 2f, 250 μg of 2f and 6500 μg of 2f or 250 μg of 2f and 7000 μg of 2f, 500 μg of 2f and 2500 μg of 2f, 500 μg of 2f and 3000 μg of 2f, 500 μg of 2f and 3500 μg of 2f, 500 μg of 2f and 4000 μg of 2f, 500 μg of 21 and 4500 μg of 2f, 500 μg of 2f and 5000 μg of 2f, 500 μg of 2f and 5500 μg of 2f, 500 μg of 2f and 6000 μg of 2f, 500 μg of 2f and 6500 μg of 2f or 500 μg of 2f and 7000 μg off.

The active substance combinations of 1 and 2f according to the invention are preferably administered by inhalation. For this purpose, ingredients 1 and 2f have to be made available in forms suitable for inhalation. Inhalable preparations include inhalable powders, propellant-containing metering aerosols or propellant-free inhalable solutions. Inhalable powders according to the invention containing the combination of active substances 1 and 2f may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2f either together in one formulation or in two or three separate formulations.

One embodiment of the invention is a pharmaceutical composition comprising an EGFR kinase inhibitor 1 and an endothelin-antagonist 2g. Binary compositions containing only one active 1 and one active 2g, optionally together with one or more pharmaceutically acceptable excipients or carriers, are preferred, In the pharmaceutical combinations according to the invention preferred endothelin-antagonists 2g are selected from the group consisting of ambrisentan 2g.1, sitaxsentan 2g.2 and TBC 3711 2g.3, and the pharmacologically acceptable salts thereof.

Any reference to endothelin-antagonists 2g within the scope of the present invention includes a reference to the salts, preferably pharmacologically acceptable acid addition salts, or derivatives which may be formed from the endothelin-antagonists. Examples of pharmacologically acceptable acid addition salts of the endothelin-antagonists 2g according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Preferred salts are selected from the group consisting of acetate, hydrochloride, hydrobromide, sulphate, phosphate, maleate and methanesulphonate.

Any reference to the abovementioned endothelin-antagonists 2g within the scope of the present invention includes a reference to any alkali metal and alkaline earth metal to salts thereof which may exist, If the compounds 2g, are present in the form of their basic salts, the sodium or potassium salts are particularly preferred.

The pharmaceutical combinations of 1 and 2g according to the invention are preferably administered by parenteral or oral route or by inhalation, the latter being particularly preferred. For oral or parenteral administration the pharmaceutical compositions according to the invention may be administered e.g. in the form of solutions and tablets. For inhalation, as preferred according to the invention, suitable inhalable powders may be used which are packed into suitable capsules (inhalettes) and administered using suitable powder inhalers. Alternatively, the drug may be inhaled by the application of suitable inhalation aerosols. These include inhalation aerosols which contain HFA134a, HFA227 or a mixture thereof as propellant gas. The drug may also be inhaled using suitable solutions of the pharmaceutical combination consisting of 1 and 2g.

Especially preferred pharmaceutical compositions according to the invention comprise the following specific combinations of EGFR kinase inhibitors 1 and endothelin-antagonists 2g, either as free bases or pharmacologically acceptable acid addition salts:

1.1 and 2g.1, 1.4 and 2g.1, 1.6 and 2g.1, 1.8 and 2g.1, 1.9 and 2g.1, 1.14 and 2g.1, 1.17 and 2g.1, 1.19 and 2g.1, 1.21 and 2g.1, 1.23 and 2g.1, 1.24 and 2g.1, 1.27 and 2g.1, 1.28 and 2g.1, 1.30 and 2g.1, 1.34 and 2g.1, 1.35 and 2g.1, 1.37 and 2g.1, 1.38 and 2g.1, 1.40 and 2g.1, 1.42 and 2g.1, 1.43 and 2g.1, 1.44 and 2g.1, 1.48 and 2g.1, 1.52 and 2g.1, 1.55 and 2g.1, 1.57 and 2g.1, 1.59 and 2g.1, 1.60 and 2g.1, 1.63 and 2g.1, 1.64 and 2g.1, 1.66 and 2g.1, 1.67 and 2g.1, 1.69 and 2g.1, 1.70 and 2g.1, 1.71 and 2g.1, 1.72 and 2g.1, 1.78 and 2g.1, 1.82 and 2g.1, 1.83 and 2g.1, 1.84 and 2g.1, 1.88 and 2g.1, 1.90 and 2g.1, 1.91 and 2g.1, 1.94 and 2g.1, 1.95 and 2g.1;
1.1 and 2g.2, 1.4 and 2g.2, 1.6 and 2g.2, 1.8 and 2g2, 1.9 and 2g.2, 1.14 and 2g.2, 1.17 and 2g.2, 1.19 and 2g.2, 1.21 and 2g.1, 1.23 and 2g.2, 1.24 and 2g.2, 1.27 and 2g.2, 1.28 and 2g.2, 1.30 and 2g.2, 1.34 and 2g.2, 1.35 and 2g.2, 1.37 to and 2g.2, 1.38 and 2g.2, 1.40 and 2g.2, 1.42 and 2g.2, 1.43 and gig, 1.44 and 2g.2, 1.48 and 2g.2, 1.52 and 2g.2, 1.55 and 2g.2, 1.57 and 2g.2, 1.59 and 2g.2, 1.60 and 2g.2, 1.63 and gig, 1.64 and 2g.2, 1.66 and 2g.1, 1.67 and 2g.2, 1.69 and 2g.2, 1.70 and 2g.2, 1.71 and 2g.2, 1.72 and 2g.2, 1.78 and 2g.2, 1.82 and 2g.2, 1.83 and 2g.2, 1.84 and 2g.2, 1.88 and 2g.2, 1.90 and 2g.2, 1.91 and 2g.2, 1.94 and 2g.2, 1.95 and 2g.2;
1.1 and 2g.3, 1.4 and 2g.2, 1.6 and 2g.3, 1.8 and 2g.2, 1.9 and 2g.3, 1.14 and 2g.3, 1.17 and 2g.3, 1.19 and 2g.2, 1.21 and 2g.3, 1.23 and 2g.3, 1.24 and 2g.3, 1.27 and 2g.3, 1.28 and 2g.3, 1.30 and 2g.3, 1.34 and 2g.3, 1.35 and 2g.3, 1.37 and 2g.2, 1.38 and 2g.2, 1.40 and 2g.2, 1.42 and 2g.2, 1.43 and 2g.3, 1.44 and 2g.3, 1.48 and 2g.3, 1.52 and 2g.3, 1.55 and 2g.3, 1.57 and 2g.3, 1.59 and 2g.3, 1.60 and 2g.2, 1.63 and 2g.2, 1.64 and 2g.2, 1.66 and 2g.2, 1.67 and 2g.2, 1.69 and 2g.3, 1.70 and 2g.3, 1.71 and 2g.3, 1.72 and 2g.3, 1.78 and 2g.3, 1.82 and 2g.3, 1.83 and 221, 1.84 and 2g.3, 1.88 and 2g.3, 1.90 and 2g.3, 1.91 and 2g.3, 1.94 and 2g.3, 1.95 and 2g.3;

The proportions in which the active substances 1 and 2g, may be used in the active substance combinations according to the invention are variable. Active substances 1 and 2g may possibly be present in the form of their solvates or hydrates. Depending on the choice of the compounds 1 and 2g, the weight ratios which may be used within the scope of the present invention vary on the basis of the different molecular weights of the various salt forms.

As a rule, the pharmaceutical combinations according to the invention may contain compounds 1 and 2g in ratios by weight ranging from 100:1 to 1:100, preferably from 50:1 to 1:50, more preferred from 25:1 to 1:25, most preferred 20:1 to 1:20.

For example, without restricting the scope of the invention thereto, preferred combinations may contain 1 and an endothelin-antagonists 2g in the following weight ratios:

100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100.

The pharmaceutical compositions according to the invention containing the combinations of 1 and 2g are normally administered so that 1 and 2g are present together in doses of about 100 to 50000 μg, preferably 1000 to 25000 μg, more preferably 1500 to 10000 μg, better still from about 2000 to about 7000 μg, more preferably 2500 to 6000 μg per single dose. For example about 3000 to about 5500 μg of the combination of 1 and 2g according to the invention may be administered once or twice daily to the patient in need thereof. For example, combinations of 1 and 2g according to the invention contain a quantity of 1 and an endothelin-antagonist 2g (as for instance 2g.1, 2g.2 or 2g.3 such that the total dosage per single dose is about 100 μg, 150 μg, 200 μg, 250 μg, 300 μg etc. (add stepwise 50 μg) up to 50000 μg, or similar.

For example, without restricting the scope of the invention thereto, the combinations of 1 and 2g according to the invention may contain a quantity of 1 and an endothelin-antagonist 2g (as for instance 2g.1, 2g.2 or 2g.3) in such an amount that the following quantities of the active substances are administered per single dose: 100 μg of 1 and 1000 μg of 2g, 100 μg of 1 and 1500 μg of as 100 μg of 1 and 2000 μg of 2g, 100 μg of 1 and 2500 μg of 2g, 100 μg of 1 and 3000 μg of 2g, 100 μg of 1 and 3500 μg of 2g, 100 μg of 1 and 4000 μg of 2g, 100 μg of 1 and 4500 μg of 2g, 100 μg of 1 and 5000 μg of 2g, 100 μg of 1 and 6000 μg of 2g, 100 μl of 1 and 7000 μg of 2g, 100 μg of 1 and 8000 μg of 2g, 100 μg of 1 and 9000 μg of 2g, 100 μg of 1 and 10000 μg of 2g,

200 μg of 1 and 1000 μg of 2g, 200 μg of 1 and 1500 μg of 2g, 200 μg of 1 and 2000 μg of 2g, 200 μg of 1 and 2500 μg of 2g, 200 μg of 1 and 3000 μg of 2g, 200 μg of 1 and 3500 μg of 2g, 200 μg of 1 and 4000 μg of 22, 200 μg of 1 and 4500 μg of 2g, 200 μg of 1 and 5000 μg of 2g, 200 μg of 1 and 6000 μg of 2g, 200 μg of 1 and 7000 μg of 2g, 200 μg of 1 and 8000 μg of as 200 μg of 1 and 9000 μg of aa, 200 μg of 1 and 10000 μg of 2g,
500 μg of 1 and 1000 μg of 2g, 500 μg of 1 and 1500 μg of 2g, 500 μg of 1 and 2000 μg of 2g, 500 μg of 1 and 2500 μg of 2g, 500 μg of 1 and 3000 μg of 2g, 500 μg of 1 and 3500 μg of 2g, 500 μg of 1 and 4000 μg of 2g, 500 μg of 1 and 4500 μg of 2g, 500 μg of 1 and 5000 μg of 2g, 500 μg of 1 and 6000 μg of 2g, 500 μg of 1 and 7000 μg of 2g, 500 μg of 1 and 8000 μg of as 500 μg of 1 and 9000 μg of 2g, 500 μg of 1 and 10000 μg of 2g,
1000 μg of 1 and 1000 μg of as 1000 μg of 1 and 1500 μg of 2g, 1000 μg of 1 and 2000 μg of 2g, 1000 μg of 1 and 2500 μg of 2g, 1000 μg of 1 and 3000 μg of 2g, 1000 μg of 1 and 3500 μg of 2a, 1000 μg of 1 and 4000 μg of 2g, 1000 μg of 1 and 4500 μg of 2g, 1000 μg of 1 and 5000 μg of 2g, 1000 μg of 1 and 6000 μg of 2g, 1000 μg of 1 and 7000 μg of 2g, 1000 μg of 1 and 8000 μg of aa, 1000 μg of 1 and 9000 μg of as 1000 μg of 1 and 10000 μg of 2g,
5000 μg of 1 and 1000 μg of 2g, 5000 μg of 1 and 1500 μg of 2g, 5000 μg of 1 and 2000 μg of 2g, 5000 μg of 1 and 2500 μg of 2g, 5000 μg of 1 and 3000 μg of 2g, 5000 μg of 1 and 3500 μg of as 5000 μg of 1 and 4000 μg of 2g, 5000 μg of 1 and 4500 μg of 2g, 5000 μg of 1 and 5000 μg of 2g, 5000 μg of 1 and 6000 μg of 2g, 5000 μg of 1 and 7000 μg of 2g, 5000 μg of 1 and 8000 μg of 2g, 5000 μg of 1 and 9000 μg of 2g, 5000 μg of 1 and 10000 μg of 2g,
10000 μg of 1 and 1000 μg of 2g, 10000 μg of 1 and 1500 μg of as 10000 μg of 1 and 2000 μg of as 10000 μg of 1 and 2500 μg of as 10000 μg of 1 and 3000 μg of 2g, 10000 μg of 1 and 3500 μg of as 10000 μg of 1 and 4000 μg of as 10000 μg of 1 and 4500 μg of as 10000 μg of 1 and 5000 μg of 2g, 10000 μg of 1 and 6000 μg of as 10000 μg of 1 and 7000 μg of as 10000 μg of 1 and 8000 μg of as 10000 μg of 1 and 9000 μg of as 10000 μg of 1 and 10000 μg of 2g,
25000 μg of 1 and 1000 μg of 2g, 25000 μg of 1 and 1500 μg of 2g, 25000 μg of 1 and 2000 μg of 2g, 25000 μg of 1 and 2500 μg of 2g, 25000 μg of 1 and 3000 μg of 2g, 25000 μg of 1 and 3500 μg of as 25000 μg of 1 and 4000 μg of 2g, 25000 μg of 1 and 4500 μg of 2g, 25000 μg of 1 and 5000 μg of 2g, 25000 μg of 1 and 6000 μg of 2g, 25000 μg of 1 and 7000 μg of 2g, 25000 μg of 1 and 8000 μg of 2g, 25000 μg of 1 and 9000 μg of 2g, 25000 μg of 1 and 10000 μg of 2g,
50000 μg of 1 and 1000 μg of 2g, 50000 μg of 1 and 1500 μg of as 50000 μg of 1 and 2000 μg of 2g, 50000 μg of 1 and 2500 μg of 2g, 50000 μg of 1 and 3000 μg of 2g, 50000 μg of 1 and 3500 μg of 2g, 50000 μg of 1 and 4000 μg of 2g, 50000 μg of 1 and 4500 μg of al 50000 μg of 1 and 5000 μg of as 50000 μg of 1 and 6000 μg of 2g, 50000 μg of 1 and 7000 μg of 2g, 50000 μg of 1 and 8000 μg of 2g, 50000 μg of 1 and 9000 μg of 2g, 50000 μg of 1 and 10000 μg of 2g, are administered.

The suggested dosages per single dose specified above are not to be regarded as being limited to the numerical values actually stated, but are intended as dosages which are disclosed by way of example. Of course, dosages which may fluctuate about the abovementioned numerical values within a range of about +/−2.5 μg are also included in the values given above by way of example. In these dosage ranges, the active substances 1 and 2g may be present in the weight ratios given above.

In the pharmaceutical combinations according to the invention a preferred EGFR kinase inhibitor 1 is selected from the group consisting of
(1.1) 4-[(3-chloro-4-fluoro-phenyl)amino]-6-[2-((S)-6-methyl-2-oxo-morpholin-4-yl)-ethoxy]-7-methoxy-quinazoline,
(1.2) 4-[(3-chloro-4-fluoro-phenyl)amino]-6-[2-(2,2-dimethyl-6-oxo-morpholin-4-yl)-ethoxy]-7-methoxy-quinazoline,
(1.8) 4-[(3-chloro-4-fluoro-phenyl)amino]-6-(tetrahydropyran-3-yloxy)-7-methoxy-quinazoline,
(1.12) 4-[(3-chloro-4-fluoro-phenyl)amino]-6-(piperidin-3-yloxy)-7-methoxy-quinazoline,
(1.13) 4-[(3-chloro-4-fluoro-phenyl)amino]-6-[1-(2-acetylamino-ethyl)-piperidin-4-yl-oxy]-7-methoxy-quinazoline,
(1.25) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.26) 4-[(3-chloro-4-fluoro-phenyl)amino]-6-(cis-4-{N-[(morpholin-4-yl)sulfonyl]-N-methyl-amino}-cyclohexan-1-yloxy)-7-methoxy-quinazoline,
(1.27) 4-[(3-chloro-4-fluoro-phenyl)amino]-6-(trans-4-ethansulfonylamino-cyclohexan-1-yloxy)-7-methoxy-quinazoline,
(1.28) 4-[(3-chloro-4-fluoro-phenyl)amino]-6-(1-methansulfonyl-piperidin-4-yloxy)-7-ethoxy-quinazoline,
(1.33) 4-[(3-ethinyl-phenyl)amino]-6-(tetrahydropyran-4-yloxy]-7-methoxy-quinazoline,
(1.45) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.46) 4-[(3-ethinyl-phenyl)amino]-6-(piperidin-4-yloxy)-7-methoxy-quinazoline,
(1.47) 4-[(3-ethinyl-phenyl)amino]-6-[1-(2-methoxy-acetyl)-piperidin-4-yloxy]-7-methoxy-quinazoline,
(1.48) 4-[(3-ethinyl-phenyl)amino]-6-{1-[(morpholin-4-yl)carbonyl]-piperidin-4-yloxy}-7-methoxy-quinazoline,
(1.62) 4-[(3-chloro-4-fluoro-phenyl)amino]-6-{1-[2-(2-oxo-3-methyl-imidazoildin-1-yl)-ethyl]-piperidin-4-yloxy}-7-methoxy-quinazoline,
(1.64) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.67) 4-[(3-chloro-4-fluoro-phenyl)amino]-6-(tetrahydropyran-4-yloxy)-7-methoxy-quinazoline,
(1.68) 4-[(3-chloro-4-fluoro-phenyl)amino]-6-(1-methylcarbonyl-piperidin-4-yloxy)-7-methoxy-quinazoline,
(1.69) 4-[(3-chloro-4-fluoro-phenyl)amino]-6-(1-dimethylaminoacetyl-piperidin-4-yloxy)-7-methoxy-quinazoline,
(1.70) 4-[(3-chloro-4-fluoro-phenyl)amino]-8-{1-[(dimethylamino)carbonylmethyl]-piperidin-4-yloxy}-7-methoxy-quinazoline and
(1.71) 4-[(3-chloro-4-fluoro-phenyl)amino]-6-(1-methansulfonyl-pipendin-4-yloxy)-quinazoline,

Particularly preferred is an EGFR kinase inhibitor 1 selected from the group consisting of
(1.1) 4-[(3-chloro-4-fluoro-phenyl)amino]-6-[2-((S)-6-methyl-2-oxo-morpholin-4-yl)-ethoxy]-7-methoxy-quinazoline
(1.2) 4-[(3-chloro-4-fluoro-phenyl)amino]-6-[2-(2,2-dimethyl-6-oxo-morpholin-4-yl)-ethoxy]-7-methoxy-quinazoline,
(1.13) 4-[(3-chloro-4-fluoro-phenyl)amino]-6-[1-(2-acetylamino-ethyl)-piperidin-4-yl-oxy]-7-methoxy-quinazoline,
(1.25) 4-[(3-chloro-4-fluoro-phenyl)amino]-6-(cis-4-{N-[(morpholin-4-yl)carbonyl]N-methyl-amino}-cyclohexan-1-yloxy)-7-methoxy-quinazoline,
(1.26) 4-[(3-chloro-4-fluoro-phenyl)amino]-6-(cis-4-{N-[(morpholin-4-yl)sulfonyl]-N-methyl-amino}-cyclohexan-1-yloxy)-7-methoxy-quinazoline,
(1.27) 4-[(3-chloro-4-fluoro-phenyl)amino]-6-(trans-4-ethansulfonylamino-cyclohexan-1-yloxy)-7-methoxy-quinazoline,
(1.28) 4-[(3-chloro-4-fluoro-phenyl)amino]-6-(1-methansulfonyl-piperidin-4-yloxy)-7-ethoxy-quinazoline,
(1.33) 4-[(3-ethinyl-phenyl)amino]-6-(tetrahydropyran-4-yloxy]-7-methoxy-quinazoline,
(1.45) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.46) 4-[(3-ethinyl-phenyl)amino]-6-(piperidin-4-yloxy)-7-methoxy-quinazoline,
(1.47) 4-[(3-ethinyl-phenyl)amino]-6-[1-(2-methoxy-acetyl)-piperidin-4-yloxy]-7-methoxy-quinazoline,
(1.48) 4-[(3-ethinyl-phenyl)amino]-6-{1-[(morpholin-4-yl)carbonyl]-piperidin-4-yloxy}-7-methoxy-quinazoline,
(1.62) 4-[(3-chloro-4-fluoro-phenyl)amino]-6-{1-[2-(2-oxo-3-methyl-imidazolidin-1-yl)-ethyl]-piperidin-4-yloxy}-7-methoxy-quinazoline,
(1.64) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.67) 4-[(3-chloro-4-fluoro-phenyl)amino]-6-(tetrahydropyran-4-yloxy)-7-methoxy-quinazoline, and
(1.68) 4-[(3-chloro-4-fluoro-phenyl)amino]-6-(1-methylcarbonyl-piperidin-4-yloxy)-7-methoxy-quinazoline.

The pharmaceutical composition according to the invention may be administered in the form of a preparation suitable for inhalative, oral, intravenous, topical, subcutaneous, intramuscular, intraperitoneal, intranasal, transdermal or rectal administration.

In the pharmaceutical combinations according to the invention the active substances may be combined in a single preparation, e.g. as a fixed dose combination comprising the active ingredients in one formulation together, or contained in two or more separate formulations, e.g. as a kit of parts adapted for simultaneous, separate or sequential administration. Pharmaceutical compositions containing the active substances 1 and 2 in a single preparation are preferred according to the invention.

One embodiment of the invention is a pharmaceutical composition in the form of a preparation suitable for inhalation.

One embodiment of the invention is a pharmaceutical composition in the form of a preparation selected from among the inhalable powders, propellant-containing metered-dose aerosols and propellant-free inhalable solutions.

One embodiment of the invention is a pharmaceutical composition in the form of an inhalable powder which contains 1 and 2 in admixture with suitable physiologically acceptable excipients selected from among the monosaccharides, disaccharides, oligo- and polysaccharides, polyalcohols, salts, or mixtures of these excipients with one another.

One embodiment of the invention is a pharmaceutical composition in the form of an inhalable powder wherein the excipient has a maximum average particle size of up to 250 μm, preferably between 10 and 150 μm.

One embodiment of the invention is a pharmaceutical composition in the form of an inhalable powder which contains only the active substances 1 and 2 as its ingredients.

One embodiment of the invention is a pharmaceutical composition in the form of a propellant-containing inhalable aerosol which contains 1 and 2 in dissolved or dispersed form.

One embodiment of the invention is a pharmaceutical composition in the form of a propellant-containing inhalable aerosol that contains, as propellant gas, hydrocarbons such as n-propane, n-butane or isobutane or halohydrocarbons such as chlorinated and/or fluorinated derivatives of methane, ethane, propane, butane, cyclopropane or cyclobutane.

One embodiment of the invention is a pharmaceutical composition in the form of a propellant gas is TG11, TG12, TG134a, TG227 or mixtures thereof, preferably TG134a, TG227 or a mixture thereof.

One embodiment of the invention is a pharmaceutical composition in the form of a propellant-free inhalable solution which contains water, ethanol or a mixture of water and ethanol as solvent.

One embodiment of the invention is a pharmaceutical composition in the form of an inhalable solution wherein it optionally contains other co-solvents and/or excipients.

One embodiment of the invention is a pharmaceutical composition in the form of an is inhalable solution wherein it contains as co-solvents ingredients which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters.

One embodiment of the invention is a pharmaceutical composition in the form of an inhalable solution wherein it contains as excipients surfactants, stabilisers, complexing agents, antioxidants and/or preservatives, flavourings, pharmacologically acceptable salts and/or vitamins.

One embodiment of the invention is a method of treating an indication selected from indications (A):

prevention and treatment of diseases of the airways and lungs which are accompanied by increased or altered production of mucus and/or inflammatory and/or obstructive diseases of the airways such as
acute bronchitis, chronic bronchitis, chronic obstructive bronchitis (COPD),
cough, pulmonary emphysema,
allergic or non-allergic rhinitis or sinusitis, chronic sinusitis or rhinitis,
nasal polyposis, chronic rhinosinusitis, acute rhinosinusitis,
asthma, allergic bronchitis, alveolitis, Farmers disease, hyperreactive airways,
bronchitis or pneumonitis caused by infection, e.g. by bacteria or viruses or helminthes or fungi or protozoans or other pathogens,
pediatric asthma, bronchiectasis, pulmonary fibrosis,
adult respiratory distress syndrome, bronchial and pulmonary edema,
bronchitis or pneumonitis or interstitial pneumonitis caused by different origins, e.g. aspiration, inhalation of toxic gases, vapors,
bronchitis or pneumonitis or interstitial pneumonitis caused by heart failure, X-rays, radiation, chemotherapy,
bronchitis or pneumonitis or interstitial pneumonitis associated with collagenosis, e.g. lupus erythematodes, systemic scleroderma,
lung fibrosis, idiopathic pulmonary lung fibrosis (IPF), interstitial lung diseases or interstitial pneumonitis of different origin, including asbestosis, silicosis, M. Boeck or sarcoidosis, granulomatosis,
   cystic fibrosis or mucoviscidosis, or α1-antitrypsin deficiency,
   comprising administering a therapeutically effective amount of pharmaceutical composition according to the invention to a patient in need thereof.

One embodiment of the invention is a method wherein indication (A) is selected from chronic bronchitis, chronic obstructive bronchitis (COPD), chronic sinusitis, nasal polyposis, chronic rhinosinusitis, acute rhinosinusitis, and asthma.

One embodiment of the invention is a method of treating an indication selected from indications (B):

inflammatory or hypersecretory diseases of the gastrointestinal tract of various origins or polyps of the gastrointestinal tract of various origins such as
villous or adenomatous polyps of the large intestine, but also polyps in familial polyposis coli, in intestinal polyps in Gardner's syndrome, in polyps throughout the entire gastro-intestinal tract in Peutz-Jeghers Syndrome, in inflammatory pseudopolyps, in juvenile polyps, in colitis cystica profunda and in pneumatosis cystoides intestinales, acute or chronic inflammatory changes such as cholecystitis, Crohn's disease, ulcerative colitis, and ulcers or polyposis in the gastrointestinal tract or such as may occur in diseases of the gastrointestinal tract which are associated with increased secretions, such as Menetrier's disease, secreting adenomas and protein loss syndromes, or
diseases of the bile duct and gall bladder, e.g. gall stones or biliary concretion, inflammatory diseases of the joints, such as rheumatoid arthritis,
   or inflammatory diseases of the skin or the eyes,
   comprising administering a therapeutically effective amount of a pharmaceutical composition according to the invention to a patient in need thereof.

Preferred is a method according to the invention, wherein indication (B) is selected from Crohn's disease, ulcerative colitis or polyposis of the intestines.

One embodiment of the invention is the use of a pharmaceutical composition according to the invention for the manufacture of a medicament for treating an indication selected from indications (A):

prevention and treatment of diseases of the airways and lungs which are accompanied by increased or altered production of mucus and/or inflammatory and/or obstructive diseases of the airways such as
acute bronchitis, chronic bronchitis, chronic obstructive bronchitis (COPD), cough, pulmonary emphysema,
allergic or non-allergic rhinitis or sinusitis, chronic sinusitis or rhinitis,
nasal polyposis, chronic rhinosinusitis, acute rhinosinusitis,
asthma, allergic bronchitis, alveolitis, Farmers' disease, hyperreactive airways, bronchitis or pneumonits caused by infection, e.g. by bacteria or viruses or helminthes or fungi or protozoons or other pathogens,
pediatric asthma, bronchiectasis,
pulmonary fibrosis,
adult respiratory distress syndrome, bronchial and pulmonary edema,
bronchitis or pneumonitis or interstitial pneumonitis caused by different origins, e.g. aspiration, inhalation of toxic gases, vapors,
bronchitis or pneumonitis or interstitial pneumonitis caused by heart failure, X-rays, radiation, chemotherapy,
bronchitis or pneumonitis or interstitial pneumonitis associated with collagenosis, e.g. lupus erythematodes, systemic scleroderma,
lung fibrosis, idiopathic pulmonary lung fibrosis (IPF), interstitial lung diseases or interstitial pneumonitis of different origin, including asbestosis, silicosis, M. Boeck or sarcoidosis, granulomatosis,
   cystic fibrosis or mucoviscidosis, or α1-antitrypsin deficiency.

Preferred is the use according to the invention, wherein indication (A) is selected from chronic (obstructive) bronchitis (COPD), chronic sinusitis, nasal polyposis, chronic rhinosinusitis, acute rhinosinusitis, and asthma.

One embodiment of the invention is the use of a pharmaceutical composition according to the invention for the manufacture of a medicament for treating an indication selected from indications (B):

inflammatory or hypersecretory diseases of the gastrointestinal tract of various origins or polyps of the gastrointestinal tract of various origins such as
villous or adenomatous polyps of the large intestine, but also polyps in familial polyposis coil, in intestinal polyps in Gardner's syndrome, in polyps throughout the entire gastro-intestinal tract in Peutz-Jeghers Syndrome, in inflammatory pseudopolyps, in juvenile polyps, in colitis cystica profunda and in pneumatosis cystoides intestinales, acute or chronic inflammatory changes such as cholecystitis, Crohn's disease, ulcerative colitis, and ulcers or polyposis in the gastrointestinal tract or such as may occur in diseases of the gastrointestinal tract which are associated with increased secretions, such as Ménétrier's disease, secreting adenomas and protein loss syndromes, or diseases of the bile duct and gall bladder, e.g. gall stones or biliary concretion,
   inflammatory diseases of the joints, such as rheumatoid arthritis,
   or inflammatory diseases of the skin or the eyes,
   comprising administering a therapeutically effective amount of a pharmaceutical composition according to the invention to a patient in need thereof.

Preferably for the use according to the invention, wherein indication B is treated, the EGFR kinase inhibitor is selected from compounds 1.1 to 1.71.

Preferably for the use according to the invention, indication (B) is selected from Crohn's disease, ulcerative colitis or polyposis of the intestines.

The actives of the combinations according to the invention may be administered simultaneously, separately or sequentially. The preferred route of administration depends on the indication to be treated. In case of gastrointestinal indications, inflammatory joint, skin and eyes disorders both components 1 and 2 may be administered orally, intravenously or rectally, using suitable formulations known in the art, such as tablets, coated tablets, pills, granules or granular powder, syrups, emulsions, suspensions, solutions or suppositories, optionally together with inert and non-toxic pharmaceutically acceptable excipients or solvents. In case of inflammatory joint or skin disorders both components 1 and 2 also may be may be administered topically, using suitable formulations known in the art, such as ointments or transdermal patches. Furthermore, in case of inflammatory disorders of the eye both components 1 and 2 preferably are administered topically using suitable formulations such as ophthalmic solutions, eye drops or viscoelastic gels.

In case of respiratory indications and if administered separately or sequentially preferably at least one of components 1 and 2 is given by inhalative route. If component 1 is administered by inhalation component 2, administered separately, may be given for instance orally, intravenously, subcutaneously, by intramuscular injection, intraperitoneally, intranasally or transdermally, using suitable formulations known in the art, such as tablets, coated tablets, pills, granules or granular powder, aerosols, syrups, emulsions, suspensions, powders, solutions or transdermal patches, optionally together with inert and non-toxic pharmaceutically acceptable excipients or solvents. The same applies with respect to component 1, vice versa, if component 2 is administered by inhalation.

In case of respiratory indications both components 1 and 2 of the pharmaceutical combinations according to the invention preferably are administered by inhalation. Inhalable preparations according to the invention include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term carrier may optionally be used instead of the term excipient. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

Any aforementioned possible doses applicable for the combinations according to the invention are to be understood as referring to doses per single application. However, to these examples are not be understood as excluding the possibility of administering the combinations according to the invention multiple times. Depending on the medical need patients may receive also multiple inhalative applications. As an example patients may receive the combinations according to the invention for instance two or three times (e.g. two or three puffs with a powder inhaler, an MDI etc.) in the morning of each treatment day. As the aforementioned dose examples are only to be understood as dose examples per single application (i.e. per puff) multiple application of the combinations according to the invention leads to multiple doses of the aforementioned examples. The application of the compositions according to the invention can be for instance once a day, or depending on the duration of action of the agents twice a day, or once every 2 or 3 days.

Moreover it is emphazised that the aforementioned dosages are to be understood as examples of metered doses only. In other terms, the aforementioned doses are not to be understood as the effective doses of the combinations according to the invention that do in fact reach the lung. It is clear for the person of ordinary skill in the art that the delivered dose to the lung is generally lower than the metered dose of the administered active ingredients.

### A) Inhalable Powder Containing the Combinations of Active Substances 1 and 2 According to the Invention:

The inhalable powders according to the invention may contain 1 and 2 either on their own or in admixture with suitable physiologically acceptable excipients.

If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextran), polyalcohols (e.g. sorbitol, mannitol, xylitol), cyclodextrines (e.g. α-cyclodextrine, β-cyclodextrine, χ-cyclodextrine, methyl-β-cyclodextrine, hydroxypropyl-β-cyclodextrine), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain one or more physiologically acceptable excipients in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbuhaler® or using inhalers as disclosed for example in EP 237507A. Preferably, the inhalable powders according to the invention which contain physiologically acceptable excipient in addition to 1 and 2 are packed into capsules (to produce so-called inhalettes) which are used in inhalers as described, for example, in WO 94/28958.

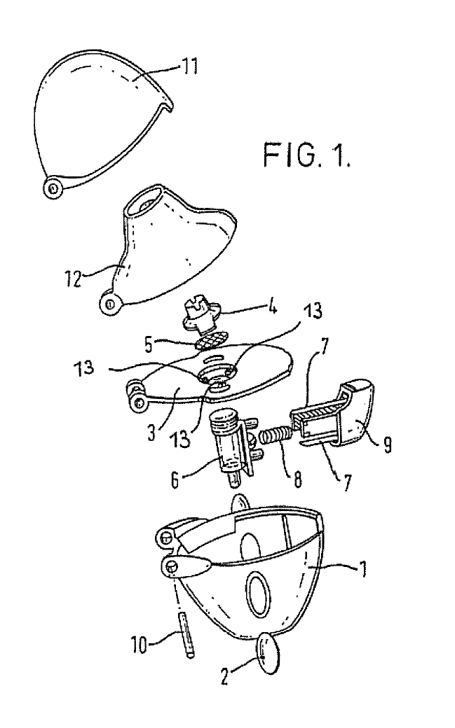

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured via a is screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as airholes 13 for adjusting the flow resistance.

If the inhalable powders according to the invention are packed into capsules (inhalers) for the preferred use described above, the quantities packed into each capsule should be 1 to 30 mg per capsule. These capsules contain, according to the invention, either together or separately, the doses of 1 and 2 or 2′ mentioned hereinbefore for each single dose.

### B) Propellant Gas-driven Inhalation Aerosols Containing the Combinations of Active Substances 1 and 2:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane) and TG227 (1,1,1,2,3,3,3-heptafluoropropane) and mixtures thereof, of which the propellant gases TG134a, to TG227 and mixtures thereof are preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to bpm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention. The present invention also relates to cartridges fitted with a suitable valve which can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

### C) Propellant-free Inhalable Solutions or Suspensions Containing the Combinations of Active Substances 1 and 2 According to the Invention:

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but preferably the maximum is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium editate, as stabiliser or complexing agent is unnecessary in the present formulation. Other embodiments may contain this compound or these compounds. In a preferred embodiment the content based on sodium editate is less than 100 mg/100 ml, preferably less than 50 mg/100 ml, more preferably less than 20 mg/100 ml. Generally, inhalable solutions in which the content of sodium editate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium editate. In another preferred embodiment, no sodium editate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 1004, preferably less than 50 μL, more preferably between 20 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

This nebuliser (Respimat®) can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in to WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active is substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO 94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is to thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well, are preferably made of plastics, e.g. by injection moulding. For medicinal purposes, physiologically safe materials are used.

FIGS. 6a/b of WO 97/12687, show the nebuliser (Respimat®) which can advantageously be used for inhaling the aqueous aerosol preparations according to the invention.

FIG. 6a of WO 97/12687 shows a longitudinal section through the atomiser with the spring biased while FIG. 6b of WO 97/12687 shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers or other stationary nebulisers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of active substances 1 and 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

According to the invention, inhalable solutions which contain the active substances 1 and 2 in a single preparation are preferred. The term “single preparation” also includes preparations which contain the two ingredients 1 and 2 in two-chamber cartridges, as disclosed for example in WO 00/23037. Reference is hereby made to this publication in its entirety.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated free-standing or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable to nebuliser which produces inhalable aerosols by means of ultrasound or compressed air by the Venturi principle or other methods.

1. Pharmaceutical composition comprising at least one EGFR kinase inhibitor 1 selected from the group consisting of compounds:
(1.1) 4-[(3-chloro-4-fluoro-phenyl)amino]-6-[2-(S)-6-methyl-2-oxo-morpholin-4-yl)-ethoxy]-7-methoxy-quinazoline,
(1.2) 4-[(3-chloro-4-fluoro-phenyl)amino]-6-[2-(2,2-dimethyl-6-oxo-morpholin-4-yl)-ethoxy]-7-methoxy-quinazoline,
(1.3) 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline,
(1.4) 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline,
(1.5) 4-[(3-chloro-4-fluoro-phenyl)amino]-6-[1-(tert-butyloxycarbonyl)-piperidin-4-yloxy]-7-methoxy-quinazoline,
(1.6) 4-[(3-chloro-4-fluoro-phenyl)amino]-6-(trans-4-amino-cyclohexan-1-yloxy)-7-methoxy-quinazoline,
(1.7) 4-[(3-chloro-4-fluoro-phenyl)amino]-6-(trans-4-methansulfonylamino-cyclohexan-1-yloxy)-7-methoxy-quinazoline,
(1.8) 4-[(3-chloro-4-fluoro-phenyl)amino]-6-(tetrahydropyran-3-yloxy)-7-methoxy-quinazoline,
(1.9) 4-[(3-chloro-4-fluoro-phenyl)amino]-6-(1-methyl-piperidin-4-yloxy)-7-methoxy-quinazoline,
(1.10) 4-[(3-chloro-4-fluoro-phenyl)amino]-6-{1-[(morpholin-4-yl)carbonyl]-piperidin-4-yloxy}-7-methoxy-quinazoline,
(1.11) 4-[(3-chloro-4-fluoro-phenyl)amino]-6-{1-[(methoxymethyl)carbonyl]-piperidin-4-yloxy}-7-methoxy-quinazoline,
(1.12) 4-[(3-chloro-4-fluoro-phenyl)amino]-6-(piperidin-3-yloxy)-7-methoxy-quinazoline,
(1.13) 4-[(3-chloro-4-fluoro-phenyl)amino]-6-[1-(2-acetylamino-ethyl)-piperidin-4-yloxy]-7-methoxy-quinazoline,
(1.14) 4-[(3-chloro-4-fluoro-phenyl)amino]-6-(tetrahydropyran-4-yloxy)-7-ethoxy-quinazoline
(1.15) 4-[(3-chloro-4-fluoro-phenyl)amino]-64(S)-tetrahydro furan-3-yloxy)-7-hydroxy-quinazoline,
(1.16) 4-[(3-chloro-4-fluoro-phenyl)amino]-6-(tetrahydropyran-4-yloxy)-7-(2-methoxy-ethoxy)-quinazoline,
(1.17) 4-[(3-chloro-4-fluoro-phenyl)amino]-6-{trans-4-[(dimethylamino)sulfonylamino]-cyclohexan-1-yloxy}-7-methoxy-quinazoline,
(1.18) 4-[(3-chloro-4-fluoro-phenyl)amino]-6-{trans-4-[(morpholin-4-yl)carbonylamino]-cyclohexan-1-yloxy}-7-methoxy-quinazoline,
(1.19) 4-[(3-chloro-4-fluoro-phenyl)amino]-6-{trans-4-[(morpholin-4-yl)sulfonylamino]-cyclohexan-1-yloxy}-7-methoxy-quinazoline,
(1.20) 4-[(3-chloro-4-fluoro-phenyl)amino]-6-(tetrahydropyran-4-yloxy)-7-(2-acetylamino-ethoxy)-quinazoline,
(1.21) 4-[(3-chloro-4-fluoro-phenyl)amino]-6-(tetrahydropyran-4-yloxy)-7-(2-methansulfonylamino-ethoxy)-quinazoline,
(1.22) 4-[(3-chloro-4-fluoro-phenyl)amino]-6-{1-[(piperidin-1-yl)carbonyl]-piperidin-4-yloxy}-7-methoxy-quinazoline,
(1.23) 4-[(3-chloro-4-fluoro-phenyl)amino]-6-(1-aminocarbonylmethyl-piperidin-4-yloxy)-7-methoxy-quinazoline,
(1.24) 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline,
(1.25) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.26) 4-[(3-chloro-4-fluoro-phenyl)amino]-6-(cis-4-{N-[(morpholin-4-yl)sulfonyl]-N-methyl-amino}-cyclohexan-1-yloxy)-7-methoxy-quinazoline,
(1.27) 4-[(3-chloro-4-fluoro-phenyl)amino]-6-(trans-4-ethansulfonylamino-cyclohexan-1-yloxy)-7-methoxy-quinazoline,
(1.28) 4-[(3-chloro-4-fluoro-phenyl)amino]-6-(1-methansulfonyl-piperidin-4-yloxy)-7-ethoxy-quinazoline,
(1.29) 4-[(3-chloro-4-fluoro-phenyl)amino]-6-(1-methansulfonyl-piperidin-4-yloxy)-7-(2-methoxy-ethoxy)-quinazoline,
(1.30) 4-[(3-chloro-4-fluoro-phenyl)amino]-641-(2-methoxy-acetyl)-piperidin-4-yloxy]-7-(2-methoxy-ethoxy)-quinazoline,
(1.31) 4-[(3-chloro-4-fluoro-phenyl)amino]-6-(cis-4-acetylamino-cyclohexan-1-yloxy)-7-methoxy-quinazoline,
(1.32) 4-[(3-ethinyl-phenyl)amino]-6-[1-(tert.-butyloxycarbonyl)-piperidin-4-yloxy]-7-methoxy-quinazoline,
(1.33) 4-[(3-ethinyl-phenyl)amino]-6-(tetrahydropyran-4-yloxy]-7-methoxy-quinazoline,
(1.34) 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline,
(1.35) 4-[(3-chloro-4-fluoro-phenyl)amino]-6-(cis-4-{N-[(4-methyl-piperazin-1-yl)-carbonyl]-N-methyl-amino}-cyclohexan-1-yloxy)-7-methoxy-quinazoline,
(1.36) 4-[(3-chloro-4-fluoro-phenyl)amino]-6-{cis-4-[(morpholin-4-yl)carbonylamino]-cyclohexan-1-yloxy}-7-methoxy-quinazoline,
(1.37) 4-[(3-chloro-4-fluoro-phenyl)amino]-6-{1-[2-(2-oxopyrrolidin-1-yl)ethyl]-piperidin-4-yloxy}-7-methoxy-quinazoline,
(1.38) 4-[(3-chloro-4-fluoro-phenyl)amino]-6-{1-[(morpholin-4-yl)carbonyl]-piperidin-4-yloxy}-7-(2-methoxy-ethoxy)-quinazoline,
(1.39) 4-[(3-ethinyl-phenyl)amino]-6-(1-acetyl-piperidin-4-yloxy)-7-methoxy-quinazoline,
(1.40) 4-[(3-ethinyl-phenyl)amino]-6-(1-methyl-piperidin-4-yloxy)-7-methoxy-quinazoline,
(1.41) 4-[(3-ethinyl-phenyl)amino]-6-(1-methansulfonyl-piperidin-4-yloxy)-7-methoxy-quinazoline,
(1.42) 4-[(3-chloro-4-fluoro-phenyl)amino]-6-(1-methyl-piperidin-4-yloxy)-7(2-methoxy-ethoxy)-quinazoline,
(1.43) 4-[(3-chloro-4-fluoro-phenyl)amino]-6-(1-isopropyloxycarbonyl-piperidin-4-yloxy)-7-methoxy-quinazoline,
(1.44) 4-[(3-chloro-4-fluoro-phenyl)amino]-6-(cis-4-methylamino-cyclohexan-1-yloxy)-7-methoxy-quinazoline,
(1.45) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.46) 4-[(3-ethinyl-phenyl)amino]-6-(piperidin-4-yloxy)-7-methoxy-quinazoline,
(1.47) 4-[(3-ethinyl-phenyl)amino]-6-[1-(2-methoxy-acetyl)-piperidin-4-yloxy]-7-methoxy-quinazoline,
(1.48) 4-[(3-ethinyl-phenyl)amino]-6-{1-[(morpholin-4-yl)carbonyl]-piperidin-4-yloxy}-7-methoxy-quinazoline,
(1.49) 4-[(3-chloro-4-fluoro-phenyl)amino]-6-{1-[(cis-2,6-dimethyl-morpholin-4-yl)carbonyl]-piperidin-4-yloxy}-7-methoxy-quinazoline,
(1.50) 4-[(3-chloro-4-fluoro-phenyl)amino]-6-{1-[(2-methyl-morpholin-4-yl)carbonyl]-piperidin-4-yloxy}-7-methoxy-quinazoline,
(1.51) 4-[(3-chloro-4-fluoro-phenyl)amino]-6-{1-[(S,S)-(2-oxa-5-aza-bicyclo [2.2.1]hept5-yl)carbonyl]-piperidin-4-yloxy}-7-methoxy-quinazoline,
(1.52) 4-[(3-chloro-4-fluoro-phenyl)amino]-6-{1-[(N-methyl-N-2-methoxyethyl-amino)-carbonyl]-piperidin-4-yloxy}-7-methoxy-quinazoline,
(1.53) 4-[(3-chloro-4-fluoro-phenyl)amino]-6-(1-ethyl-piperidin-4-yloxy)-7-methoxy-quinazoline,
(1.54) 4-[(3-chloro-4-fluoro-phenyl)amino]-6-{1-[(2-methoxyethyl)carbonyl]-piperidin-4-yloxy}-7-methoxy-quinazoline,
(1.55) 4-[(3-chloro-4-fluoro-phenyl)amino]-6-{1-[(3-methoxypropyl-amino)-carbonyl]-piperidin-4-yloxy}-7-methoxy-quinazoline,
(1.56) 4-[(3-chloro-4-fluoro-phenyl)amino]-6-[cis-4-(N-methansulfonyl-N-methyl-amino)-cyclohexan-1-yloxy]-7-methoxy-quinazoline,
(1.57) 4-[(3-chloro-4-fluoro-phenyl)amino]-6-[cis-4-(N-acetyl-N-methyl-amino)-cyclohexan-1-yloxy]-7-methoxy-quinazoline,
(1.58) 4-[(3-chloro-4-fluoro-phenyl)amino]-6-(trans-4-methylamino-cyclohexan-1-yloxy)-7-methoxy-quinazoline,
(1.59) 4-[(3-chloro-4-fluoro-phenyl)amino]-6-[trans-4-(N-methansulfonyl-N-methyl-amino)-cyclohexan-1-yloxy]-7-methoxy-quinazoline,
(1.60) 4-[(3-chloro-4-fluoro-phenyl)amino]-6-(trans-4-dimethylamino-cyclohexan-1-yloxy)-7-methoxy-quinazoline,
(1.61) 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline,
(1.62) 4-[(3-chloro-4-fluoro-phenyl)amino]-6-{1-[2-(2-oxo-3-methyl-imidazolidin-1-yl)-ethyl]-piperidin-4-yloxy}-7-methoxy-quinazoline,
(1.63) 4-[(3-chloro-4-fluoro-phenyl)amino]-6-{1-[2-(2-oxo-hexahydropyrimidin-1-yl)-ethyl]-piperidin-4-yloxy}-7-methoxy-quinazoline,
(1.64) 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline,
(1.65) 4-[(3-chloro-4-fluoro-phenyl)amino]-6-(1-methansulfonyl-piperidin-4-yloxy)-7-methoxy-quinazoline,
(1.66) 4-[(3-chloro-4-fluoro-phenyl)amino]-6-(1-cyano-piperidin-4-yloxy)-7-methoxy-quinazoline,
(1.67) 4-[(3-chloro-4-fluoro-phenyl)amino]-6-(tetrahydropyran-4-yloxy)-7-methoxy-quinazoline,
(1.68) 4-[(3-chloro-4-fluoro-phenyl)amino]-6-(1-methylcarbonyl-piperidin-4-yloxy)-7-methoxy-quinazoline,
(1.69) 4-[(3-chloro-4-fluoro-phenyl)amino]-6-(1-dimethylaminoacetyl-piperidin-4-yloxy)-7-methoxy-quinazoline,
(1.70) 4-[(3-chloro-4-fluoro-phenyl)amino]-6-{1-[(dimethylamino)carbonylmethyl]-piperidin-4-yloxy}-7-methoxy-quinazoline, and
(1.71) 4-[(3-chloro-4-fluoro-phenyl)amino]-6-(1-methansulfonyl-piperidin-4-yloxy)-quinazoline,
or a tautomer, racemate, enantiomer, diastereomer, or a pharmacologically acceptable acid addition salt thereof
and further comprising one or more additional active compounds 2 selected from the group consisting of beta-2 mimetics 2a, steroids 2b, PDE-IV inhibitors 2c, p38 MAP kinase inhibitors 2d, NK₁ antagonists 2e, anticholinergics 2f and endothelin antagonist 2g optionally together with one or more pharmaceutically acceptable excipients or carriers.

2. The pharmaceutical composition of claim 1 as a binary combination, containing an EGFR kinase inhibitor 1 and an active compound 2 selected from one of the classes 2a, 2b, 2c, 2d, 2e, 2f and 2g optionally together with one or more pharmaceutically acceptable excipients or carriers.

3. The pharmaceutical composition of claim 2, wherein the active compound 2 is a beta-2 mimetic 2a.

4. The pharmaceutical composition of claim 2, wherein the active compound 2 is a steroid 2b.

5. The pharmaceutical composition of claim 2, wherein the active compound 2 is a PDE-IV inhibitor 2c.

6. The pharmaceutical composition of claim 2, wherein the active compound 2 is a p38 MAP kinase inhibitor 2d.

7. The pharmaceutical composition of claim 2, wherein the active compound 2 is a NK₁ antagonists 2e.

8. The pharmaceutical composition of claim 2, wherein the active compound 2 is a anticholinergic 2f.

9. The pharmaceutical composition of claim 2, wherein the active compound 2 is a endothelin antagonist 2g.

10. The pharmaceutical composition of claim 1 as a ternary combination, containing an EGFR kinase inhibitor 1 and two active compounds selected from the class of beta-2 mimetics 2a and an active compound selected from the class of steroids 2b, optionally together with one or more pharmaceutically acceptable excipients or carriers.

11. The pharmaceutical composition of claim 1 as a ternary combination, containing two EGFR kinase inhibitors 1 and an active compound selected from one of the classes 2a, 2b, 2c, 2d, 2e, 2f and 2g, optionally together with one or more pharmaceutically acceptable excipients or carriers.

12. The pharmaceutical composition of claim 1 as a quarternary combination, containing two EGFR kinase inhibitors 1 and two active compounds selected from either one or from two different classes of 2a, 2b, 2c, 2d, 2e 2f and 2g optionally together with one or more pharmaceutically acceptable excipients or carriers.

13. The pharmaceutical composition of claim 1 as a quarternary combination, containing two EGFR kinase inhibitors 1 and two active compounds selected from either one or from two different classes of 2b, 2d, 2e, 2f and 2g, optionally together with one or more pharmaceutically acceptable excipients or carriers.

14-21. (canceled)

22. The pharmaceutical composition of claim 1, wherein the endothelin antagonist 2g is selected from the group consisting of ambrisentan 2g.1, sitaxsentan 2g.2 and TBC 3711 2g.3 and, a pharmacologically acceptable salt thereof.

23. The pharmaceutical composition of one of claim 1, wherein the EGFR kinase inhibitor is selected from the group consisting of:
(1.1) 4-[(3-chloro-4-fluoro-phenyl)amino]-6-[2-((S)-6-methyl-2-oxo-morpholin-4-yl)-ethoxy]-7-methoxy-quinazoline,
(1.2) 4-[(3-chloro-4-fluoro-phenyl)amino]-6-[2-(2,2-dimethyl-6-oxo-morpholin-4-yl)-ethoxy]-7-methoxy-quinazoline,
(1.8) 4-[(3-chloro-4-fluoro-phenyl)amino]-6-(tetrahydropyran-3-yloxy)-7-methoxy-quinazoline,
(1.12) 4-[(3-chloro-4-fluoro-phenyl)amino]-6-(piperidin-3-yloxy)-7-methoxy-quinazoline,
(1.13) 4-[(3-chloro-4-fluoro-phenyl)amino]-6-[1-(2-acetylamino-ethyl)-piperidin-4-yloxy]-7-methoxy-quinazoline,
(1.25) 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline,
(1.26) 4-[(3-chloro-4-fluoro-phenyl)amino]-6-(cis-4-{N-[(morpholin-4-yl)sulfonyl]-N-methyl-amino}-cyclohexan-1-yloxy)-7-methoxy-quinazoline,
(1.27) 4-[(3-chloro-4-fluoro-phenyl)amino]-6-(trans-4-ethansulfonylamino-cyclohexan-1-yloxy)-7-methoxy-quinazoline,
(1.28) 4-[(3-chloro-4-fluoro-phenyl)amino]-6-(1-methansulfonyl-piperidin-4-yloxy)-7-ethoxy-quinazoline,
(1.33) 4-[(3-ethinyl-phenyl)amino]-6-(tetrahydropyran-4-yloxy]-7-methoxy-quinazoline,
(1.45) 4-[(3-chloro-4-fluoro-phenyl)amino]-6-{cis-4-[N-(2-methoxy-acetyl)-N-methyl-amino]-cyclohexan-1-yloxy}-7-methoxy-quinazoline,
(1.46) 4-[(3-ethinyl-phenyl)amino]-6-(piperidin-4-yloxy)-7-methoxy-quinazoline,
(1.47) 4-[(3-ethinyl-phenyl)amino]-6-[1-(2-methoxy-acetyl)-piperidin-4-yloxy]-7-methoxy-quinazoline,
(1.48) 4-[(3-ethinyl-phenyl)amino]-6-{1-[(morpholin-4-yl)carbonyl]-piperidin-4-yloxy}-7-methoxy-quinazoline,
(1.62) 4-[(3-chloro-4-fluoro-phenyl)amino]-6-{1-[2-(2-oxo-3-methyl-imidazolidin-1-yl)-ethyl]-piperidin-4-yloxy}-7-methoxy-quinazoline,
(1.64) 4-[(3-chloro-4-fluoro-phenyl)amino]-6-[2-(2,2-dimethyl-6-oxo-morpholin-4-yl)-ethoxy]-7-[(S)-(tetrahydro furan-2-yl)methoxy]-quinazoline,
(1.67) 4-[(3-chloro-4-fluoro-phenyl)amino]-6-(tetrahydropyran-4-yloxy)-7-methoxy-quinazoline,
(1.68) 4-[(3-chloro-4-fluoro-phenyl)amino]-6-(1-methylcarbonyl-piperidin-4-yloxy)-7-methoxy-quinazoline,
(1.69) 4-[(3-chloro-4-fluoro-phenyl)amino]-6-(1-dimethylaminoacetyl-piperidin-4-yloxy)-7-methoxy-quinazoline,
(1.70) 4-[(3-chloro-4-fluoro-phenyl)amino]-6-{1-[(dimethylamino)carbonylmethyl]-piperidin-4-yloxy}-7-methoxy-quinazoline and
(1.71) 4-[(3-chloro-4-fluoro-phenyl)amino]-6-(1-methansulfonyl-piperidin-4-yloxy)-quinazoline,
or a pharmaceutically acceptable salt thereof.

24. Pharmaceutical composition according to claim 1, characterised in that it is in the form of a preparation suitable for inhalative, oral, intravenous, topical, subcutaneous, intramuscular, intraperitoneal, intranasal, transdermal or rectal administration.

25-36. (canceled)

37. A method of treating an indication selected the group consisting of prevention and treatment of diseases of the airways and lungs which are accompanied by increased or altered production of mucus and/or inflammatory and/or obstructive diseases of the airways
comprising administering a therapeutically effective amount of pharmaceutical composition according to any of claim 1 to a patient in need thereof.

38. (canceled)

39. A method of treating an indication selected from the group consisting of inflammatory or hypersecretory diseases of the gastrointestinal tract of various origins or polyps of the gastrointestinal tract of various origins
comprising administering a therapeutically effective amount of a pharmaceutical composition according to claim 1 to a patient in need thereof.

40-45. (canceled)

46. The method of claim 37 wherein the indication is selected from:
acute bronchitis,
chronic bronchitis,
chronic obstructive bronchitis (COPD),
cough,
pulmonary emphysema,
allergic or non-allergic rhinitis,
sinusitis,
chronic sinusitis
rhinitis,
nasal polyposis,
chronic rhinosinusitis,
acute rhinosinusitis,
asthma,
allergic bronchitis,
alveolitis,
Farmers'disease,
hyperreactive airways,
bronchitis or pneumonits caused by infection,
pediatric asthma,
bronchiectasis,
pulmonary fibrosis,
adult respiratory distress syndrome,
bronchial and pulmonary edema,
bronchitis, pneumonitis or interstitial pneumonitis caused by aspiration, inhalation of toxic gases, vapors,
bronchitis, pneumonitis or interstitial pneumonitis caused by heart failure, X-rays, radiation, chemotherapy,
bronchitis, pneumonitis or interstitial pneumonitis associated with collagenosis,
lung fibrosis,
idiopathic pulmonary lung fibrosis (IPF),
interstitial lung diseases or interstitial pneumonitis caused by asbestosis, silicosis, M. Boeck or sarcoidosis, granulomatosis,
cystic fibrosis, mucoviscidosis, and
α1 antitrypsin deficiency.

47. The method of claim 46, wherein the indication is selected from chronic bronchitis, chronic obstructive bronchitis (COPD), chronic sinusitis, nasal polyposis, chronic rhinosinusitis, acute rhinosinusitis, and asthma.

48. the method of claim 39, where the indication is selected from:
villous or adenomatous polyps of the large intestine,
polyps in familial polyposis coli,
polyps in intestinal polyps in Gardner's syndrome,
polyps throughout the entire gastro-intestinal tract in Peutz-Jeghers Syndrome,
inflammatory pseudopolyps,
juvenile polyps,
colitis cystica profunda and in pneumatosis cystoides intestinales,
acute or chronic inflammatory changes such as cholecystitis, Crohn's disease, ulcerative colitis, and ulcers,
polyposis in the gastrointestinal associated with increased secretions caused by Ménétrier's disease, secreting adenomas and protein loss syndromes,
diseases of the bile duct and gall bladder,
inflammatory diseases of the joints, and
inflammatory diseases of the skin or the eyes.

49. The method of claim 48, wherein the indication is selected from Crohn's disease, ulcerative colitis and polyposis of the intestines.
